SEQUENCE LISTING (143 sequences)

SEQ ID NO: 1-9 - skipped (9 entries)

SEQ ID NO: 10 - protein, 132 aa, Bacteriophage Q-beta
AKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 11 - protein, 328 aa, Bacteriophage Q-beta
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAYWLLIAGGGSGSKPDPVIPDPPIDPPPGTGKYTCPFAIWSLEEVYEPPTKNRPWPIYNAVELQPREFDVALKDLLGNTKWRDWDSRLSYTTFRGCRGNGYIDLDATYLATDQAMRDQKYDIREGKKPGAFGNIERFIYLKSINAYCSLSDIAAYHADGVIVGFWRDPSSGGAIPFDFTKFDKTKCPIQAVIVVPRA

SEQ ID NO: 12 - protein, 129 aa, Bacteriophage R17
ASNFTQFVLVNDGGTGNVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 13 - protein, 130 aa, Bacteriophage fr
MASNFEEFVLVDNGGTGDVKVAPSNFANGVAEWISSNSRSQAYKVTCSVRQSSANNRKYTVKVEVPKVATQVQGGVELPVAAWRSYMNMELTIPVFATNDDCALIVKALQGTFKTGNPIATAIAANSGIY

SEQ ID NO: 14 - protein, 130 aa, Bacteriophage GA
MATLRSFVLVDNGGTGNVTVVPVSNANGVAEWLSNNSRSQAYRVTASYRASGADKRKYAIKLEVPKIVTQVVNGVELPGSAWKAYASIDLTIPIFAATDDVTVISKSLAGLFKVGNPIAEAISSQSGFYA

SEQ ID NO: 15 - protein, 132 aa, Bacteriophage SP
MAKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAY

SEQ ID NO: 16 - protein, 329 aa, Bacteriophage SP
AKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAYWAALLVASSGGGDNPSDPDVPVVPDVKPPDGTGRYKCPFACYRLGSIYEVGKEGSPDIYERGDEVSVTFDYALEDFLGNTNWRNWDQRLSDYDIANRRRCRGNGYIDLDATAMQSDDFVLSGRYGVRKVKFPGAFGSIKYLLNIQGDAWLDLSEVTAYRSYGMVIGFWTDSKSPQLPTDFTQFNSANCPVQTVIIIPS

SEQ ID NO: 17 - protein, 130 aa, Bacteriophage MS2
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 18 - protein, 133 aa, Bacteriophage M11
MAKLQAITLSGIGKKGDVTLDLNPRGVNPTNGVAALSEAGAVPALEKRVTISVSQPSRNRKNYKVQVKIQNPTSCTASGTCDPSVTRSAYSDVTFSFTQYSTVEERALVRTELQALLADPMLVNAIDNLNPAY

SEQ ID NO: 19 - protein, 133 aa, Bacteriophage MX1
MAKLQAITLSGIGKNGDVTLNLNPRGVNPTNGVAALSEAGAVPALEKRVTISVSQPSRNRKNYKVQVKIQNPTSCTASGTCDPSVTRSAYADVTFSFTQYSTDEERALVRTELKALLADPMLIDAIDNLNPAY

SEQ ID NO: 20 - protein, 330 aa, Bacteriophage NL95
MAKLNKVTLTGIGKAGNQTLTLTPRGVNPTNGVASLSEAGAVPALEKRVTVSVAQPSRNRKNYKVQIKLQNPTACTKDACDPSVTRSGSRDVTLSFTSYSTERERALIRTELAALLKDDLIVDAIDNLNPAYWAALLAASPGGGNNPYPGVPDSPNVKPPGGTGTYRCPFACYRRGELITEAKDGACALYACGSEALVEFEYALEDFLGNEFWRNWDGRLSKYDIETHRRCRGNGYVDLDASVMQSDEYVLSGAYDVVKMQPPGTFDSPRYYLHLMDGIYVDLAEVTAYRSYGMVIGFWTDSKSPQLPTDFTRFNRHNCPVQTVIVIPSL

SEQ ID NO: 21 - protein, 129 aa, Bacteriophage f2
ASNFTQFVLVNDGGTGNVTVAPSNFANGVAEWISSNSRSQAYKVTCSVRQSSAQNRKYTIKVEVPKVATQTVGGVELPVAAWRSYLNLELTIPIFATNSDCELIVKAMQGLLKDGNPIPSAIAANSGIY

SEQ ID NO: 22 - protein, 128 aa, Bacteriophage PP7
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 23 - protein, 132 aa, Bacteriophage Q-beta
AKLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 24 - protein, 132 aa, Bacteriophage Q-beta
AKLETVTLGKIGKDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 25 - protein, 132 aa, Bacteriophage Q-beta
ARLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 26 - protein, 132 aa, Bacteriophage Q-beta
AKLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 27 - protein, 132 aa, Bacteriophage Q-beta
ARLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 28 - protein, 184 aa, Hepatitis B virus
MDIDPYEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 29 - protein, 183 aa, Hepatitis B virus
MDIDPYKEFGATVELLSFLPSDFFPSVRDLLDTASALYREALESPEHCSPHHTALRQAILCWGELMTLATWVGGNLEDPISRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRGSQC

SEQ ID NO: 30 - protein, 183 aa, Hepatitis B virus
MDIDPYKEFGATVELLSFLPSDFFPSVRDLLDTASALYREALESPEHCSPHHTALRQAILCWGELMTLATWVGGNLEDPTSRDLVVSYVNTNMGLKFRQLLWFHISCLTFGRETVIEYLVSFGVWIRTPPAYRPTNAPILSTLPETCVIRRRGRSPRRRTPSPRRRRSQSPRRRRSQSRGSQC

SEQ ID NO: 31 - protein, 212 aa, Hepatitis B virus
MQLFHLCLIISCSCPTVQASKLCLGWLWGMDIDPYKEFGATVELLSFLPSDFFPSVRDLLDTASALYREALESPEHCSPHHTALRQAILCWGELMTLATWVGGNLEDPISRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 32 - protein, 212 aa, Hepatitis B virus
MQLFHLCLIISCSCPTVQASKLCLGWLWGMDIDPYKEFGATVELLSFLPSDFFPSVRDLLDNASALYREALESPEHCSPHHTALRQAILCWGELMTLATWVGGNLEDPISRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 33 - protein, 183 aa, Hepatitis B virus
MDIDPYKEFGATVELLSFLPTDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETCVVRRRGRSPRRRTPSPRRRRSQSPRRRRSQSRESQC

SEQ ID NO: 34 - protein, 212 aa, Hepatitis B virus
MQLFHLCLIISCSCPTVQASKLCLGWLWGMDIDPYKEFGATVELLSFLPSDFFPSVRDLLDTASALYREALESPEHCSPHHTALRQAILCWGDLMTLATWVGGNLEDPVSRDLVVSYVNTNVGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 35 - protein, 212 aa, Hepatitis B virus
MQLFHLCLIISCSCPTVQASKLCLGWLWDMDIDPYKEFGATVELLSFLPSDFFPSVRDLLDTASALYREALESPEHCSPHHTALRQAILCWGDLMTLATWVGGNLEDPVSRDLVVSYVNTNVGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 36 - protein, 212 aa, Hepatitis B virus
MQLFHLCLIISCSCPTVQASKLCLGWLWGMDIDPYKEFGATVELLSFLPSDFFPSVRDLLDTASALYREALESPEHCSPQHTALRQAILCWGELMTLATWVGGNLEDPISRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 37 - protein, 212 aa, Hepatitis B virus
MQLFHLCLIISCSCPTVQASKLCLGWLWGMDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYKPPNAPILSTLPETTVVRRRGRSPRRRTPSPRRRRSQSPRRRRSQSRGSQC

SEQ ID NO: 38 - protein, 183 aa, Hepatitis B virus
MDIDPYKEFGATVELLSFLPSDFFPSVRDLLDTASALFRDALESPEHCSPHHTALRQAILCWGELMTLATWVGGNLEDPASRDLVVSYVNTNMGLKFRQLLWFHISCLTFGRDTVIEYLVSFGVWIRTPPAYRPSNAPILSTLPETCVVRRRGRSPRRRTPSPRRRRSQSPRRRRSQSRESQC

SEQ ID NO: 39 - protein, 183 aa, Hepatitis B virus
MDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 40 - protein, 212 aa, Hepatitis B virus
MQLFHLCLIISCSCPTVQASKLCLGWLWGMDIDPYKEFGATVELLSFLPSDFFPSVRDLLDTASALYREALESPEHCSPHHTALRHAILCWGDLRTLATWVGGNLEDPISRDLVVSYVNTNMGLKFRQLLYFHISCLTFGRETVIEYLVSFGVWIRTPPAYRPPNAPILSTLPETTVVRRRGRSPRRRTPSPRRRRSQSPRRRRSQSRESQC

SEQ ID NO: 41 - protein, 212 aa, Hepatitis B virus
MQLFHLCLIISCSCPTVQASKLCLGWLWDMDIDPYKEFGATVELLSFLPSDFFPSVRDLLDTASALFRDALESPEHCSPHHTALRQAILCWGELMTLATWVGANLEDPASRDLVVSYVNTNMGLKFRQLLWFHISCLTFGRETVIEYLVSFGVWIRTPQAYRPPNAPILSTLPETCVVRRRGRSPRRRTPSPRRRRSQSPRRRRSQSRESQC

SEQ ID NO: 42 - protein, 183 aa, Artificial Sequence
  Synthetic human Hepatitis B virus core protein gene
MDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVLEYLVSFGVWIRTPPAYRPPNAPILSTLPETTVVRRRGRSPRRRTPSPRRRRSQSPRRRRSQSRESQC

SEQ ID NO: 43 - protein, 212 aa, Hepatitis B virus
MQLFHLCLIISCSCPTVQASKLCLGWLWGMDIDPYKEFGATVELLSFLPSDFFPSVRDLLDTASALYREALESPEHCSPHHTALRQAILCWGDLMSLATWVGVNLEDPISRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 44 - protein, 183 aa, Hepatitis B virus
MDIDPYKEFGATVELLSFLPSDFFPSVRDLLDTASALYRDALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 45 - protein, 183 aa, Hepatitis B virus
MDIDPYKEFGATVELLSFLPSDFFPSVRDLLDTASALYREALESPEHCSPHHTALRQAILCWGDLMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 46 - protein, 183 aa, Hepatitis B virus
MDIDPYKEFGATVELLSFLPSDFFPSVRDLLDTASALYRDALESPEHCSPHHTALRQAILCWGELMTLATWVGANLEDPASRDLVVSYVNTNMGLKFRQLLWFHISCLTFGRETVIEYLVSFGVWIRTPPAYRPPNAPILSTLPETTVVRRRGRTPRRRTPSPRRRRSQSPRRRRSQSRESQC

SEQ ID NO: 47 - protein, 212 aa, Hepatitis B virus
MQLFHLCLIISCSCPTVQASKLCLGWLWGMDIDPYKEFGATVELLSFLPSDFFPSVRDLLDTASALYRDALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 48 - protein, 212 aa, Hepatitis B virus
MQLFHLCLIISCSCPTVQASKLCLGWLWGMDIDPYKEFGATVELLSFLPSDFFPSVRDLLDTASALYREALESPEHCSPHHTALRQAILCWGDLMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 49 - protein, 212 aa, Hepatitis B virus
MQLFHLCLIISCTCPTVQASKLCLGWLWGMDIDPYKQFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVAFGVWIRTPPAYRPPNAPILSTLPETTVVRRRGRSPRRRTPSPRRRRSQSPRRRRSQSRESQC

SEQ ID NO: 50 - protein, 212 aa, Hepatitis B virus
MQLFHLCLIISCSCPTVQASKLCLGWLWGMDIDPYKEFGATVELLSFLPSDFFPSVRDLLDTASALYREAFECSEHCSPHHTALRQAILCWGELMTLATWVGGNLEDPISRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 51 - protein, 212 aa, Hepatitis B virus
  MISC_FEATURE (28)..(28): Xaa can be any amino acid
MQLFHLCLIISCSCPTVQASKLCLGWLXDMDIDPYKEFGATVELLSFLPSDFFPSVRDLLDTASALYREALESPEHCSPHHTALRQAILCWGDLITLSTWVGGNLEDPTSRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRTQSRESQC

SEQ ID NO: 52 - protein, 212 aa, Hepatitis B virus
MQLFHLCLIISCSCPTVQASKLCLGWLWGMDIDPYKEFGATVELLSFLPSDFFPSVRDLLDNASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 53 - protein, 212 aa, Hepatitis B virus
MQLFHLCLIISCSCPTVQASKLCLGWLWGMDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHICCLTFGRETVIEYLVSFGVWIRTPPAYRPPNAPILSTLPETTVVRRRGRSPRRRTPSPRRRRSQSPRRRRSQSRESQC

SEQ ID NO: 54 - protein, 212 aa, Hepatitis B virus
MQLFHLCLIISCSCPTVQASKLCLGWLWGMDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSREPQC

SEQ ID NO: 55 - protein, 212 aa, Hepatitis B virus
MQLFHLCLIISCSCPTVQASKLCLGWLWGMDIDPYKEFGATVELLSFLPSDFFPSVRDLLSTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 56 - protein, 212 aa, Hepatitis B virus
MQLFHLCLIISCSCPTVQASKLCLGWLWGMDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILLTLPETTVVRRRGRSPRRRTPSPRRRRSQSPRRRRSQSRESQC

SEQ ID NO: 57 - protein, 212 aa, Hepatitis B virus
MQLFHLCLIISCSCPTVQASKLCLGWLWGMDIDPYKEFGATVELLSFLPSDFFPSVRDLLDTASALYREALESPEHCSPHHTALRQAILCWGDLMTLATWVGVNLEDPASRDLVVSYVNTNMGLKFKQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 58 - protein, 212 aa, Hepatitis B virus
MQLFHLCLIISCSCPTVQASKLCLGWLWGMDIDPYKEFGATVELLSFLPSDFFPSVRDLLDTAAALYRDALESPEHCSPHHTALRQAILCWGELMTLATWVGTNLEDPASRDLVVSYVNTNMGLKFRQLLWFHISCLTFGRETVLEYLVSFGVWIRTPPAYRPPNAPILSTLPETTVVRRRGRSPRRRTPSPRRRRSQSPRRRRSQSRESQC

SEQ ID NO: 59 - protein, 183 aa, Hepatitis B virus
MDIDPYKEFGASMELLSFLPSDFYPSVRDLLDTASALYREALESPEHCTPHHTALRQAILCWGELMTLATWVGGNLQDPTSRDLVVSYVNTNMGLKFRQLLWFHVSCLTFGRETVVEYLVSFGVWIRTPQAYRPPNAPILSTLPETCVVRRRGRSPRRRTPSPRRRRSQSPRRRRSQSRESQC

SEQ ID NO: 60 - protein, 183 aa, Hepatitis B virus
MDIDPYKEFGATVELLSFLPSDFFPSVRDLLDTASALYREALESPEHCSPHHTALRHVFLCWGDLMTLATWVGGNLEDPTSRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 61 - protein, 212 aa, Hepatitis B virus
MQLFHLCLIISCSCPTVQASKLCLGWLWGMDIDPYKEFGATVELLSFLPSDFFPSVRDLLDTASALYREALESPEHCSPHHTALRQAILCWGDLTTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 62 - protein, 212 aa, Hepatitis B virus
MQLFHLCLIISCSCPTVQASKLCLGWLWGMDIDPYKEFGATVELLSFLPSDFFPSVRDLLDTASALYRDALESPEHCSPHHTALRQAILCWGELMTLATWVGVNLEDPASRDLVVSYVNTNMGLKFRQLLWFHISCLIFGRETVIEYLVSFGVWIRTPPAYRPPNAPILSTLPETTVVRRRGRSPRRRTPSPRRRRSQSPRRRRSQSRESQC

SEQ ID NO: 63 - protein, 183 aa, Hepatitis B virus
MDIDPYKEFGATVELLSFLPSDFFPSVRDLLDTASALYREALESPEHCSPHHTALRQAILCWGDLMTLATWVGVNLEDPVSRDLVVSYVNTNVGLKFRQLLWFHISCLTFGRETVIEYLVSFGVWIRTPPAYRPPNAPILSTLPETTVVRRRGRSPRRRTPSPARRRSQSPRRRRSQSRESQC

SEQ ID NO: 64 - protein, 213 aa, Hepatitis B virus
MQLFHLCLIISCSCPTVQASKLCLGWLWGMDIDPYKEFGATVELLSFLPSDFFPSVRDLLDTASALYREALESPEHCSPHHTALRQAILCWGDLMNLATWVGGNLEDPVSRDLVVGYVNTTVGLKFRQLLWFHISCLTFGRETVIEYLVSFGVWIRTPPAYRPPNAPILSTLPETTVVRRRGRSPRRRTPSPPRRRRSQSPRRRRSQSRESQC

SEQ ID NO: 65 - protein, 183 aa, Hepatitis B virus
MDIDPYKEFGATVELLSFLPSDFFPSVRDLLDTASALYRDALESPEHCSPHHTALRQAILCWGDLMTLATWVGVNLEDPASRDLVVSYVNTNMGLKFRQLLWFHISCLTFGRETVIEYLVSFGVWIRTPPAYRPPNAPILSTLPETTVVRRRGRTPRRRTPSPRRRRSQSPRRRRSQSRESQC

SEQ ID NO: 66 - protein, 212 aa, Hepatitis B virus
MQLFHLCLIISCSCPTVQASKLCLGWLWGMDIDPYKEFGATVELLSFLPSDFFPSVRALLDTASALYREALESPEHCSPHHTALRQAILCWGELMTLATWVGVNLEDPASRDLVVSYVNTNMGLKFRQILWFHISCLTFGRETVIEYLVSFGVWIRTPPAYRPPNAPILSTLPETTVVRRRGRSPRRRTPSPRRRRSQSPRRRRSQSRESQC

SEQ ID NO: 67 - protein, 212 aa, Hepatitis B virus
MQLFHLCLIISCSCPTVQASKLCLGWLWGMDIDPYKEFGATVELLSFLPSDFFPSVRDLLDTASALYREALESPEHCSPHHTALRQAILCWGDLMTLATWVGVNLEDPATRDLVVSYVNTNVGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 68 - protein, 212 aa, Hepatitis B virus
MQLFHLCLIISCSCPTVQASKLCLGWLWGMDIDPYKEFGATVELLSFLPSDFFPSVRDLLDTASALYREALESPEHCSPHHTALRQRILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRTRSQSRESQC

SEQ ID NO: 69 - protein, 212 aa, Hepatitis B virus
MQLFHLCLVISCSCPTVQASKLCLGWLWGMDIDPYKEFGATVELLSFLPSDFFPSVRDLLDTAAALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRGRSPRRRTPSPRRRRSQSPRRRRSQSRESQC

SEQ ID NO: 70 - protein, 212 aa, Hepatitis B virus
MQLFHLCLIISCSCPTVQASKLCLGWLWGMDIDPYKEFGATVELLSFLPSAFFPSVRDLLDTASALYREALESPEHCSPHHTALRQAILCWGDLMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 71 - protein, 183 aa, Hepatitis B virus
MDIDPYKEFGATVELLSFLPSDFFPSVRDLLDTAAALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRGRSPRRRTPSPRRRRSQSPRRRRSQSRESQC

SEQ ID NO: 72 - protein, 183 aa, Hepatitis B virus
MDIDPYKEFGATVELLSFLPSDFFPSVRDLLDTASALYREALESPEHCSPHHTALRQAILCWGELMTLATWVGGNLEDPISRDLVVSYVNTNMGLKFRQLLWFHISCLTFGRETVIEYLVSFGVWIRTPPAYRPPNAPILSTLPETCVVRRRGRSPRRRTPSPRRRRSQSPRRRRSQSRGSQC

SEQ ID NO: 73 - protein, 188 aa, Hepatitis B virus
MDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSTNC

SEQ ID NO: 74 - protein, 217 aa, Hepatitis B virus
MYLFHLCLVFACVPCPTVQASKLCLGWLWDMDIDPYKEFGSSYQLLNFLPLDFFPDLNALVDTAAALYEEELTGREHCSPHHTAIRQALVCWEELTRLITWMSENTTEEVRRIIVDHVNNTWGLKVRQTLWFHLSCLTFGQHTVQEFLVSFGVWIRTPAPYRPPNAPILSTLPEHTVIRRRGGSRAARSPRRRTPSPRRRRSQSPRRRRSQSPASNC

SEQ ID NO: 75 - protein, 262 aa, Hepatitis B virus
MDVNASRALANVYDLPDDFFPKIEDLVRDAKDALEPYWKSDSIKKHVLIATHFVDLIEDFWQTTQGMHEIAEAIRAVIPPTTAPVPSGYLIQHDEAEEIPLGDLFKEQEERIVSFQPDYPITARIHAHLKAYAKINEESLDRARRLLWWHYNCLLWGEATVTNYISRLRTWLSTPEKYRGRDAPTIEAITRPIQVAQGGRKTSTATRKPRGLEPRRRKVKTTVVYGRRRSKSRERRASSPQRAGSPLPRSSSSHHRSPSPRK

SEQ ID NO: 76 - protein, 305 aa, Hepatitis B virus
MWDLRLHPSPFGAACQGIFTSSLLLFLVTVPLVCTIVYDSCLCMDINASRALANVYDLPDDFFPKIDDLVRDAKDALEPYWRNDSIKKHVLIATHFVDLIEDFWQTTQGMHEIAEALRAIIPATTAPVPQGFLVQHEEAEEIPLGELFRYQEERLTNFQPDYPVTARIHAHLKAYAKINEESLDRARRLLWWHYNCLLWGEPNVTNYISRLRTWLSTPEKYRGKDAPTIEAITRPIQVAQGGRNKTQGVRKSRGLEPRRRRVKTTIVYGRRRSKSRERRAPTPQRAGSPLPRTSRDHHRSPSPRE

SEQ ID NO: 77 - protein, 185 aa, Hepatitis B virus
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 78 - protein, 152 aa, Hepatitis B virus
MDIDPYKEFGATVELLSFLPSDFFPSVRDLLDTAAALYRDALESPEHCSPHHTALRQAILCWGDLMTLATWVGTNLEDGGKGGSRDLVVSYVNTNVGLKFRQLLWFHISCLTFGRETVLEYLVSFGVWIRTPPAYRPPNAPILSTLPETTVV

SEQ ID NO: 79 - DNA, 3635 bp, Artificial Sequence
  plasmid pAP283-58
cgagctcgcccctggcttatcgaaattaatacgactcactatagggagaccggaattcgagctcgcccggggatcctctagaattttctgcgcacccatcccgggtggcgcccaaagtgaggaaaatcacatggcaaataagccaatgcaaccgatcacatctacagcaaataaaattgtgtggtcggatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgcttaagcttgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgttccgcaccccggaaatggtcaccgaaccaatcagcagggtcatcgctagccagatcctctacgccggacgcatcgtggccggcatcaccggcgcacacagtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgcttcaacggcctcaacctactactgggctgcttcctaatgcaggagtcgcataagggagagcgtcgatatggtgcactctcagtacaatctgctctgatgccgcatagttaagccaactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgcgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgacgagcgcagcgagtcagtgagcgaggaagcggaagagcgcccaatacgcaaaccgcctctccccgcgcgttggccgattcattaatgcagctgtggtgtcatggtcggtgatcgccagggtgccgacgcgcatctcgactgcatggtgcaccaatgcttctggcgtcaggcagccatcggaagctgtggtatggccgtgcaggtcgtaaatcactgcataattcgtgtcgctcaaggcgcactcccgttctggataatgttttttgcgccgacatcataacggttctggcaaatattctgaaatgagctgttgacaattaatcatcgaactagttaactagtacgcaagttcacgtaaaaagggtatcgcggaatt

SEQ ID NO: 80 - protein, 131 aa, Artificial Sequence
  AP205 coat protein
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 81 - protein, 131 aa, Artificial Sequence
  AP205 coat protein
MANKTMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 82 - DNA, 3607 bp, Artificial Sequence
  plasmid pAP281-32
cgagctcgcccctggcttatcgaaattaatacgactcactatagggagaccggaattcgagctcgcccggggatcctctagattaacccaacgcgtaggagtcaggccatggcaaataagacaatgcaaccgatcacatctacagcaaataaaattgtgtggtcggatccaactcgtttatcaactacattttcagcaagtctgttacgccaacgtgttaaagttggtatagccgaactgaataatgtttcaggtcaatatgtatctgtttataagcgtcctgcacctaaaccgaaggtcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgcttaagcttgtattctatagtgtcacctaaatcgtatgtgtatgatacataaggttatgtattaatggtagccgcgttctaacgacaatatgtacaagcctaattgtgtagcatctggcttactgaagcagaccctatcatctctctcgtaaactgccgtcagagtcggttgggttggacagacctctgagtttctggtaacgccgttccgcaccccggaaatggtcaccgaaccattcagcagggtcatcgctagccagatcctctacgccggacgcatcgtggcccgcatcaccggcgccacaggtgcggtgctggcgcctatatcgccgacatcaccgatggggaagatcgggctcgccacttcgggctcatgatcgctggtttccgcctgggtatggtggcaggccccgtggcccgggggactgttgggcgccatctccttgcatgcaccattccttgcggcggcggtgctcaacggcctcaacctactactgggctgcttcctaatgcaggagtcgcataagggagagcgtcgatatggtgcactctcagtacaatctgctctgatgccgcatagttaagccaactccgctatcgctacgtgactgggtcatggctgcgccccgacacccgccaacacccgctgacgcgccctgacgggcttgtctgcttccggcatccgcttacagacaagctgtgaccgtctccgggagctgcatgtgtcagaggttttcaccgtcatcaccgaaacgcgcgaggcagcttgaagacgaaagggcctcgtgatacgcctatttttataggttaatgtcatgataataatggtttcttagacgtcaggtggcacttttcggggaaatgtgcgcggaccccctattggtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgtttttcaatgatgagcacttttaaagttctgctatgtgtcgcggtattatcccgtattgacgccgggcaagagcaactcggtcgccgcatacactattctcagaatgacttggtggtacctaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtacgaacggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcggtcagaccccgtagaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgataggtaccggataaggcgcagcggtcgggctgaacggggggttcgtgcacacagcccagcttggagcgaacgacctacaccgaactgagatacctacagcgcgagcattgagaaagcgccacgcttcccgaagggagaaaggcggacaggtatccggtaagcggcagggtcggaacaagagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcctttggctggccttttgctcacatgttctttcctgcgttatcccctgattctgtggataaccgtattaccgcctttgagtgagctgataccgctcgccgcagccgaacgaccgacggcgcagcgagtcagtgagcgaggaagcggaagagcgcccaatacgcaaaccgcctctccccgcgcgttggccgattcattaatgcagctgtggtgtcatggtcggtgatcgccagggtgccgacgcgcatctcgactgcatggtgcaccaatgcttctggcgtcaggcagccatcggaagctgtggtatggccgtgcaggtcgtaaatcactgcataattcgtgtcgctcaaggcgcactcccgttctggataatgttttttgcggcgacatcataacggttctggcaaatattctgaaatgagctggtgacaattaatcatcgaactagttaactagtacgcaagttcacgtaaaaagggtatcgcggaatt

SEQ ID NO: 83 - DNA, 57 bp, Unknown
  Plasmid pAP283-58 putative ribosomal binding site
tctagaattttctgcgcacccatcccgggtggcgcccaaagtgaggaaaatcacatg

SEQ ID NO: 84 - DNA, 35 bp, Unknown
  pAP283-58 Shine-Dalgarno sequence
tctagattaacccaacgcgtaggagtcaggccatg

SEQ ID NO: 85 - protein, 9 aa, Unknown
  Synthetic linker peptide
  REPEAT (1)..(1): May be absent or repeated up to 5 times
  REPEAT (3)..(3): May be absent or repeated up to 10 times
  REPEAT (4)..(4): May be absent or repeated up to 2 times
  REPEAT (5)..(9): May be absent or repeated up to 3 times
GCGSGGGGS

SEQ ID NO: 86 - protein, 10 aa, Unknown
  Synthetic linker peptide
  REPEAT (1)..(1): May be absent or repeated up to 10 times
  REPEAT (2)..(2): May be absent or repeated up to 2 times
  REPEAT (3)..(7): May be absent or repeated up to 3 times
  REPEAT (8)..(8): May be absent or repeated up to 8 times
  REPEAT (10)..(10): May be absent or repeated up to 5 times
GSGGGGSGCG

SEQ ID NO: 87 - protein, 5 aa, Unknown
  Synthetic linker peptide
  REPEAT (1)..(5): May be repeated
GGGGS

SEQ ID NO: 88 - protein, 10 aa, Unknown
  Synthetic N-terminal gamma1 linker peptide
CGDKTHTSPP

SEQ ID NO: 89 - protein, 10 aa, Unknown
  Synthetic C-terminal gamma 1 linker peptide
DKTHTSPPCG

SEQ ID NO: 90 - protein, 17 aa, Unknown
  Synthetic N-terminal gamma 3 linker peptide
CGGPKPSTPPGSSGGAP

SEQ ID NO: 91 - protein, 18 aa, Unknown
  Synthetic C-terminal gamma 3 linker peptide
PKPSTPPGSSGGAPGGCG

SEQ ID NO: 92 - protein, 6 aa, Unknown
  Synthetic N-terminal glycine linker peptide
GCGGGG

SEQ ID NO: 93 - protein, 6 aa, Unknown
  Synthetic C-terminal glycine linker peptide
GGGGCG

SEQ ID NO: 94 - protein, 6 aa, Unknown
  Synthetic N-terminal linker peptide
CGKKGG

SEQ ID NO: 95 - protein, 6 aa, Unknown
  Synthetic N-terminal linker peptide
CGDEGG

SEQ ID NO: 96 - protein, 6 aa, Unknown
  Synthetic C-terminal linker peptide
GGKKGC

SEQ ID NO: 97 - protein, 6 aa, Unknown
  Synthetic C-terminal linker peptide
GGEDGC

SEQ ID NO: 98 - protein, 4 aa, Unknown
  Synthetic linker peptide
GGCG

SEQ ID NO: 99 - protein, 9 aa, Homo sapiens
KTWGQYWQV

SEQ ID NO: 100 - protein, 9 aa, Homo sapiens
ITDQVPFSV

SEQ ID NO: 101 - protein, 9 aa, Homo sapiens
YLEPGPVTA

SEQ ID NO: 102 - protein, 10 aa, Homo sapiens
LLDGTATLRL

SEQ ID NO: 103 - protein, 10 aa, Homo sapiens
VLYRYGSFSV

SEQ ID NO: 104 - protein, 9 aa, Homo sapiens
AAGIGILTV

SEQ ID NO: 105 - protein, 9 aa, Homo sapiens
ILTVILGVL

SEQ ID NO: 106 - protein, 9 aa, Unknown
  Synthetic peptide
MLLAVLYCL

SEQ ID NO: 107 - protein, 9 aa, Unknown
  Synthetic peptide
YMDGTMSQV

SEQ ID NO: 108 - protein, 9 aa, Unknown
  Synthetic peptide
VLPDVFIRC

SEQ ID NO: 109 - protein, 9 aa, Unknown
  Synthetic peptide
FLWGPRALV

SEQ ID NO: 110 - protein, 9 aa, Homo sapiens
YLSGANLNL

SEQ ID NO: 111 - protein, 9 aa, Homo sapiens
RMPEAAPPV

SEQ ID NO: 112 - protein, 9 aa, Homo sapiens
STPPPGTRV

SEQ ID NO: 113 - protein, 9 aa, Homo sapiens
LLGRNSFEV

SEQ ID NO: 114 - protein, 9 aa, Homo sapiens
KIFGSLAFL

SEQ ID NO: 115 - protein, 9 aa, Homo sapiens
IISAVVGIL

SEQ ID NO: 116 - protein, 8 aa, Human papillomavirus type 16
TLGIVCPI

SEQ ID NO: 117 - DNA, 21 bp, Unknown
  Synthetic oligonucleotide
  misc_feature (1)..(21): Nucleotides are connected by phosphorothioate bonds
tccatgacgttcctgaataat

SEQ ID NO: 118 - DNA, 20 bp, Unknown
  Synthetic oligonucleotide
  misc_feature (1)..(20): Nucleotides are connected by phosphorothioate bonds
tccatgacgttcctgacgtt

SEQ ID NO: 119 - DNA, 19 bp, Unknown
  Synthetic oligonucleotide
  misc_feature (1)..(19): Nucleotides are connected by phosphorothioate bonds
ggggtcaacgttgaggggg

SEQ ID NO: 120 - DNA, 21 bp, Unknown
  Synthetic oligonucleotide
  misc_feature (1)..(21): Nucleotides are connected by phosphorothioate bonds
attattcaggaacgtcatgga

SEQ ID NO: 121 - DNA, 30 bp, Unknown
  Synthetic oligonucleotides
  misc_feature (1)..(30): Nucleotides are connected by phosphorothioate bonds
gggggggggggacgatcgtcgggggggggg

SEQ ID NO: 122 - DNA, 43 bp, Unknown
  Synthetic oligonucleotide
  misc_feature (1)..(43): Nucleotides are connected by phosphorothioate bonds
tccatgacgttcctgaataataaatgcatgtcaaagacagcat

SEQ ID NO: 123 - DNA, 63 bp, Unknown
  Synthetic oligonucleotide
  misc_feature (1)..(63): Nucleotides are connected by phosphorothioate bonds
tccatgacgttcctgaataattccatgacgttcctgaataattccatgacgttcctgaataat

SEQ ID NO: 124 - DNA, 61 bp, Unknown
  Synthetic oligonucleotide
  misc_feature (1)..(61): Nucleotides are connected by phosphorothioate bonds
tccatgacgttcctgaataatcgcgcgcgcgcgcgcgcgcgcgcgcgcgcgcgcgcgcgcg

SEQ ID NO: 125 - DNA, 23 bp, Unknown
  Synthetic oligonucleotide
  misc_feature (1)..(23): Nucleotides are connected by phosphorothioate bonds
tcgtcgttttgtcgttttgtcgt

SEQ ID NO: 126 - DNA, 20 bp, Unknown
  Synthetic oligonucleotide
  misc_feature (1)..(20): Nucleotides are connected by phosphorothioate bonds
ggttcttttggtccttgtct

SEQ ID NO: 127 - protein, 9 aa, Lymphocytic choriomeningitis virus
KAVYNFATM

SEQ ID NO: 128 - protein, 12 aa, Artificial Sequence
  N-terminal linked Lymphocytic choriomeningitis virus peptide
CGGKAVYNFATM

SEQ ID NO: 129 - protein, 12 aa, Artificial Sequence
  C-terminal linked Lymphocytic choriomeningitis virus peptide
KAVYNFATMGGC

SEQ ID NO: 130 - DNA, 21 bp, Unknown
  Synthetic oligonucleotide
tccatgacgttcctgaataat

SEQ ID NO: 131 - DNA, 20 bp, Unknown
  Synthetic oligonucleotide
tccatgacgttcctgacgtt

SEQ ID NO: 132 - DNA, 19 bp, Unknown
  Synthetic oligonucleotide
ggggtcaacgttgaggggg

SEQ ID NO: 133 - DNA, 21 bp, Unknown
  Synthetic oligonucleotide
attattcaggaacgtcatgga

SEQ ID NO: 134 - DNA, 30 bp, Unknown; identical to SEQ ID NO: 121
  Synthetic oligonucleotides

SEQ ID NO: 135 - DNA, 43 bp, Unknown; identical to SEQ ID NO: 122
  Synthetic oligonucleotide

SEQ ID NO: 136 - DNA, 63 bp, Unknown; identical to SEQ ID NO: 123
  Synthetic oligonucleotide

SEQ ID NO: 137 - DNA, 61 bp, Unknown; identical to SEQ ID NO: 124
  Synthetic oligonucleotide

SEQ ID NO: 138 - DNA, 23 bp, Unknown
  Synthetic oligonucleotide
tcgtcgttttgtcgttttgtcgt

SEQ ID NO: 139 - DNA, 19 bp, Unknown
  Synthetic oligonucleotide
ggggtcaacgttgaggggg

SEQ ID NO: 140 - DNA, 20 bp, Unknown
  Synthetic oligonucleotide
ggttcttttggtccttgtct

SEQ ID NO: 141 - skipped

SEQ ID NO: 142 - DNA, 594 bp, Hepatitis B virus
  CDS (1)..(594)
atggacattgacccttataaagaatttggagctactgtggagttactctcgtttttgccttctgacttctttccttccgtcagagatctcctagacaccgcctcagctctgtatcgagaagccttagagtctcctgagcattgctcacctcaccatactgcactcaggcaagccattctctgctggggggaattgatgactctagctacctgggtgggtaataatttggaagatccagcatccagggatctagtagtcaattatgttaatactaacatgggtttaaagatcaggcaactattgtggtttcatatatcttgccttacttttggaagagagactgtacttgaatatttggtctctttcggagtgtggattcgcactcctccagcctatagaccaccaaatgcccctatcttatcaacacttccggaaactactgttgttagacgacgggaccgaggcaggtcccctagaagaagaactccctcgcctcgcagacgcagatctcaatcgccgcgtcgcagaagatctcaatctcgggaatctcaatgtcttctccttaaagctgtttacaacttcgctaccatgtaa

SEQ ID NO: 143 - protein, 197 aa, Hepatitis B virus
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQCLLLKAVYNFATM

(19) United States
(12) Patent Application Publication
(10) Pub. No.: US 2015/0320855 A1
(43) Pub. Date: Nov. 12, 2015

(54) IN VIVO ACTIVATION OF ANTIGEN PRESENTING CELLS FOR ENHANCEMENT OF IMMUNE RESPONSES INDUCED BY VIRUS-LIKE PARTICLES

(71) Applicant: CYTOS BIOTECHNOLOGY AG, ZURICH-SCHLIEREN (CH)

(72) Inventors: MARTIN F. BACHMANN, SEUZACH (CH); FRANZISKA LECHNER, ZURICH (CH); TAZIO STORNI, VIGANELLO (CH)

(73) Assignee: CYTOS BIOTECHNOLOGY AG, ZURICH-SCHLIEREN (CH)

(21) Appl. No.: 14/567,945

(22) Filed: Dec. 11, 2014

Related U.S. Application Data
(63) Continuation of application No. 13/721,662, filed on Dec. 20, 2012, now abandoned.
Application No. 13/721,662 is a continuation of application No. 12/728,008, filed on Mar. 19, 2010, now abandoned.
Application No. 12/728,008 is a continuation of application No. 10/243,739, filed on Sep. 16, 2002, now abandoned.
(60) Provisional application No. 60/318,967, filed on Sep. 14, 2001.

(51) Int. Cl. A61K 39/29; A61K 39/39; C07K 14/005; A61K 39/12; C12N 7/00; A61K 39/385; A61K 39/00

(52) U.S. Cl. CPC A61K 39/292; A61K 39/385; A61K 39/39; A61K 39/0011; A61K 39/12; C12N 7/00; C07K 14/005; A61K 2039/5258; A61K 2039/6075; A61K 2039/55561; C12N 2760/10034; C12N 2730/10141; C12N 2730/10134; C12N 2730/10123; C07K 2319/00

(57) ABSTRACT

The invention relates to the finding that stimulation of antigen presenting cell (APC) activation using substances such as anti-CD40 antibodies or DNA oligomers rich in non-methylated C and G (CpGs) can dramatically enhance the specific T cell response obtained after vaccination with recombinant virus like particles (VLPs) coupled, fused or otherwise attached to antigens. While vaccination with recombinant VLPs fused to a cytotoxic T cell (CTL) epitope of lymphocytic choriomeningitis virus induced low levels cytolytic activity only and did not induce efficient anti-viral protection, VLPs injected together with anti-CD40 antibodies or CpGs induced strong CTL activity and full anti-viral protection. Thus, stimulation of APC-activation through antigen presenting cell activators such as anti-CD40 antibodies or CpGs can exhibit a potent adjuvant effect for vaccination with VLPs coupled, fused or attached otherwise to antigens.

### CROSS-REFERENCE TO RELATED APPLICATIONS

This application is a continuation of U.S. application Ser. No. 13/721,662, filed Dec. 20, 2012, which is a continuation of U.S. application Ser. No. 12/728,008, filed Mar. 19, 2010, which is a continuation of U.S. application Ser. No. 10/243,739, filed Sep. 16, 2002, which claims benefit of U.S. Provisional Application No. 60/318,967, filed Sep. 14, 2001, which is hereby incorporated by reference in its entirety.

### BACKGROUND OF THE INVENTION

1. Field of the Invention

The present invention is related to the fields of vaccinology, immunology, virology and medicine. The invention provides compositions and methods for enhancing T cell responses against antigens coupled, fused or otherwise attached to virus-like particles (VLPs) by stimulating the innate immune system, in particular by activating antigen presenting cells (APCs), using substances such as anti-CD40 antibodies or immunostimulatory nucleic acids, in particular DNA oligomers rich in non-methylated cytosine and guanine (CpGs). The invention can be used to induce strong and sustained T cell responses particularly useful for the treatment of tumors and chronic viral diseases.

2. Related Art

The essence of the immune system is built on two separate foundation pillars: one is specific or adaptive immunity which is characterized by relatively slow response-kinetics and the ability to remember; the other is non-specific or innate immunity exhibiting rapid response-kinetics but lacking memory. Lymphocytes are the key players of the adaptive immune system. Each lymphocyte expresses antigen-receptors of unique specificity. Upon recognizing an antigen via the receptor, lymphocytes proliferate and develop effector function. Few lymphocytes exhibit specificity for a given antigen or pathogen, and massive proliferation is usually required before an effector response can be measured—hence, the slow kinetics of the adaptive immune system. Since a significant proportion of the expanded lymphocytes survive and may maintain some effector function following elimination of the antigen, the adaptive immune system reacts faster when encountering the antigen a second time. This is the basis of its ability to remember.

In contrast to the situation with lymphocytes, where specificity for a pathogen is confined to few cells that must expand to gain function, the cells and molecules of the innate immune system are usually present in massive numbers and recognize a limited number of invariant features associated with pathogens (Medzhitov, R. and Janeway, C. A., Jr., Cell 91:295-298 (1997)). Examples of such patterns include lipopolysaccharides (LPS), non-methylated CG-rich DNA (CpG) or double stranded RNA, which are specific for bacterial and viral infections, respectively.

Most research in immunology has focused on the adaptive immune system and only recently has the innate immune system entered the focus of interest. Historically, the adaptive and innate immune system were treated and analyzed as two separate entities that had little in common. Such was the disparity that few researchers wondered why antigens were much more immunogenic for the specific immune system when applied with adjuvants that stimulated innate immunity (Sotomayor, E. M., et al., Nat. Med. 5:780 (1999); Diehl, L., et al., Nat. Med. 5:774 (1999); Weigle, W. O., Adv. Immunol. 30:159 (1980)). However, the answer posed by this question is critical to the understanding of the immune system and for comprehending the balance between protective immunity and autoimmunity.

Rationalized manipulation of the innate immune system and in particular activation of APCs involved in T cell priming to deliberately induce a self-specific T cell response provides a means for T cell-based tumor-therapy. Accordingly, the focus of most current therapies is on the use of activated dendritic cells (DCs) as antigen-carriers for the induction of sustained T cell responses (Nestle et al., Nat. Med. 4:328 (1998)). Similarly, in vivo activators of the innate immune system, such as CpGs or anti-CD40 antibodies, are applied together with tumor cells in order to enhance their immunogenicity (Sotomayor, E. M., et al., Nat. Med. 5:780 (1999); Diehl, L., et al., Nat. Med. 5:774 (1999)).

Generalized activation of APCs by factors that stimulate innate immunity may often be the cause for triggering self-specific lymphocytes and autoimmunity. Activation may result in enhanced expression of costimulatory molecules or cytokines such as IL-12 or IFN-α. This view is compatible with the observation that administration of LPS together with thyroid extracts is able to overcome tolerance and trigger autoimmune thyroiditis (Weigle, W. O., Adv. Immunol. 30:159 (1980)). Moreover, in a transgenic mouse model, it was recently shown that administration of self-peptide alone failed to cause auto-immunity unless APCs were activated by a separate pathway (Garza, K. M., et al., J. Exp. Med. 191:2021 (2000)). The link between innate immunity and autoimmune disease is further underscored by the observation that LPS, viral infections or generalized activation of APCs delays or prevents the establishment of peripheral tolerance (Vella, A. T., et al., Immunity 2:261 (1995); Ehl, S., et al., J. Exp. Med. 187:763 (1998); Maxwell, J. R., et al., J. Immunol. 162:2024 (1999)). In this way, innate immunity not only enhances the activation of self-specific lymphocytes but also inhibits their subsequent elimination.

Induction of cytotoxic T lymphocyte (CTL) responses after immunization with minor histocompatibility antigens, such as the HY-antigen, requires the presence of T helper cells (Th cells) (Husmann, L. A., and M. J. Bevan, Ann. NY. Acad. Sci. 532:158 (1988); Guerder, S., and P. Matzinger, J. Exp. Med. 176:553 (1992)). CTL-responses induced by cross-priming, i.e. by priming with exogenous antigens that reached the class I pathway, have also been shown to require the presence of Th cells (Bennett, S. R. M., et al., J. Exp. Med. 186:65 (1997)). These observations have important consequences for tumor therapy where T help may be critical for the induction of protective CTL responses by tumor cells (Ossendorp, F., et al., J. Exp. Med. 187:693 (1998)).

An important effector molecule on activated Th cells is the CD40-ligand (CD40L) interacting with CD40 on B cells, macrophages and dendritic cells (DCs) (Foy, T. M., et al., Annu. Rev. Immunol. 14:591 (1996)). Triggering of CD40 on B cells is essential for isotype switching and the generation of B cell memory (Foy, T. M., et al., Ann. Rev. Immunol. 14:591 (1996)). More recently, it was shown that stimulation of CD40 on macrophages and DCs leads to their activation and maturation (Cella, M., et al., Curr. Opin. Immunol. 9:10 (1997); Banchereau, J., and R. M. Steinman Nature 392:245 (1998)). Specifically, DCs upregulate costimulatory molecules and produce cytokines such as IL-12 upon activation. Interestingly, this CD40L-mediated maturation of DCs seems to be responsible for the helper effect on CTL responses. In fact, it has recently been shown that CD40-triggering by Th cells renders DCs able to initiate a CTL-response (Ridge, J. P., et al., Nature 393:474 (1998); Bennett, S. R. M., et al., Nature 393:478 (1998); Schoenenberger, S. P., et al., Nature 393:480 (1998)). This is consistent with the earlier observation that Th cells have to recognize their ligands on the same APC as the CTLs, indicating that a cognate interaction is required (Bennett, S. R. M., et al., J. Exp. Med. 186:65 (1997)). Thus CD40L-mediated stimulation by Th cells leads to the activation of DCs, which subsequently are able to prime CTL-responses. In the human, type I interferons, in particular interferon α and β may be equally important as IL-12.

In contrast to these Th-dependent CTL responses, viruses are often able to induce protective CTL-responses in the absence of T help (for review, see (Bachmann, M. F., et al., J. Immunol. 161:5791 (1998)). Specifically, lymphocytic choriomeningitis virus (LCMV) (Leist, T. P., et al., J. Immunol. 138:2278 (1987); Ahmed, R., et al., J. Virol. 62:2102 (1988); Battegay, M., et al., Cell Immunol. 167:115 (1996); Borrow, P., et al., J. Exp. Med. 183:2129 (1996); Whitmire, J. K., et al., J. Virol. 70:8375 (1996)), vesicular stomatitis virus (VSV) (Kündig, T. M., et al., Immunity 5:41 (1996)), influenza virus (Tripp, R. A., et al., J. Immunol. 155:2955 (1995)), vaccinia virus (Leist, T. P., et al., Scand. J. Immunol. 30:679 (1989)) and ectromelia virus (Buller, R., et al., Nature 328:77 (1987)) were able to prime CTL-responses in mice depleted of CD4⁺ T cells or deficient for the expression of class II or CD40. The mechanism for this Th cell independent CTL-priming by viruses is presently not understood. Moreover, most viruses do not stimulate completely Th cell independent CTL-responses, but virus-specific CTL-activity is reduced in Th-cell deficient mice. Thus, Th cells may enhance anti-viral CTL-responses but the mechanism of this help is not fully understood yet. DCs have recently been shown to present influenza derived antigens by cross-priming (Albert, M. L., et al., J. Exp. Med. 188:1359 (1998); Albert, M. L., et al., Nature 392:86 (1998)). It is therefore possible that, similarly as shown for minor histocompatibility antigens and tumor antigens (Ridge, J. P., et al., Nature 393:474 (1998); Bennett, S. R. M., et al., Nature 393:478 (1998); Schoenenberger, S. P., et al., Nature 393:480 (1998)), Th cells may assist induction of CTLs via CD40 triggering on DCs. Thus, stimulation of CD40 using CD40L or anti-CD40 antibodies may enhance CTL induction after stimulation with viruses or tumor cells.

However, although CD40L is an important activator of DCs, there seem to be additional molecules that can stimulate maturation and activation of DCs during immune responses. In fact, CD40 is not measurably involved in the induction of CTLs specific for LCMV or VSV (Ruedl, C., et al., J. Exp. Med. 189:1875 (1999)). Thus, although VSV-specific CTL responses are partly dependent upon the presence of CD4⁺T cells (Kündig, T. M., et al., Immunity 5:41 (1996)), this helper effect is not mediated by CD40L. Candidates for effector molecules triggering maturation of DCs during immune responses include Trance and TNF (Bachmann, M. F., et al., J. Exp. Med. 189:1025 (1999); Sallusto, F., and A. Lanzavecchia, J. Exp. Med. 179:1109 (1994)), but it is likely that there are more proteins with similar properties such as, e.g., CpGs.

It is well established that the administration of purified proteins alone is usually not sufficient to elicit a strong immune response; isolated antigen generally must be given together with helper substances called adjuvants. Within these adjuvants, the administered antigen is protected against rapid degradation, and the adjuvant provides an extended release of a low level of antigen.

Unlike isolated proteins, viruses induce prompt and efficient immune responses in the absence of any adjuvants both with and without T-cell help (Bachmann & Zinkernagel, Ann. Rev. Immunol. 15:235-270 (1997)). Although viruses often consist of few proteins, they are able to trigger much stronger immune responses than their isolated components. For B cell responses, it is known that one crucial factor for the immunogenicity of viruses is the repetitiveness and order of surface epitopes. Many viruses exhibit a quasi-crystalline surface that displays a regular array of epitopes which efficiently crosslinks epitope-specific immunoglobulins on B cells (Bachmann & Zinkernagel, Immunol. Today 17:553-558 (1996)). This crosslinking of surface immunoglobulins on B cells is a strong activation signal that directly induces cell-cycle progression and the production of IgM antibodies. Further, such triggered B cells are able to activate T helper cells, which in turn induce a switch from IgM to IgG antibody production in B cells and the generation of long-lived B cell memory—the goal of any vaccination (Bachmann & Zinkernagel, Ann. Rev. Immunol. 15:235-270 (1997)). Viral structure is even linked to the generation of anti-antibodies in autoimmune disease and as a part of the natural response to pathogens (see Fehr, T., et al., J. Exp. Med. 185:1785-1792 (1997)). Thus, antigens on viral particles that are organized in an ordered and repetitive array are highly immunogenic since they can directly activate B cells.

In addition to strong B cell responses, viral particles are also able to induce the generation of a cytotoxic T cell response, another crucial arm of the immune system. These cytotoxic T cells are particularly important for the elimination of non-cytopathic viruses such as HIV or Hepatitis B virus and for the eradication of tumors. Cytotoxic T cells do not recognize native antigens but rather recognize their degradation products in association with MHC class I molecules (Townsend & Bodmer, Ann. Rev. Immunol. 7:601-624 (1989)). Macrophages and dendritic cells are able to take up and process exogenous viral particles (but not their soluble, isolated components) and present the generated degradation product to cytotoxic T cells, leading to their activation and proliferation (Kovacsovics-Bankowski et al., Proc. Nati. Acad. Sci. USA 90:4942-4946 (1993); Bachmann et al., Eur. J. Immunol. 26:2595-2600 (1996)).

Viral particles as antigens exhibit two advantages over their isolated components: (1) due to their highly repetitive surface structure, they are able to directly activate B cells, leading to high antibody titers and long-lasting B cell memory; and (2) viral particles but not soluble proteins are able to induce a cytotoxic T cell response, even if the viruses are non-infectious and adjuvants are absent.

Several new vaccine strategies exploit the inherent immunogenicity of viruses. Some of these approaches focus on the particulate nature of the virus particle; for example see Harding, C. V. and Song, R., (J. Immunology 153:4925 (1994)), which discloses a vaccine consisting of latex beads and antigen; Kovacsovics-Bankowski, M., et al. (Proc. Natl. Acad. Sci. USA 90:4942-4946 (1993)), which discloses a vaccine consisting of iron oxide beads and antigen; U.S. Pat. No. 5,334,394 to Kossovsky, N., et al., which discloses core particles coated with antigen; U.S. Pat. No. 5,871,747, which discloses synthetic polymer particles carrying on the surface one or more proteins covalently bonded thereto; and a core particle with a non-covalently bound coating, which at least partially covers the surface of said core particle, and at least one biologically active agent in contact with said coated core particle (see, e.g., WO 94/15585).

In a further development, virus-like particles (VLPs) are being exploited in the area of vaccine production because of both their structural properties and their non-infectious nature. VLPs are supermolecular structures built in a symmetric manner from many protein molecules of one or more types. They lack the viral genome and, therefore, are noninfectious. VLPs can often be produced in large quantities by heterologous expression and can be easily be purified.

There have been remarkable advances made in vaccination strategies recently, yet there remains a need for improvement on existing strategies. In particular, there remains a need in the art for the development of new and improved vaccines that promote a strong CTL immune response and anti-pathogenic protection as efficiently as natural pathogens.

### SUMMARY OF THE INVENTION

This invention is based on the surprising finding that in vivo stimulation of APC-activation, resulting in enhanced expression of costimulatory molecules or cytokines, increases T cell responses induced by antigens coupled, fused or otherwise attached to VLPs or induced by the VLP its elf.

Also unexpectedly, stimulation of innate immunity was more efficient at enhancing CTL responses induced by these modified VLPs than CTL responses induced by free peptide. The technology allows for the creation of highly efficient vaccines against infectious diseases as well as for the creation of vaccines for the treatment of cancers.

In a first embodiment, the invention provides a composition for enhancing an immune response against an antigen in an animal comprising a virus-like particle coupled, fused or otherwise attached, i.e., bound, to an antigen, which virus-like particle bound to said antigen is capable of inducing an immune response against the antigen in the animal and a substance that activates antigen presenting cells in an amount sufficient to enhance the immune response of the animal to the antigen.

In another embodiment, the invention provides a composition for enhancing an immune response against a virus-like particle in an animal comprising a virus-like particle capable of being recognized by the immune system of the animal and/or inducing an immune response against the virus-like particle in the animal and at least one substance that activates antigen presenting cells in an amount sufficient to enhance the immune response of the animal to the virus-like particle. In this embodiment, the virus-like particle is the antigen to which an immune response is desired and an immune response is induced by the virus-like particle itself, which is then enhanced by the APC-activating substance.

In a preferred embodiment, the virus-like particle is a recombinant virus-like particle. Also preferred, the virus-like particle is free of a lipoprotein envelope. Preferably, the recombinant virus-like particle comprises, or alternatively consists of, recombinant proteins of Hepatitis B virus, measles virus, Sindbis virus, Rotavirus, Foot-and-Mouth-Disease virus, Retrovirus, Norwalk virus or human Papilloma virus, RNA-phages, Qβ-phage, GA-phage, fr-phage, AP205 phage and Ty. In a specific embodiment, the virus-like particle comprises, or alternatively consists of, one or more different Hepatitis B virus core (capsid) proteins (HBcAgs). In a further specific embodiment, the virus-like particle comprises, or alternatively consists of, one or more different Qβ coat proteins.

In another embodiment, the antigen is a recombinant antigen. In yet another embodiment, the antigen can be selected from the group consisting of: (1) a polypeptide suited to induce an immune response against cancer cells; (2) a polypeptide suited to induce an immune response against infectious diseases; (3) a polypeptide suited to induce an immune response against allergens; (4) a polypeptide suited to induce an improved response against self-antigens; and (5) a polypeptide suited to induce an immune response in farm animals or pets.

In yet another embodiment, the antigen can be selected from the group consisting of: (1) an organic molecule suited to induce an immune response against cancer cells; (2) an organic molecule suited to induce an immune response against infectious diseases; (3) an organic molecule suited to induce an immune response against allergens; (4) an organic molecule suited to induce an improved response against self-antigens; (5) an organic molecule suited to induce an immune response in farm animals or pets; and (6) an organic molecule suited to induce a response against a drug, a hormone or a toxic compound.

In a particular embodiment, the antigen comprises, or alternatively consists of, a cytotoxic T cell epitope. In a related embodiment, the virus-like particle comprises the Hepatitis B virus core protein and the cytotoxic T cell epitope is fused to the C-terminus of said Hepatitis B virus core protein. In one embodiment, they are fused by a linking sequence. In a related embodiment, the virus-like particle comprises the Qβ coat protein and the cytotoxic T cell epitope is fused to said Qβ coat protein. In one embodiment, they are fused by a linking sequence. In a related embodiment, the virus-like particle comprises the Qβ coat protein and the cytotoxic T cell epitope is coupled to said Qβ coat protein.

In another aspect of the invention the composition comprises a substance that activates antigen presenting cells. In one embodiment, the substance stimulates upregulation of costimulatory molecules on antigen presenting cells and/or prolong their survival. In another embodiment, the substance induces nuclear translocation of NF-κB in antigen presenting cells, preferably dendritic cells. In yet another embodiment, the substance activates toll-like receptors in antigen presenting cells.

In a particular embodiment, the substance comprises, or alternatively consists of, a substance that activates CD40, such as anti-CD40 antibodies, and/or immunostimulatory nucleic acids, in particular DNA oligomers containing unmethylated cytosine and guanine (CpGs).

In another aspect of the invention, there is provided a method of enhancing an immune response against an antigen in a human or other animal species comprising introducing into the animal a virus-like particle coupled, fused or otherwise attached to at least one antigen, which virus-like particle bound to the at least one antigen, i.e. the “modified virus-like particle” as used herein, is capable of inducing an immune response against the antigen in the animal, and at least one substance that activates antigen presenting cells in an amount sufficient to enhance the immune response of the animal to the antigen.

In one embodiment, the virus-like particle coupled, fused or otherwise attached to an antigen and the substance that activates antigen presenting cells are introduced into the human or animal subject successively, whereas in another embodiment they are introduced simultaneously.

In yet another embodiment of the invention, the virus-like particle coupled, fused or otherwise attached to an antigen and the substance that activates antigen presenting cells are introduced into an animal subcutaneously, intramuscularly, intranasally, intradermally, intravenously or directly into a lymph node. In an equally preferred embodiment, the immune enhancing composition is applied locally, near a tumor or local viral reservoir against which one would like to vaccinate.

In an equally preferred embodiment, the immune response is sought to be directed against the virus-like particle itself, e.g. against the Hepatitis B virus core protein. To this purpose, the virus-like particle and the substance that activates antigen presenting cells are introduced into an animal subcutaneously, intramuscularly, intranasally, intradermally, intravenously or directly into a lymph node. In an equally preferred embodiment, the immune enhancing composition is applied locally, near a tumor or local viral reservoir against which one would like to vaccinate.

In a preferred aspect of the invention, the immune response is a T cell response, and the T cell response against the antigen is enhanced. In a specific embodiment, the T cell response is a cytotoxic T cell response, and the cytotoxic T cell response against the antigen is enhanced.

The present invention also relates to a vaccine comprising an immunologically effective amount of the immune response enhancing compositions of the present invention together with a pharmaceutically acceptable diluent, carrier or excipient. In a preferred embodiment, the vaccine further comprises at least one adjuvant, such as incomplete Freund's adjuvant. The invention also provides a method of immunizing and/or treating an animal comprising administering to the animal an immunologically effective amount of the disclosed vaccine.

The invention further provides a method of enhancing anti-viral protection in an animal comprising introducing into the animal the compositions of the invention.

It is to be understood that both the foregoing general description and the following detailed description are exemplary and explanatory only and are intended to provide further explanation of the invention as claimed.

### BRIEF DESCRIPTION OF THE DRAWINGS/FIGURES

FIG. 1 shows the DNA sequence of the HBcAg containing peptide p33 from lymphocytic choriomeningitis virus (p33-VLPs). The nonameric p33 epitope is genetically fused to the C-terminus of the hepatitis B core protein at position 183 via a three leucine linking sequence.

FIG. 2 shows the structure of the p33-VLPs as assessed by electron microscopy (A) and SDS PAGE (B). Recombinantly produced wild-type VLPs (composed of HBcAg[aa.1-183]monomers) and p33-VLPs were loaded onto a Sephacryl S-400 gel filtration column (Amersham Pharmacia Biotechnology AG) for purification. Pooled fractions were loaded onto a Hydroxyapatite column. Flow through (which contains purified HBc capsids) was collected and loaded onto a reducing SDS-PAGE gel for monomer molecular weight analysis (B).

FIG. 3 shows that VLP-derived p33 is processed by DCs and presented in association with MHC class I. Various cells (DCs, inclusive CD8⁺ and CD8⁻ subsets, B and T cells) were pulsed with p33-VLPs, VLP and p33 peptide for 1 hour. After three washings, presenter cells (10⁴) were co-cultured with CD8⁺ T cells specific for p33 (33) (10⁵) for 2 days. The proliferation was assayed by measurement of thymidine incorporation (DCs (black bars), B cells (white bars) and T cells (grey bars)).

FIG. 4 shows that VLP-derived p33 is processed by macrophages and presented in association with MHC class I. DCs and macrophages were pulsed with p33-VLPs, VLP and p33 peptide for 1 hour. After three washings, presenter cells (10⁴) were co-cultured with CD8⁺ antigen-specific T cells (Pircher, H. P., et al., Nature 342:559 (1989)) (10⁵) for 2 days. The proliferation was assayed by measurement of thymidine incorporation (DCs (black bars) and peritoneal macrophages (white bars)).

FIG. 5 shows that anti-CD40 antibodies applied together with p33-VLPs dramatically enhance CTL activity specific for p33. C57BL/6 mice were primed with 100 μg p33-VLP alone (B) or in combination with 100 μg anti-CD40 antibodies (A). Spleens were removed after 10 days and restimulated for 5 days in vitro with p33-pulsed naïve splenocytes. CTL activity was tested in a classical 5 h-⁵¹Cr release assay using p33 labeled (filled circles) or unlabelled (open circles) EL-4 cells as target cells. Results were confirmed in two independent experiments.

FIG. 6 shows that anti-CD40 antibodies applied together with p33-VLPs dramatically enhance CTL activity specific for p33 if measured directly ex vivo. Mice were primed with 100 μg p33-VLP alone (B) or in combination with 100 μg anti-CD40 antibodies (A). Spleens were removed after 9 days and CTL activity was tested in a 5 h-⁵¹Cr release assay using p33 labeled (filled circles) or unlabelled (open circles) EL-4 cells as target cells.

FIG. 7 shows that CpGs applied together with p33-VLPs dramatically enhance CTL activity specific for p33 if measured after in vitro restimulation of CTLs. Mice were primed with 100 μg p33-VLP alone (B) or in combination with 20 nmol CpG (A). Spleens were removed after 10 days and restimulated for 5 days in vitro with p33-pulsed naïve splenocytes in presence of recombinant IL-2 (2 ng/well). CTL activity was tested in a classical 5 h-⁵¹Cr release assay using p33 labeled (filled boxes) or unlabelled (open boxes) EL-4 cells as target cells. Results were confirmed in two independent experiments.

FIG. 8 shows that CpGs applied together with p33-VLPs dramatically enhance CTL activity specific for p33 if measured directly ex vivo. Mice were primed with 100 μg p33-VLP alone (B) or in combination with 20 nmol CpG DNA (A). Spleens were removed after 9 days and CTL activity was tested in a 5 h-⁵¹Cr release assay using p33 labeled (filled circles) or unlabelled (open circles) EL-4 cells as target cells.

FIG. 9 shows that anti-CD40 antibodies are more efficient at enhancing CTL responses against p33-VLPs than free p33. Mice were primed with 100 μg p33-VLP (A) or 100 μg p33 (B) in combination with 100 μg anti-CD40 antibodies. Spleens were removed after 9 days and CTL activity was tested in a 5 h-⁵¹Cr release assay using p33 labeled (filled circles) or unlabelled (open circles) EL-4 cells as target cells.

FIG. 10 shows that anti-CD40 antibodies applied together with p33-VLPs dramatically enhance anti-viral protection. Mice were primed intravenously with 100 μg of p33-VLPs alone or together with 100 μg of anti-CD40 antibodies. Twelve days later, mice were challenged with LCMV (200 pfu, intravenously) and viral titers were assessed in the spleen 4 days later as described in Bachmann, M. F., “Evaluation of lymphocytic choriomeningitis virus-specific cytotoxic T cell responses,” in Immunology Methods Manual, Lefkowitz, I., ed., Academic Press Ltd., New York, N.Y. (1997) p. 1921.

FIG. 11 shows that CpGs applied together with p33-VLPs dramatically enhance anti-viral protection. Mice were primed subcutaneously with 100 μg of p33-VLPs alone or together with 20 nmol CpGs. Twelve days later, mice were challenged with LCMV (200 pfu, intravenously) and viral titers were assessed in the spleen 4 days later as described in Bachmann, M. F., “Evaluation of lymphocytic choriomeningitis virus-specific cytotoxic T cell responses,” in Immunology Methods Manual, Lefkowitz, I., ed., Academic Press Ltd., New York, N.Y. (1997) p. 1921.

FIG. 12 shows that anti-CD40 antibodies or CpGs applied together with p33-VLPs dramatically enhance anti-viral protection. Mice were primed either subcutaneously or intradermally with 100 μg of p33-VLPs alone, or subcutaneously together with 20 nmol CpGs, or intravenously together with 100 μg of anti-CD40 antibodies. As a control, free peptide p33 (100 μg) was injected subcutaneously in IFA. Twelve days later, mice were challenged intraperitoneally with recombinant vaccinia virus expressing LCMV glycoprotein (1.5×10⁶ pfu) and viral titers were assessed in the ovaries 5 days later as described in Bachmann et al. “Evaluation of lymphocytic choriomeningitis virus-specific cytotoxic T cell responses” in Immunology Methods Manual, Lefkowitz, I., ed. Academic Press Ltd., New York N.Y. (1997) p. 1921.

FIG. 13 shows immunostimulatory nucleic acids mixed with VLPs coupled to antigen are strong adjuvants for induction of viral protection.

FIG. 14 shows different immunostimulatory nucleic acids mixed with a fusion protein of HBcAg VLPs with antigen induce a potent antigen-specific CTL response and virus protection.

FIG. 15 shows different immunostimulatory nucleic acids mixed with a fusion protein of HBcAg VLPs with antigen induce a potent antigen-specific CTL response and virus protection.

FIG. 16 shows the immunostimulatory nucleic acid G10pt mixed with VLP fusion protein or VLP coupled with antigen induces a potent antigen-specific CTL response and virus protection.

FIG. 17 shows immunostimulatory nucleic acids mixed with Qβ VLPs coupled to antigen are strong adjuvants for induction of viral protection.

FIG. 18 shows different immunostimulatory nucleic acids mixed with Qβ VLPs coupled to antigen induce a potent antigen-specific CTL response and virus protection.

FIG. 19 shows immunostimulatory nucleic acids mixed with AP205 VLPs coupled to antigen are strong adjuvants for induction of viral protection.

Table 1 shows anti-CD40 antibodies and CpG trigger maturation of dendritic cells. Dendritic cells were stimulated overnight with anti-CD40 antibodies (10 μg/well) or CpG (2 nmol/well) and expression of B7-1 and B7-2 was assessed by flow cytometry.

### DETAILED DESCRIPTION OF THE INVENTION

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are hereinafter described.

### 1. Definitions

Amino acid linker: An “amino acid linker”, or also just termed “linker” within this specification, as used herein, either associates the antigen or antigenic determinant with the second attachment site, or more preferably, already comprises or contains the second attachment site, typically—but not necessarily—as one amino acid residue, preferably as a cysteine residue. The term “amino acid linker” as used herein, however, does not intend to imply that such an amino acid linker consists exclusively of amino acid residues, even if an amino acid linker consisting of amino acid residues is a preferred embodiment of the present invention. The amino acid residues of the amino acid linker are, preferably, composed of naturally occurring amino acids or unnatural amino acids known in the art, all-L or all-D or mixtures thereof. However, an amino acid linker comprising a molecule with a sulfhydryl group or cysteine residue is also encompassed within the invention. Such a molecule comprise preferably a C1-C6 alkyl-, cycloalkyl (C5,C6), aryl or heteroaryl moiety. However, in addition to an amino acid linker, a linker comprising preferably a C1-C6 alkyl-, cycloalkyl- (C5,C6), aryl- or heteroaryl-moiety and devoid of any amino acid(s) shall also be encompassed within the scope of the invention. Association between the antigen or antigenic determinant or optionally the second attachment site and the amino acid linker is preferably by way of at least one covalent bond, more preferably by way of at least one peptide bond.

Animal: As used herein, the term “animal” taken to include, for example, humans, sheep, horses, cattle, pigs, dogs, cats, rats, mice, mammals, birds, reptiles, fish, insects and arachnids.

Antibody: As used herein, the term “antibody” refers to molecules which are capable of binding an epitope or antigenic determinant. The term is meant to include whole antibodies and antigen-binding fragments thereof, including single-chain antibodies. Most preferably the antibodies are human antigen binding antibody fragments and include, but are not limited to, Fab, Fab′ and F(ab′)2, Fd, single-chain Fvs (scFv), single-chain antibodies, disulfide-linked Fvs (sdFv) and fragments comprising either a V_{L} or V_{H} domain. The antibodies can be from any animal origin including birds and mammals. Preferably, the antibodies are human, murine, rabbit, goat, guinea pig, camel, horse or chicken. As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described, for example, in U.S. Pat. No. 5,939,598 by Kucherlapati et al.

Antigen: As used herein, the term “antigen” refers to a molecule capable of being bound by an antibody or a T cell receptor (TCR) if presented by MHC molecules. The term “antigen”, as used herein, also encompasses T-cell epitopes. An antigen is additionally capable of being recognized by the immune system and/or capable of inducing a humoral immune response and/or a cellular immune response leading to the activation of B- and/or T-lymphocytes. This may, however, require that, at least in certain cases, the antigen contains or is linked to a Th cell epitope and is given in adjuvant. An antigen can also have one or more epitopes (B- and T-epitopes). The specific reaction referred to above is meant to indicate that the antigen will preferably react, typically in a highly selective manner, with its corresponding antibody or TCR and not with the multitude of other antibodies or TCRs which may be evoked by other antigens.

A “microbial antigen” as used herein is an antigen of a microorganism and includes, but is not limited to, infectious virus, infectious bacteria, parasites and infectious fungi. Such antigens include the intact microorganism as well as natural isolates and fragments or derivatives thereof and also synthetic or recombinant compounds which are identical to or similar to natural microorganism antigens and induce an immune response specific for that microorganism. A compound is similar to a natural microorganism antigen if it induces an immune response (humoral and/or cellular) to a natural microorganism antigen. Such antigens are used routinely in the art and are well known to the skilled artisan.

Examples of infectious viruses that have been found in humans include but are not limited to: Retroviridae (e.g. human immunodeficiency viruses, such as HIV-1 (also referred to as HTLV-III, LAV or HTLV-III/LAV, or HIV-III); and other isolates, such as HIV-LP); Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus); Poxviridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Both gram negative and gram positive bacteria serve as antigens in vertebrate animals. Such gram positive bacteria include, but are not limited to, Pasteurella species, Staphylococci species and Streptococcus species. Gram negative bacteria include, but are not limited to, Escherichia coli, Pseudomonas species, and Salmonella species. Specific examples of infectious bacteria include but are not limited to: Helicobacter pyloris, Borelia burgdorferi, Legionella pneumophilia, Mycobacteria sps. (e.g. M. tuberculosis, M avium, M intracellulare, M. kansaii, M gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, Actinomyces israelli and Chlamydia.

Examples of infectious fungi include: Cryptococcus neoformans, Histoplasma capsulatum, Coccidioides immitis, Blastomyces dermatitidis, Chlamydia trachomatis and Candida albicans. Other infectious organisms (i.e., protists) include: Plasmodium such as Plasmodium falciparum, Plasmodium malariae, Plasmodium ovale, Plasmodium vivax, Toxoplasma gondii and Shistosoma.

Other medically relevant microorganisms have been descried extensively in the literature, e.g., see C. G. A. Thomas, “Medical Microbiology”, Bailliere Tindall, Great Britain 1983, the entire contents of which is hereby incorporated by reference.

The compositions and methods of the invention are also useful for treating cancer by stimulating an antigen-specific immune response against a cancer antigen. A “tumor antigen” as used herein is a compound, such as a peptide, associated with a tumor or cancer and which is capable of provoking an immune response, in particular, when presented in the context of an MHC molecule. Tumor antigens can be prepared from cancer cells either by preparing crude extracts of cancer cells, for example, as described in Cohen, et al., Cancer Research, 54:1055 (1994), by partially purifying the antigens, by recombinant technology or by de novo synthesis of known antigens. Tumor antigens include antigens that are antigenic portions of or are a whole tumor or cancer polypeptide. Such antigens can be isolated or prepared recombinantly or by any other means known in the art. Cancers or tumors include, but are not limited to, biliary tract cancer; brain cancer; breast cancer; cervical cancer; choriocarcinoma; colon cancer; endometrial cancer; esophageal cancer; gastric cancer; intraepithelial neoplasms; lymphomas; liver cancer; lung cancer (e.g. small cell and non-small cell); melanoma; neuroblastomas; oral cancer; ovarian cancer; pancreas cancer; prostate cancer; rectal cancer; sarcomas; skin cancer; testicular cancer; thyroid cancer; and renal cancer, as well as other carcinomas and sarcomas.

Antigenic determinant: As used herein, the term “antigenic determinant” is meant to refer to that portion of an antigen that is specifically recognized by either B- or T-lymphocytes. B-lymphocytes respond to foreign antigenic determinants via antibody production, whereas T-lymphocytes are the mediator of cellular immunity. Thus, antigenic determinants or epitopes are those parts of an antigen that are recognized by antibodies, or in the context of an MHC, by T-cell receptors.

Antigen presenting cell: As used herein, the term “antigen presenting cell” is meant to refer to a heterogenous population of leucocytes or bone marrow derived cells which possess an immunostimulatory capacity. For example, these cells are capable of generating peptides bound to MHC molecules that can be recognized by T cells. The term is synonymous with the term “accessory cell” and includes, for example, Langerhans' cells, interdigitating cells, B cells, macrophages, dendritic cells and also NK cells. Under some conditions, epithetral cells, endothelial cells and other non-bone marrow derived cells can also serve as antigen presenting cells. Activated APCs refers to APCs with a enhanced potential to stimulate T cells. This may be due to enhanced expression of costimulatory molecules or may be due to increased expression of cytokines such as IL-12 or interferons, chemokines or other secreted immunostimulatory molecules.

Association: As used herein, the term “association” as it applies to the first and second attachment sites, refers to the binding of the first and second attachment sites that is preferably by way of at least one non-peptide bond. The nature of the association may be covalent, ionic, hydrophobic, polar or any combination thereof, preferably the nature of the association is covalent.

Attachment Site, First: As used herein, the phrase “first attachment site” refers to an element of non-natural or natural origin, to which the second attachment site located on the antigen or antigenic determinant may associate. The first attachment site may be a protein, a polypeptide, an amino acid, a peptide, a sugar, a polynucleotide, a natural or synthetic polymer, a secondary metabolite or compound (biotin, fluorescein, retinol, digoxigenin, metal ions, phenylmethylsulfonylfluoride), or a combination thereof, or a chemically reactive group thereof. The first attachment site is located, typically and preferably on the surface, of the virus-like particle. Multiple first attachment sites are present on the surface of virus-like particle typically in a repetitive configuration.

Attachment Site, Second: As used herein, the phrase “second attachment site” refers to an element associated with the antigen or antigenic determinant to which the first attachment site located on the surface of the virus-like particle may associate. The second attachment site of the antigen or antigenic determinant may be a protein, a polypeptide, a peptide, a sugar, a polynucleotide, a natural or synthetic polymer, a secondary metabolite or compound (biotin, fluorescein, retinol, digoxigenin, metal ions, phenylmethylsulfonylfluoride), or a combination thereof, or a chemically reactive group thereof. At least one second attachment site is present on the antigen or antigenic determinant. The term “antigen or antigenic determinant with at least one second attachment site” refers, therefore, to an antigen or antigenic construct comprising at least the antigen or antigenic determinant and the second attachment site. However, in particular for a second attachment site, which is of non-natural origin, i.e. not naturally occurring within the antigen or antigenic determinant, these antigen or antigenic constructs comprise an “amino acid linker”.

Bound: As used herein, the term “bound” refers to binding that may be covalent, e.g., by chemically coupling a viral peptide to a virus-like particle, or non-covalent, e.g., ionic interactions, hydrophobic interactions, hydrogen bonds, etc. Covalent bonds can be, for example, ester, ether, phosphoester, amide, peptide, imide, carbon-sulfur bonds, carbon-phosphorus bonds, and the like. The term “bound” is broader than and includes terms such as “coupled,” “fused” and “attached.”

Coat protein(s): As used herein, the term “coat protein(s)” refers to the protein(s) of a bacteriophage or a RNA-phage capable of being incorporated within the capsid assembly of the bacteriophage or the RNA-phage. However, when referring to the specific gene product of the coat protein gene of RNA-phages the term “CP” is used. For example, the specific gene product of the coat protein gene of RNA-phage Qβ is referred to as “Qβ CP”, whereas the “coat proteins” of bacteriophage Qβ comprise the “Qβ CP” as well as the A1 protein. The capsid of Bacteriophage Qβ is composed mainly of the Qβ CP, with a minor content of the A1 protein. Likewise, the VLP Qβ coat protein contains mainly Qβ CP, with a minor content of A1 protein.

Coupled: As used herein, the term “coupled” refers to attachment by covalent bonds or by strong non-covalent interactions. Any method normally used by those skilled in the art for the coupling of biologically active materials can be used in the present invention.

Fusion: As used herein, the term “fusion” refers to the combination of amino acid sequences of different origin in one polypeptide chain by in-frame combination of their coding nucleotide sequences. The term “fusion” explicitly encompasses internal fusions, i.e., insertion of sequences of different origin within a polypeptide chain, in addition to fusion to one of its termini.

CpG: As used herein, the term “CpG” refers to an oligonucleotide which contains an unmethylated cytosine, guanine dinucleotide sequence (e.g. “CpG DNA” or DNA containing a cytosine followed by guanosine and linked by a phosphate bond) and stimulates/activates, e.g. has a mitogenic effect on, or induces and/or increases cytokine expression by, a vertebrate bone marrow derived cell. For example, CpGs can be useful in activating B cells, NK cells and antigen-presenting cells, such as monocytes, dendritic cells and macrophages and T cells. The CpGs can include nucleotide modifications/analogs such as phosphorothioate modifications and can be double-stranded or single-stranded. Generally, double-stranded molecules are more stable in vivo, while single-stranded molecules have increased immune activity.

Epitope: As used herein, the term “epitope” refers to portions of a polypeptide having antigenic or immunogenic activity in an animal, preferably a mammal, and most preferably in a human. An “immunogenic epitope,” as used herein, is defined as a portion of a polypeptide that elicits an antibody response or induces a T-cell response in an animal, as determined by any method known in the art. (See, for example, Geysen et al., Proc. Natl. Acad. Sci. USA 81:3998-4002 (1983)). The term “antigenic epitope,” as used herein, is defined as a portion of a protein to which an antibody can immunospecifically bind its antigen as determined by any method well known in the art Immunospecific binding excludes non-specific binding but does not necessarily exclude cross-reactivity with other antigens. Antigenic epitopes need not necessarily be immunogenic. Antigenic epitopes can also be T-cell epitopes, in which case they can be bound immunospecifically by a T-cell receptor within the context of an MHC molecule.

An epitope can comprise 3 amino acids in a spatial conformation which is unique to the epitope. Generally, an epitope consists of at least about 5 such amino acids, and more usually, consists of at least about 8-10 such amino acids. If the epitope is an organic molecule, it may be as small as Nitrophenyl.

Immune response: As used herein, the term “immune response” refers to a humoral immune response and/or cellular immune response leading to the activation or proliferation of B- and/or T-lymphocytes. In some instances, however, the immune responses may be of low intensity and become detectable only when using at least one substance in accordance with the invention “Immunogenic” refers to an agent used to stimulate the immune system of a living organism, so that one or more functions of the immune system are increased and directed towards the immunogenic agent. An “immunogenic polypeptide” is a polypeptide that elicits a cellular and/or humoral immune response, whether alone or linked to a carrier in the presence or absence of an adjuvant.

Immunization: As used herein, the terms “immunize” or “immunization” or related terms refer to conferring the ability to mount a substantial immune response (comprising antibodies or cellular immunity such as effector CTL) against a target antigen or epitope. These terms do not require that complete immunity be created, but rather that an immune response be produced which is substantially greater than baseline. For example, a mammal may be considered to be immunized against a target antigen if the cellular and/or humoral immune response to the target antigen occurs following the application of methods of the invention.

Immunostimulatory nucleic acid: As used herein, the term immunostimulatory nucleic acid refers to a nucleic acid capable of inducing and/or enhancing an immune response Immunostimulatory nucleic acids, as used herein, comprise ribonucleic acids and in particular deoxyribonucleic acids. Preferably, immunostimulatory nucleic acids contain at least one CpG motif e.g. a CG dinucleotide in which the C is unmethylated. The CG dinucleotide can be part of a palindromic sequence or can be encompassed within a non-palindromic sequence Immunostimulatory nucleic acids not containing CpG motifs as described above encompass, by way of example, nucleic acids lacking CpG dinucleotides, as well as nucleic acids containing CG motifs with a methylated CG dinucleotide. The term “immunostimulatory nucleic acid” as used herein should also refer to nucleic acids that contain modified bases such as 4-bromo-cytosine.

Natural origin: As used herein, the term “natural origin” means that the whole or parts thereof are not synthetic and exist or are produced in nature.

Non-natural: As used herein, the term generally means not from nature, more specifically, the term means from the hand of man.

Non-natural origin: As used herein, the term “non-natural origin” generally means synthetic or not from nature; more specifically, the term means from the hand of man.

Ordered and repetitive antigen or antigenic determinant array: As used herein, the term “ordered and repetitive antigen or antigenic determinant array” generally refers to a repeating pattern of antigen or antigenic determinant, characterized by a typically and preferably uniform spacial arrangement of the antigens or antigenic determinants with respect to the core particle and virus-like particle, respectively. In one embodiment of the invention, the repeating pattern may be a geometric pattern. Typical and preferred examples of suitable ordered and repetitive antigen or antigenic determinant arrays are those which possess strictly repetitive paracrystalline orders of antigens or antigenic determinants, preferably with spacings of 0.5 to 30 nanometers, more preferably 5 to 15 nanometers.

Oligonucleotide: As used herein, the terms “oligonucleotide” or “oligomer” refer to a nucleic acid sequence comprising 2 or more nucleotides, generally at least about 6 nucleotides to about 100,000 nucleotides, preferably about 6 to about 2000 nucleotides, and more preferably about 6 to about 300 nucleotides, even more preferably about 20 to about 300 nucleotides, and even more preferably about 20 to about 100 nucleotides. The terms “oligonucleotide” or “oligomer” also refer to a nucleic acid sequence comprising more than 100 to about 2000 nucleotides, preferably more than 100 to about 1000 nucleotides, and more preferably more than 100 to about 500 nucleotides. “Oligonucleotide” also generally refers to any polyribonucleotide or polydeoxribonucleotide, which may be unmodified RNA or DNA or modified RNA or DNA. “Oligonucleotide” includes, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “oligonucleotide” refers to triple-stranded regions comprising RNA or DNA or both RNA and DNA. Further, an oligonucleotide can be synthetic, genomic or recombinant, e.g., λ-DNA, cosmid DNA, artificial bacterial chromosome, yeast artificial chromosome and filamentous phage such as M13.

The term “oligonucleotide” also includes DNAs or RNAs containing one or more modified bases and DNAs or RNAs with backbones modified for stability or for other reasons. For example, suitable nucleotide modifications/analogs include peptide nucleic acid, inosin, tritylated bases, phosphorothioates, alkylphosphorothioates, 5-nitroindole deoxyribofuranosyl, 5-methyldeoxycytosine and 5,6-dihydro-5,6-dihydroxydeoxythymidine. A variety of modifications have been made to DNA and RNA; thus, “oligonucleotide” embraces chemically, enzymatically and/or metabolically modified forms of polynucleotides as typically found in nature, as well as the chemical forms of DNA and RNA characteristic of viruses and cells. Other nucleotide analogs/modifications will be evident to those skilled in the art.

The compositions of the invention can be combined, optionally, with a pharmaceutically-acceptable carrier. The term “pharmaceutically-acceptable carrier” as used herein means one or more compatible solid or liquid fillers, diluents or encapsulating substances which are suitable for administration into a human or other animal. The term “carrier” denotes an organic or inorganic ingredient, natural or synthetic, with which the active ingredient is combined to facilitate the application.

Organic molecule: As used herein, the term “organic molecule” refers to any chemical entity of natural or synthetic origin. In particular the term “organic molecule” as used herein encompasses, for example, any molecule being a member of the group of nucleotides, lipids, carbohydrates, polysaccharides, lipopolysaccharides, steroids, alkaloids, terpenes and fatty acids, being either of natural or synthetic origin. In particular, the term “organic molecule” encompasses molecules such as nicotine, cocaine, heroin or other pharmacologically active molecules contained in drugs of abuse. In general an organic molecule contains or is modified to contain a chemical functionality allowing its coupling, binding or other method of attachment to the virus-like particle in accordance with the invention.

Polypeptide: As used herein, the term “polypeptide” refers to a molecule composed of monomers (amino acids) linearly linked by amide bonds (also known as peptide bonds). It indicates a molecular chain of amino acids and does not refer to a specific length of the product. Thus, peptides, oligopeptides and proteins are included within the definition of polypeptide. This term is also intended to refer to post-expression modifications of the polypeptide, for example, glycosolations, acetylations, phosphorylations, and the like. A recombinant or derived polypeptide is not necessarily translated from a designated nucleic acid sequence. It may also be generated in any manner, including chemical synthesis.

Substance that activates antigen presenting cells: As used herein, the term “substance that activates antigen presenting cells” refers to a compound which stimulates one or more activities associated with antigen presenting cells. Such activities are well known by those of skill in the art. For example, the substance can stimulate upregulation of costimulatory molecules on antigen presenting cells, induce nuclear translocation of NF-κB in antigen presenting cells, activate toll-like receptors in antigen presenting cells, or other activities involving cytokines or chemokines.

An amount of a substance that activates antigen presenting cells which “enhances” an immune response refers to an amount in which an immune response is observed that is greater or intensified or deviated in any way with the addition of the substance when compared to the same immune response measured without the addition of the substance. For example, the lytic activity of cytotoxic T cells can be measured, e.g. using a ⁵¹Cr release assay, with and without the substance. The amount of the substance at which the CTL lytic activity is enhanced as compared to the CTL lytic activity without the substance is said to be an amount sufficient to enhance the immune response of the animal to the antigen. In a preferred embodiment, the immune response in enhanced by a factor of at least about 2, more preferably by a factor of about 3 or more. The amount of cytokines secreted may also be altered.

Effective Amount: As used herein, the term “effective amount” refers to an amount necessary or sufficient to realize a desired biologic effect. An effective amount of the composition would be the amount that achieves this selected result, and such an amount could be determined as a matter of routine by a person skilled in the art. For example, an effective amount of an oligonucleotide containing at least one unmethylated CpG for treating an immune system deficiency could be that amount necessary to cause activation of the immune system, resulting in the development of an antigen specific immune response upon exposure to antigen. The term is also synonymous with “sufficient amount.”

The effective amount for any particular application can vary depending on such factors as the disease or condition being treated, the particular composition being administered, the size of the subject, and/or the severity of the disease or condition. One of ordinary skill in the art can empirically determine the effective amount of a particular composition of the present invention without necessitating undue experimentation.

Self antigen: As used herein, the tem “self antigen” refers to proteins encoded by the host's DNA and products generated by proteins or RNA encoded by the host's DNA are defined as self. In addition, proteins that result from a combination of two or several self-molecules or that represent a fraction of a self-molecule and proteins that have a high homology two self-molecules as defined above (>95%, preferably >97%, more preferably >99%) may also be considered self. In a further preferred embodiment of the present invention, the antigen is a self antigen. Very preferred embodiments of self-antigens useful for the present invention are described in WO 02/056905, the disclosure of which is herewith incorporated by reference in its entirety.

Treatment: As used herein, the terms “treatment”, “treat”, “treated”, or “treating” refer to prophylaxis and/or therapy. When used with respect to an infectious disease, for example, the term refers to a prophylactic treatment which increases the resistance of a subject to infection with a pathogen or, in other words, decreases the likelihood that the subject will become infected with the pathogen or will show signs of illness attributable to the infection, as well as a treatment after the subject has become infected in order to fight the infection, e.g., reduce or eliminate the infection or prevent it from becoming worse.

Vaccine: As used herein, the term “vaccine” refers to a formulation which contains the composition of the present invention and which is in a form that is capable of being administered to an animal. Typically, the vaccine comprises a conventional saline or buffered aqueous solution medium in which the composition of the present invention is suspended or dissolved. In this form, the composition of the present invention can be used conveniently to prevent, ameliorate, or otherwise treat a condition. Upon introduction into a host, the vaccine is able to provoke an immune response including, but not limited to, the production of antibodies, cytokines and/or other cellular responses.

Optionally, the vaccine of the present invention additionally includes an adjuvant which can be present in either a minor or major proportion relative to the compound of the present invention. The term “adjuvant” as used herein refers to non-specific stimulators of the immune response or substances that allow generation of a depot in the host which when combined with the vaccine of the present invention provide for an even more enhanced immune response. A variety of adjuvants can be used. Examples include incomplete Freund's adjuvant, aluminum hydroxide and modified muramyldipeptide. The term “adjuvant” as used herein also refers to typically specific stimulators of the immune response which when combined with the vaccine of the present invention provide for an even more enhanced and typically specific immune response. Examples include, but limited to, GM-CSF, IL-2, IL-12, IFNα. Further examples are within the knowledge of the person skilled in the art.

Virus-like particle: As used herein, the term “virus-like particle” refers to a structure resembling a virus particle but which has not been demonstrated to be pathogenic. Typically, a virus-like particle in accordance with the invention does not carry genetic information encoding for the proteins of the virus-like particle. In general, virus-like particles lack the viral genome and, therefore, are noninfectious. Also, virus-like particles can often be produced in large quantities by heterologous expression and can be easily purified. Some virus-like particles may contain nucleic acid distinct from their genome. As indicated, a virus-like particle in accordance with the invention is non replicative and noninfectious since it lacks all or part of the viral genome, in particular the replicative and infectious components of the viral genome. A virus-like particle in accordance with the invention may contain nucleic acid distinct from their genome. A typical and preferred embodiment of a virus-like particle in accordance with the present invention is a viral capsid such as the viral capsid of the corresponding virus, bacteriophage, or RNA-phage. The terms “viral capsid” or “capsid”, as interchangeably used herein, refer to a macromolecular assembly composed of viral protein subunits. Typically and preferably, the viral protein subunits assemble into a viral capsid and capsid, respectively, having a structure with an inherent repetitive organization, wherein said structure is, typically, spherical or tubular. For example, the capsids of RNA-phages or HBcAg's have a spherical form of icosahedral symmetry. The term “capsid-like structure” as used herein, refers to a macromolecular assembly composed of viral protein subunits reassembling the capsid morphology in the above defined sense but deviating from the typical symmetrical assembly while maintaining a sufficient degree of order and repetitiveness.

Virus-like particle of a bacteriophage: As used herein, the term “virus-like particle of a bacteriophage” refers to a virus-like particle resembling the structure of a bacteriophage, being non replicative and noninfectious, and lacking at least the gene or genes encoding for the replication machinery of the bacteriophage, and typically also lacking the gene or genes encoding the protein or proteins responsible for viral attachment to or entry into the host. This definition should, however, also encompass virus-like particles of bacteriophages, in which the aforementioned gene or genes are still present but inactive, and, therefore, also leading to non-replicative and noninfectious virus-like particles of a bacteriophage.

VLP of RNA phage coat protein: The capsid structure formed from the self-assembly of 180 subunits of RNA phage coat protein and optionally containing host RNA is referred to as a “VLP of RNA phage coat protein”. A specific example is the VLP of Qβ coat protein. In this particular case, the VLP of Qβ coat protein may either be assembled exclusively from Qβ CP subunits (generated by expression of a Qβ CP gene containing, for example, a TAA stop codon precluding any expression of the longer A1 protein through suppression, see Kozlovska, T. M., et al., Intervirology 39: 9-15 (1996)), or additionally contain A1 protein subunits in the capsid assembly.

The term “virus particle” as used herein refers to the morphological form of a virus. In some virus types it comprises a genome surrounded by a protein capsid; others have additional structures (e.g., envelopes, tails, etc.).

Non-enveloped viral particles are made up of a proteinaceous capsid that surrounds and protects the viral genome. Enveloped viruses also have a capsid structure surrounding the genetic material of the virus but, in addition, have a lipid bilayer envelope that surrounds the capsid. In one embodiment of the invention, the virus-like particles are free of a lipoprotein envelope or a lipoprotein-containing envelope. In a further embodiment, the virus-like particles are free of an envelope altogether.

One, a or an: When the terms “one,” “a,” or “an” are used in this disclosure, they mean “at least one” or “one or more,” unless otherwise indicated.

As will be clear to those skilled in the art, certain embodiments of the invention involve the use of recombinant nucleic acid technologies such as cloning, polymerase chain reaction, the purification of DNA and RNA, the expression of recombinant proteins in prokaryotic and eukaryotic cells, etc. Such methodologies are well known to those skilled in the art and can be conveniently found in published laboratory methods manuals (e.g., Sambrook, J. et al., eds., MOLECULAR CLONING, A LABORATORY MANUAL, 2nd. edition, Cold Spring Harbor Laboratory Press, Cold Spring Harbor, N.Y. (1989); Ausubel, F. et al., eds., CURRENT PROTOCOLS IN MOLECULAR BIOLOGY, John H. Wiley & Sons, Inc. (1997)). Fundamental laboratory techniques for working with tissue culture cell lines (Celis, J., ed., CELL BIOLOGY, Academic Press, 2^{nd} edition, (1998)) and antibody-based technologies (Harlow, E. and Lane, D., “Antibodies: A Laboratory Manual,” Cold Spring Harbor Laboratory, Cold Spring Harbor, N.Y. (1988); Deutscher, M. P., “Guide to Protein Purification,” Meth. Enzymol. 128, Academic Press San Diego (1990); Scopes, R. K., “Protein Purification Principles and Practice,” 3^{rd} ed., Springer-Verlag, New York (1994)) are also adequately described in the literature, all of which are incorporated herein by reference.

### 2. Compositions and Methods for Enhancing an Immune Response

The disclosed invention provides compositions and methods for enhancing an immune response against an antigen in an animal. Compositions of the invention comprise, or alternatively consist of, a virus-like particle coupled, fused or otherwise attached to an antigen capable of inducing an immune response against the antigen in the animal and a substance that activates antigen presenting cells in an amount sufficient to enhance the immune response of the animal to the antigen. Furthermore, the invention conveniently enables the practitioner to construct such a composition for various treatment and/or prophylactic prevention purposes, which include the prevention and/or treatment of infectious diseases, as well as chronic infectious diseases, and the prevention and/or treatment of cancers, for example.

Virus-like particles in the context of the present application refer to structures resembling a virus particle but which are not pathogenic. In general, virus-like particles lack the viral genome and, therefore, are noninfectious. Also, virus-like particles can be produced in large quantities by heterologous expression and can be easily purified.

In a preferred embodiment, the virus-like particle is a recombinant virus-like particle. The skilled artisan can produce VLPs using recombinant DNA technology and virus coding sequences which are readily available to the public. For example, the coding sequence of a virus envelope or core protein can be engineered for expression in a baculovirus expression vector using a commercially available baculovirus vector, under the regulatory control of a virus promoter, with appropriate modifications of the sequence to allow functional linkage of the coding sequence to the regulatory sequence. The coding sequence of a virus envelope or core protein can also be engineered for expression in a bacterial expression vector, for example.

Examples of VLPs include, but are not limited to, the capsid proteins of Hepatitis B virus (Ulrich, et al., Virus Res. 50:141-182 (1998)), measles virus (Warnes, et al., Gene 160:173-178 (1995)), Sindbis virus, rotavirus (U.S. Pat. Nos. 5,071,651 and 5,374,426), foot-and-mouth-disease virus (Twomey, et al., Vaccine 13:1603-1610, (1995)), Norwalk virus (Jiang, X., et al., Science 250:1580-1583 (1990); Matsui, S. M., et al., J. Clin. Invest. 87:1456-1461 (1991)), the retroviral GAG protein (PCT Patent Appl. No. WO 96/30523), the retrotransposon Ty protein p1, the surface protein of Hepatitis B virus (WO 92/11291), human papilloma virus (WO 98/15631), RNA phages, fr-phage, GA-phage, AP 205-phage, Ty and, in particular, Qβ-phage.

As will be readily apparent to those skilled in the art, the VLP of the invention is not limited to any specific form. The particle can be synthesized chemically or through a biological process, which can be natural or non-natural. By way of example, this type of embodiment includes a virus-like particle or a recombinant form thereof. In a more specific embodiment, the VLP can comprise, or alternatively consist of, recombinant polypeptides of Rotavirus, recombinant polypeptides of Norwalk virus, recombinant polypeptides of Alphavirus, recombinant proteins which form bacterial pili or pilus-like structures, recombinant polypeptides of Foot and Mouth Disease virus, recombinant polypeptides of measles virus, recombinant polypeptides of Sindbis virus, recombinant polypeptides of Retrovirus; recombinant polypeptides of Hepatitis B virus (e.g., a HBcAg); recombinant polypeptides of Tobacco mosaic virus; recombinant polypeptides of Flock House Virus; recombinant polypeptides of human Papillomavirus; recombinant polypeptides of Polyoma virus and, in particular, recombinant polypeptides of human Polyoma virus, and in particular recombinant polypeptides of BK virus; recombinant polypeptides of bacteriophages, recombinant polypeptides of RNA phages; recombinant polypeptides of Ty; recombinant polypeptides of fr-phage, recombinant polypeptides of GA-phage, recombinant polypeptides of AP 205-phage and, in particular, recombinant polypeptides of Qβ-phage. The virus-like particle can further comprise, or alternatively consist of, one or more fragments of such polypeptides, as well as variants of such polypeptides. Variants of polypeptides can share, for example, at least 80%, 85%, 90%, 95%, 97%, or 99% identity at the amino acid level with their wild-type counterparts.

In a preferred embodiment, the virus-like particle comprises, consists essentially of, or alternatively consists of recombinant proteins, or fragments thereof, of a RNA-phage. Preferably, the RNA-phage is selected from the group consisting of a) bacteriophage Qβ; b) bacteriophage R17; c) bacteriophage fr; d) bacteriophage GA; e) bacteriophage SP; f) bacteriophage MS2; g) bacteriophage M11; h) bacteriophage MX1; i) bacteriophage NL95; k) bacteriophage f2; and l) bacteriophage PP7 and bacteriophage AP205.

In another preferred embodiment of the present invention, the virus-like particle comprises, or alternatively consists essentially of, or alternatively consists of recombinant proteins, or fragments thereof, of the RNA-bacteriophage Qβ or of the RNA-bacteriophage fr.

In a further preferred embodiment of the present invention, the recombinant proteins comprise, or alternatively consist essentially of, or alternatively consist of coat proteins of RNA phages.

RNA-phage coat proteins forming capsids or VLPs, or fragments of the bacteriophage coat proteins compatible with self-assembly into a capsid or a VLP, are, therefore, further preferred embodiments of the present invention. Bacteriophage Qβ coat proteins, for example, can be expressed recombinantly in E. coli. Further, upon such expression these proteins spontaneously form capsids. Additionally, these capsids form a structure with an inherent repetitive organization.

Specific preferred examples of bacteriophage coat proteins which can be used to prepare compositions of the invention include the coat proteins of RNA bacteriophages such as bacteriophage Qβ (SEQ ID NO:10; PIR Database, Accession No. VCBPQβ referring to Qβ CP and SEQ ID NO: 11; Accession No. AAA16663 referring to Qβ A1 protein), bacteriophage R17 (SEQ ID NO:12; PIR Accession No. VCBPR7), bacteriophage fr (SEQ ID NO:13; PIR Accession No. VCBPFR), bacteriophage GA (SEQ ID NO:14; GenBank Accession No. NP-040754), bacteriophage SP (SEQ ID NO:15; GenBank Accession No. CAA30374 referring to SP CP and SEQ ID NO: 16; Accession No. referring to SP A1 protein), bacteriophage MS2 (SEQ ID NO:17; PIR Accession No. VCBPM2), bacteriophage M11 (SEQ ID NO:18; GenBank Accession No. AAC06250), bacteriophage MX1 (SEQ ID NO:19; GenBank Accession No. AAC14699), bacteriophage NL95 (SEQ ID NO:20; GenBank Accession No. AAC14704), bacteriophage f2 (SEQ ID NO: 21; GenBank Accession No. P03611), bacteriophage PP7 (SEQ ID NO: 22). Furthermore, the A1 protein of bacteriophage Qβ or C-terminal truncated forms missing as much as 100, 150 or 180 amino acids from its C-terminus may be incorporated in a capsid assembly of Qβ coat proteins. Generally, the percentage of Qβ A1 protein relative to Qβ CP in the capsid assembly will be limited, in order to ensure capsid formation.

Qβ coat protein has also been found to self-assemble into capsids when expressed in E. coli (Kozlovska T M. et al., GENE 137: 133-137 (1993)). The obtained capsids or virus-like particles showed an icosahedral phage-like capsid structure with a diameter of 25 nm and T=3 quasi symmetry. Further, the crystal structure of phage Qβ has been solved. The capsid contains 180 copies of the coat protein, which are linked in covalent pentamers and hexamers by disulfide bridges (Golmohammadi, R. et al., Structure 4: 543-5554 (1996)) leading to a remarkable stability of the capsid of Qβ coat protein. Capsids or VLPs made from recombinant Qβ coat protein may contain, however, subunits not linked via disulfide links to other subunits within the capsid, or incompletely linked. Thus, upon loading recombinant Qβ capsid on non-reducing SDS-PAGE, bands corresponding to monomeric Qβ coat protein as well as bands corresponding to the hexamer or pentamer of Qβ coat protein are visible. Incompletely disulfide-linked subunits could appear as dimer, trimer or even tetramer bands in non-reducing SDS-PAGE. Qβ capsid protein also shows unusual resistance to organic solvents and denaturing agents. Surprisingly, we have observed that DMSO and acetonitrile concentrations as high as 30%, and Guanidinium concentrations as high as 1 M do not affect the stability of the capsid. The high stability of the capsid of Qβ coat protein is an advantageous feature, in particular, for its use in immunization and vaccination of mammals and humans in accordance of the present invention.

Upon expression in E. coli, the N-terminal methionine of Qβ coat protein is usually removed, as we observed by N-terminal Edman sequencing as described in Stoll, E. et al., J. Biol. Chem. 252:990-993 (1977). VLP composed from Qβ coat proteins where the N-terminal methionine has not been removed, or VLPs comprising a mixture of Qβ coat proteins where the N-terminal methionine is either cleaved or present are also within the scope of the present invention.

Further RNA phage coat proteins have also been shown to self-assemble upon expression in a bacterial host (Kastelein, R A. et al., Gene 23: 245-254 (1983), Kozlovskaya, T M. et al., Dokl. Akad. Nauk SSSR 287: 452-455 (1986), Adhin, M R. et al., Virology 170: 238-242 (1989), Ni, C Z., et al., Protein Sci. 5: 2485-2493 (1996), Priano, C. et al., J. Mol. Biol. 249: 283-297 (1995)). The Qβ phage capsid contains, in addition to the coat protein, the so called read-through protein A1 and the maturation protein A2. A1 is generated by suppression at the UGA stop codon and has a length of 329 aa. The capsid of phage Qβ recombinant coat protein used in the invention is devoid of the A2 lysis protein, and contains RNA from the host. The coat protein of RNA phages is an RNA binding protein, and interacts with the stem loop of the ribosomal binding site of the replicase gene acting as a translational repressor during the life cycle of the virus. The sequence and structural elements of the interaction are known (Witherell, G W. & Uhlenbeck, O C. Biochemistry 28: 71-76 (1989); Lim F. et al., J. Biol. Chem. 271: 31839-31845 (1996)). The stem loop and RNA in general are known to be involved in the virus assembly (Golmohammadi, R. et al., Structure 4: 543-5554 (1996)).

In a further preferred embodiment of the present invention, the virus-like particle comprises, or alternatively consists essentially of, or alternatively consists of recombinant proteins, or fragments thereof, of a RNA-phage, wherein the recombinant proteins comprise, consist essentially of or alternatively consist of mutant coat proteins of a RNA phage, preferably of mutant coat proteins of the RNA phages mentioned above. In another preferred embodiment, the mutant coat proteins of the RNA phage have been modified by removal of at least one lysine residue by way of substitution, or by addition of at least one lysine residue by way of substitution; alternatively, the mutant coat proteins of the RNA phage have been modified by deletion of at least one lysine residue, or by addition of at least one lysine residue by way of insertion.

In another preferred embodiment, the virus-like particle comprises, or alternatively consists essentially of, or alternatively consists of recombinant proteins, or fragments thereof, of the RNA-bacteriophage Qβ, wherein the recombinant proteins comprise, or alternatively consist essentially of, or alternatively consist of coat proteins having an amino acid sequence of SEQ ID NO:10, or a mixture of coat proteins having amino acid sequences of SEQ ID NO:10 and of SEQ ID NO: 11 or mutants of SEQ ID NO: 11 and wherein the N-terminal methionine is preferably cleaved.

In a further preferred embodiment of the present invention, the virus-like particle comprises, consists essentially of or alternatively consists of recombinant proteins of Qβ, or fragments thereof, wherein the recombinant proteins comprise, or alternatively consist essentially of, or alternatively consist of mutant Qβ coat proteins. In another preferred embodiment, these mutant coat proteins have been modified by removal of at least one lysine residue by way of substitution, or by addition of at least one lysine residue by way of substitution. Alternatively, these mutant coat proteins have been modified by deletion of at least one lysine residue, or by addition of at least one lysine residue by way of insertion.

Four lysine residues are exposed on the surface of the capsid of Qβ coat protein. Qβ mutants, for which exposed lysine residues are replaced by arginines can also be used for the present invention. The following Qβ coat protein mutants and mutant Qβ VLPs can, thus, be used in the practice of the invention: “Qβ-240” (Lys13-Arg; SEQ ID NO:23), “Qβ-243” (Asn 10-Lys; SEQ ID NO:24), “Qβ-250” (Lys 2-Arg, Lys13-Arg; SEQ ID NO:25), “Qβ-251” (SEQ ID NO:26) and “Qβ-259” (Lys 2-Arg, Lys16-Arg; SEQ ID NO:27). Thus, in further preferred embodiment of the present invention, the virus-like particle comprises, consists essentially of or alternatively consists of recombinant proteins of mutant Qβ coat proteins, which comprise proteins having an amino acid sequence selected from the group of a) the amino acid sequence of SEQ ID NO: 23; b) the amino acid sequence of SEQ ID NO:24; c) the amino acid sequence of SEQ ID NO: 25; d) the amino acid sequence of SEQ ID NO:26; and e) the amino acid sequence of SEQ ID NO: 27. The construction, expression and purification of the above indicated Qβ coat proteins, mutant Qβ coat protein VLPs and capsids, respectively, are disclosed in pending U.S. application Ser. No. 10/050,902 filed on Jan. 18, 2002. In particular is hereby referred to Example 18 of above mentioned application.

In a further preferred embodiment of the present invention, the virus-like particle comprises, or alternatively consists essentially of, or alternatively consists of recombinant proteins of Qβ, or fragments thereof, wherein the recombinant proteins comprise, consist essentially of or alternatively consist of a mixture of either one of the foregoing Qβ mutants and the corresponding A1 protein.

In a further preferred embodiment of the present invention, the virus-like particle comprises, or alternatively essentially consists of, or alternatively consists of recombinant proteins, or fragments thereof, of RNA-phage AP205.

The AP205 genome consists of a maturation protein, a coat protein, a replicase and two open reading frames not present in related phages; a lysis gene and an open reading frame playing a role in the translation of the maturation gene (Klovins, J., et al., J. Gen. Virol. 83: 1523-33 (2002)). AP205 coat protein can be expressed from plasmid pAP283-58 (SEQ ID NO: 79), which is a derivative of pQb10 (Kozlovska, T. M. et al., Gene 137:133-37 (1993)), and which contains an AP205 ribosomal binding site. Alternatively, AP205 coat protein may be cloned into pQb185, downstream of the ribosomal binding site present in the vector. Both approaches lead to expression of the protein and formation of capsids as described in the co-pending US provisional patent application with the title “Molecular Antigen Arrays” (Application No. 60/396,126) and having been filed on Jul. 17, 2002, which is incorporated by reference in its entirety. Vectors pQb10 and pQb185 are vectors derived from pGEM vector, and expression of the cloned genes in these vectors is controlled by the trp promoter (Kozlovska, T. M. et al., Gene 137:133-37 (1993)). Plasmid pAP283-58 (SEQ ID NO:79) comprises a putative AP205 ribosomal binding site in the following sequence, which is downstream of the XbaI site, and immediately upstream of the ATG start codon of the AP205 coat protein: tctagaATTTTCTGCGCACCCAT CCCGGGTGGCGCCCAAAGTGAGGAAAATCACatg. The vector pQb185 comprises a Shine Delagarno sequence downstream from the XbaI site and upstream of the start codon (tctagaTTAACCCAACGCGTAGGAGTCAGGCCatg, Shine Delagarno sequence underlined).

In a further preferred embodiment of the present invention, the virus-like particle comprises, or alternatively essentially consists of, or alternatively consists of recombinant coat proteins, or fragments thereof, of the RNA-phage AP205.

This preferred embodiment of the present invention, thus, comprises AP205 coat proteins that form capsids. Such proteins are recombinantly expressed, or prepared from natural sources. AP205 coat proteins produced in bacteria spontaneously form capsids, as evidenced by Electron Microscopy (EM) and immunodiffusion. The structural properties of the capsid formed by the AP205 coat protein (SEQ ID NO: 80) and those formed by the coat protein of the AP205 RNA phage are nearly indistinguishable when seen in EM. AP205 VLPs are highly immunogenic, and can be linked with antigens and/or antigenic determinants to generate vaccine constructs displaying the antigens and/or antigenic determinants oriented in a repetitive manner. High titers are elicited against the so displayed antigens showing that bound antigens and/or antigenic determinants are accessible for interacting with antibody molecules and are immunogenic.

In a further preferred embodiment of the present invention, the virus-like particle comprises, or alternatively essentially consists of, or alternatively consists of recombinant mutant coat proteins, or fragments thereof, of the RNA-phage AP205.

Assembly-competent mutant forms of AP205 VLPs, including AP205 coat protein with the substitution of proline at amino acid 5 to threonine (SEQ ID NO: 81), may also be used in the practice of the invention and leads to a further preferred embodiment of the invention. These VLPs, AP205 VLPs derived from natural sources, or AP205 viral particles, may be bound to antigens to produce ordered repetitive arrays of the antigens in accordance with the present invention.

AP205 P5-T mutant coat protein can be expressed from plasmid pAP281-32 (SEQ ID No. 82), which is derived directly from pQb185, and which contains the mutant AP205 coat protein gene instead of the Qβ coat protein gene. Vectors for expression of the AP205 coat protein are transfected into E. coli for expression of the AP205 coat protein.

Methods for expression of the coat protein and the mutant coat protein, respectively, leading to self-assembly into VLPs are described in co-pending US provisional patent application with the title “Molecular Antigen Arrays” (Application No. 60/396,126) and having been filed on Jul. 17, 2002, which is incorporated by reference in its entirety. Suitable E. coli strains include, but are not limited to, E. coli K802, JM 109, RR1. Suitable vectors and strains and combinations thereof can be identified by testing expression of the coat protein and mutant coat protein, respectively, by SDS-PAGE and capsid formation and assembly by optionally first purifying the capsids by gel filtration and subsequently testing them in an immunodiffusion assay (Ouchterlony test) or Electron Microscopy (Kozlovska, T. M. et al., Gene 137:133-37 (1993)).

AP205 coat proteins expressed from the vectors pAP283-58 and pAP281-32 may be devoid of the initial Methionine amino-acid, due to processing in the cytoplasm of E. coli. Cleaved, uncleaved forms of AP205 VLP, or mixtures thereof are further preferred embodiments of the invention.

In a further preferred embodiment of the present invention, the virus-like particle comprises, or alternatively essentially consists of, or alternatively consists of a mixture of recombinant coat proteins, or fragments thereof, of the RNA-phage AP205 and of recombinant mutant coat proteins, or fragments thereof, of the RNA-phage AP205.

In a further preferred embodiment of the present invention, the virus-like particle comprises, or alternatively essentially consists of, or alternatively consists of fragments of recombinant coat proteins or recombinant mutant coat proteins of the RNA-phage AP205.

Recombinant AP205 coat protein fragments capable of assembling into a VLP and a capsid, respectively are also useful in the practice of the invention. These fragments may be generated by deletion, either internally or at the termini of the coat protein and mutant coat protein, respectively. Insertions in the coat protein and mutant coat protein sequence or fusions of antigen sequences to the coat protein and mutant coat protein sequence, and compatible with assembly into a VLP, are further embodiments of the invention and lead to chimeric AP205 coat proteins, and particles, respectively. The outcome of insertions, deletions and fusions to the coat protein sequence and whether it is compatible with assembly into a VLP can be determined by electron microscopy.

The particles formed by the AP205 coat protein, coat protein fragments and chimeric coat proteins described above, can be isolated in pure form by a combination of fractionation steps by precipitation and of purification steps by gel filtration using e.g. Sepharose CL-4B, Sepharose CL-2B, Sepharose CL-6B columns and combinations thereof as described in the co-pending US provisional patent application with the title “Molecular Antigen Arrays” (Application No. 60/396,126) and having been filed on Jul. 17, 2002, which is incorporated by reference in its entirety. Other methods of isolating virus-like particles are known in the art, and may be used to isolate the virus-like particles (VLPs) of bacteriophage AP205. For example, the use of ultracentrifugation to isolate VLPs of the yeast retrotransposon Ty is described in U.S. Pat. No. 4,918,166, which is incorporated by reference herein in its entirety.

The crystal structure of several RNA bacteriophages has been determined (Golmohammadi, R. et al., Structure 4:543-554 (1996)). Using such information, surface exposed residues can be identified and, thus, RNA-phage coat proteins can be modified such that one or more reactive amino acid residues can be inserted by way of insertion or substitution. As a consequence, those modified forms of bacteriophage coat proteins can also be used for the present invention. Thus, variants of proteins which form capsids or capsid-like structures (e.g., coat proteins of bacteriophage Qβ, bacteriophage R17, bacteriophage fr, bacteriophage GA, bacteriophage SP, and bacteriophage MS2) can also be used to prepare compositions of the present invention.

Although the sequence of the variants proteins discussed above will differ from their wild-type counterparts, these variant proteins will generally retain the ability to form capsids or capsid-like structures. Thus, the invention further includes compositions and vaccine compositions, respectively, which further includes variants of proteins which form capsids or capsid-like structures, as well as methods for preparing such compositions and vaccine compositions, respectively, individual protein subunits used to prepare such compositions, and nucleic acid molecules which encode these protein subunits. Thus, included within the scope of the invention are variant forms of wild-type proteins which form capsids or capsid-like structures and retain the ability to associate and form capsids or capsid-like structures.

As a result, the invention further includes compositions and vaccine compositions, respectively, comprising proteins, which comprise, or alternatively consist essentially of, or alternatively consist of amino acid sequences which are at least 80%, 85%, 90%, 95%, 97%, or 99% identical to wild-type proteins which form ordered arrays and have an inherent repetitive structure, respectively.

Further included within the scope of the invention are nucleic acid molecules which encode proteins used to prepare compositions of the present invention.

In other embodiments, the invention further includes compositions comprising proteins, which comprise, or alternatively consist essentially of, or alternatively consist of amino acid sequences which are at least 80%, 85%, 90%, 95%, 97%, or 99% identical to any of the amino acid sequences shown in SEQ ID NOs:10-27.

Proteins suitable for use in the present invention also include C-terminal truncation mutants of proteins which form capsids or capsid-like structures, or VLPs. Specific examples of such truncation mutants include proteins having an amino acid sequence shown in any of SEQ ID NOs:10-27 where 1, 2, 5, 7, 9, 10, 12, 14, 15, or 17 amino acids have been removed from the C-terminus Typically, theses C-terminal truncation mutants will retain the ability to form capsids or capsid-like structures.

Further proteins suitable for use in the present invention also include N-terminal truncation mutants of proteins which form capsids or capsid-like structures. Specific examples of such truncation mutants include proteins having an amino acid sequence shown in any of SEQ ID NOs:10-27 where 1, 2, 5, 7, 9, 10, 12, 14, 15, or 17 amino acids have been removed from the N-terminus Typically, these N-terminal truncation mutants will retain the ability to form capsids or capsid-like structures.

Additional proteins suitable for use in the present invention include N- and C-terminal truncation mutants which form capsids or capsid-like structures. Suitable truncation mutants include proteins having an amino acid sequence shown in any of SEQ ID NOs:10-27 where 1, 2, 5, 7, 9, 10, 12, 14, 15, or 17 amino acids have been removed from the N-terminus and 1, 2, 5, 7, 9, 10, 12, 14, 15, or 17 amino acids have been removed from the C-terminus. Typically, these N-terminal and C-terminal truncation mutants will retain the ability to form capsids or capsid-like structures.

The invention further includes compositions comprising proteins which comprise, or alternatively consist essentially of, or alternatively consist of, amino acid sequences which are at least 80%, 85%, 90%, 95%, 97%, or 99% identical to the above described truncation mutants.

The invention thus includes compositions and vaccine compositions prepared from proteins which form capsids or VLPs, methods for preparing these compositions from individual protein subunits and VLPs or capsids, methods for preparing these individual protein subunits, nucleic acid molecules which encode these subunits, and methods for vaccinating and/or eliciting immunological responses in individuals using these compositions of the present invention.

Fragments of VLPs which retain the ability to induce an immune response can comprise, or alternatively consist of, polypeptides which are about 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450 or 500 amino acids in length, but will obviously depend on the length of the sequence of the subunit composing the VLP. Examples of such fragments include fragments of proteins discussed herein which are suitable for the preparation of the immune response enhancing composition.

In another preferred embodiment of the invention, the VLP's are free of a lipoprotein envelope or a lipoprotein-containing envelope. In a further preferred embodiment, the VLP's are free of an envelope altogether.

The lack of a lipoprotein envelope or lipoprotein-containing envelope and, in particular, the complete lack of an envelope leads to a more defined virus-like particle in its structure and composition. Such more defined virus-like particles, therefore, may minimize side-effects. Moreover, the lack of a lipoprotein-containing envelope or, in particular, the complete lack of an envelope avoids or minimizes incorporation of potentially toxic molecules and pyrogens within the virus-like particle.

As previously stated, the invention includes virus-like particles or recombinant forms thereof. Skilled artisans have the knowledge to produce such particles and attach antigens thereto. By way of providing other examples, the invention provides herein for the production of Hepatitis B virus-like particles as virus-like particles (Example 1).

Antigens fused to the virus-like particle by insertion within the sequence of the virus-like particle building monomer is also within the scope of the present invention. In some cases, antigens may be inserted in a form of the virus-like particle building monomer containing deletions. In these cases, the virus-like particle building monomer may not be able to form virus-like structures in the absence of the inserted antigen.

In one embodiment, the particles used in compositions of the invention are composed of a Hepatitis B capsid (core) protein (HBcAg) or a fragment of a HBcAg which has been modified to either eliminate or reduce the number of free cysteine residues. Zhou et al. (J. Virol. 66:5393-5398 (1992)) demonstrated that HBcAgs which have been modified to remove the naturally resident cysteine residues retain the ability to associate and form multimeric structures. Thus, core particles suitable for use in compositions of the invention include those comprising modified HBcAgs, or fragments thereof, in which one or more of the naturally resident cysteine residues have been either deleted or substituted with another amino acid residue (e.g., a serine residue).

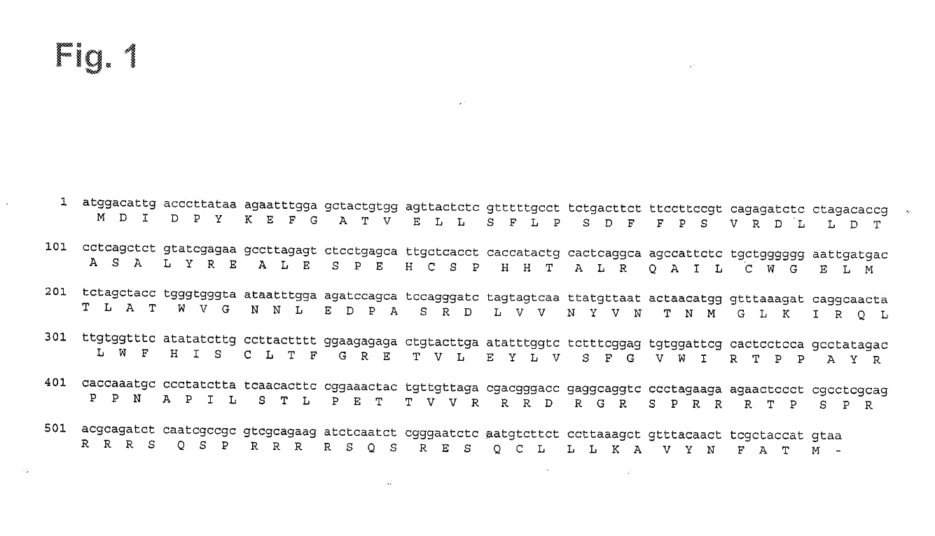

The HBcAg is a protein generated by the processing of a Hepatitis B core antigen precursor protein. A number of isotypes of the HBcAg have been identified and their amino acids sequences are readily available to those skilled in the art. For example, the HBcAg protein having the amino acid sequence shown in FIG. 1 is 183 amino acids in length and is generated by the processing of a 212 amino acid Hepatitis B core antigen precursor protein. This processing results in the removal of 29 amino acids from the N-terminus of the Hepatitis B core antigen precursor protein. Similarly, the HBcAg protein that is 185 amino acids in length is generated by the processing of a 214 amino acid Hepatitis B core antigen precursor protein.

In preferred embodiments, vaccine compositions of the invention will be prepared using the processed form of a HBcAg (i.e., a HBcAg from which the N-terminal leader sequence of the Hepatitis B core antigen precursor protein have been removed).

Further, when HBcAgs are produced under conditions where processing will not occur, the HBcAgs will generally be expressed in “processed” form. For example, bacterial systems, such as E. coli, generally do not remove the leader sequences, also referred to as “signal peptides,” of proteins which are normally expressed in eukaryotic cells. Thus, when an E. coli expression system directing expression of the protein to the cytoplasm is used to produce HBcAgs of the invention, these proteins will generally be expressed such that the N-terminal leader sequence of the Hepatitis B core antigen precursor protein is not present.

The preparation of Hepatitis B virus-like particles, which can be used for the present invention, is disclosed, for example, in WO 00/32227, and hereby in particular in Examples 17 to 19 and 21 to 24, as well as in WO 01/85208, and hereby in particular in Examples 17 to 19, 21 to 24, 31 and 41, and in pending U.S. application Ser. No. 10/050,902 filed on Jan. 18, 2002. For the latter application, it is in particular referred to Example 23, 24, 31 and 51. All three documents are explicitly incorporated herein by reference.

The present invention also includes HBcAg variants which have been modified to delete or substitute one or more additional cysteine residues. Thus, the vaccine compositions of the invention include compositions comprising HBcAgs in which cysteine residues not present in the amino acid sequence shown in FIG. 1 have been deleted.

It is well known in the art that free cysteine residues can be involved in a number of chemical side reactions. These side reactions include disulfide exchanges, reaction with chemical substances or metabolites that are, for example, injected or formed in a combination therapy with other substances, or direct oxidation and reaction with nucleotides upon exposure to UV light. Toxic adducts could thus be generated, especially considering the fact that HBcAgs have a strong tendency to bind nucleic acids. The toxic adducts would thus be distributed between a multiplicity of species, which individually may each be present at low concentration, but reach toxic levels when together.

In view of the above, one advantage to the use of HBcAgs in vaccine compositions which have been modified to remove naturally resident cysteine residues is that sites to which toxic species can bind when antigens or antigenic determinants are attached would be reduced in number or eliminated altogether.

A number of naturally occurring HBcAg variants suitable for use in the practice of the present invention have been identified. Yuan et al., (J. Virol. 73:10122-10128 (1999)), for example, describe variants in which the isoleucine residue at position corresponding to position 97 in SEQ ID NO:28 is replaced with either a leucine residue or a phenylalanine residue. The amino acid sequences of a number of HBcAg variants, as well as several Hepatitis B core antigen precursor variants, are disclosed in GenBank reports AAF121240 (SEQ ID NO:29), AF121239 (SEQ ID NO:30), X85297 (SEQ ID NO:31), X02496 (SEQ ID NO:32), X85305 (SEQ ID NO:33), X85303 (SEQ ID NO:34), AF151735 (SEQ ID NO:35), X85259 (SEQ ID NO:36), X85286 (SEQ ID NO:37), X85260 (SEQ ID NO:38), X85317 (SEQ ID NO:39), X85298 (SEQ ID NO:40), AF043593 (SEQ ID NO:41), M20706 (SEQ ID NO:42), X85295 (SEQ ID NO:43), X80925 (SEQ ID NO:44), X85284 (SEQ ID NO:45), X85275 (SEQ ID NO:46), X72702 (SEQ ID NO:47), X85291 (SEQ ID NO:48), X65258 (SEQ ID NO:49), X85302 (SEQ ID NO:50), M32138 (SEQ ID NO:51), X85293 (SEQ ID NO:52), X85315 (SEQ ID NO:53), U95551 (SEQ ID NO:54), X85256 (SEQ ID NO:55), X85316 (SEQ ID NO:56), X85296 (SEQ ID NO:57), AB033559 (SEQ ID NO:58), X59795 (SEQ ID NO:59), X85299 (SEQ ID NO:60), X85307 (SEQ ID NO:61), X65257 (SEQ ID NO:62), X85311 (SEQ ID NO:63), X85301 (SEQ ID NO:64), X85314 (SEQ ID NO:65), X85287 (SEQ ID NO:66), X85272 (SEQ ID NO:67), X85319 (SEQ ID NO:68), AB010289 (SEQ ID NO:69), X85285 (SEQ ID NO:70), AB010289 (SEQ ID NO:71), AF121242 (SEQ ID NO:72), M90520 (SEQ ID NO:73), P03153 (SEQ ID NO:74), AF110999 (SEQ ID NO:75), and M95589 (SEQ ID NO:76), the disclosures of each of which are incorporated herein by reference. These HBcAg variants differ in amino acid sequence at a number of positions, including amino acid residues which corresponds to the amino acid residues located at positions 12, 13, 21, 22, 24, 29, 32, 33, 35, 38, 40, 42, 44, 45, 49, 51, 57, 58, 59, 64, 66, 67, 69, 74, 77, 80, 81, 87, 92, 93, 97, 98, 100, 103, 105, 106, 109, 113, 116, 121, 126, 130, 133, 135, 141, 147, 149, 157, 176, 178, 182 and 183 in SEQ ID NO:77. Further HBcAg variants suitable for use in the compositions of the invention, and which may be further modified according to the disclosure of this specification are described in WO 00/198333, WO 00/177158 and WO 00/214478.

HBcAgs suitable for use in the present invention can be derived from any organism so long as they are able to be coupled, fused or otherwise attached to, in particular as long as they are capable of packaging an antigen and induce an immune response.

As noted above, generally processed HBcAgs (i.e., those which lack leader sequences) will be used in the vaccine compositions of the invention. The present invention includes vaccine compositions, as well as methods for using these compositions, which employ the above described variant HBcAgs.

Further included within the scope of the invention are additional HBcAg variants which are capable of associating to form dimeric or multimeric structures. Thus, the invention further includes vaccine compositions comprising HBcAg polypeptides comprising, or alternatively consisting of, amino acid sequences which are at least 80%, 85%, 90%, 95%, 97% or 99% identical to any of the wild-type amino acid sequences, and forms of these proteins which have been processed, where appropriate, to remove the N-terminal leader sequence.

Whether the amino acid sequence of a polypeptide has an amino acid sequence that is at least 80%, 85%, 90%, 95%, 97% or 99% identical to one of the wild-type amino acid sequences, or a subportion thereof, can be determined conventionally using known computer programs such the Bestfit program. When using Bestfit or any other sequence alignment program to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed.

The HBcAg variants and precursors having the amino acid sequences set out in SEQ ID NOs: 29-72 and 73-76 are relatively similar to each other. Thus, reference to an amino acid residue of a HBcAg variant located at a position which corresponds to a particular position in SEQ ID NO:77, refers to the amino acid residue which is present at that position in the amino acid sequence shown in SEQ ID NO:77. The homology between these HBcAg variants is for the most part high enough among Hepatitis B viruses that infect mammals so that one skilled in the art would have little difficulty reviewing both the amino acid sequence shown in SEQ ID NO:77 and in FIG. 1, respectively, and that of a particular HBcAg variant and identifying “corresponding” amino acid residues. Furthermore, the HBcAg amino acid sequence shown in SEQ ID NO:73, which shows the amino acid sequence of a HBcAg derived from a virus which infect woodchucks, has enough homology to the HBcAg having the amino acid sequence shown in SEQ ID NO:77 that it is readily apparent that a three amino acid residue insert is present in SEQ ID NO:73 between amino acid residues 155 and 156 of SEQ ID NO:77.

The invention also includes vaccine compositions which comprise HBcAg variants of Hepatitis B viruses which infect birds, as wells as vaccine compositions which comprise fragments of these HBcAg variants. As one skilled in the art would recognize, one, two, three or more of the cysteine residues naturally present in these polypeptides could be either substituted with another amino acid residue or deleted prior to their inclusion in vaccine compositions of the invention.

As discussed above, the elimination of free cysteine residues reduces the number of sites where toxic components can bind to the HBcAg, and also eliminates sites where cross-linking of lysine and cysteine residues of the same or of neighboring HBcAg molecules can occur. Therefore, in another embodiment of the present invention, one or more cysteine residues of the Hepatitis B virus capsid protein have been either deleted or substituted with another amino acid residue.

In other embodiments, compositions and vaccine compositions, respectively, of the invention will contain HBcAgs from which the C-terminal region (e.g., amino acid residues 145-185 or 150-185 of SEQ ID NO: 77) has been removed. Thus, additional modified HBcAgs suitable for use in the practice of the present invention include C-terminal truncation mutants. Suitable truncation mutants include HBcAgs where 1, 5, 10, 15, 20, 25, 30, 34, 35, amino acids have been removed from the C-terminus.

HBcAgs suitable for use in the practice of the present invention also include N-terminal truncation mutants. Suitable truncation mutants include modified HBcAgs where 1, 2, 5, 7, 9, 10, 12, 14, 15, or 17 amino acids have been removed from the N-terminus.

Further HBcAgs suitable for use in the practice of the present invention include N- and C-terminal truncation mutants. Suitable truncation mutants include HBcAgs where 1, 2, 5, 7, 9, 10, 12, 14, 15, and 17 amino acids have been removed from the N-terminus and 1, 5, 10, 15, 20, 25, 30, 34, 35 amino acids have been removed from the C-terminus.

The invention further includes compositions and vaccine compositions, respectively, comprising HBcAg polypeptides comprising, or alternatively essentially consisting of, or alternatively consisting of, amino acid sequences which are at least 80%, 85%, 90%, 95%, 97%, or 99% identical to the above described truncation mutants.

In certain embodiments of the invention, a lysine residue is introduced into a HBcAg polypeptide, to mediate the binding of the antigen or antigenic determinant to the VLP of HBcAg. In preferred embodiments, compositions of the invention are prepared using a HBcAg comprising, or alternatively consisting of, amino acids 1-144, or 1-149, 1-185 of SEQ ID NO:77, which is modified so that the amino acids corresponding to positions 79 and 80 are replaced with a peptide having the amino acid sequence of Gly-Gly-Lys-Gly-Gly (SEQ ID NO:78). These compositions are particularly useful in those embodiments where an antigenic determinant is coupled to a VLP of HBcAg. In further preferred embodiments, the cysteine residues at positions 48 and 107 of SEQ ID NO:77 are mutated to serine. The invention further includes compositions comprising the corresponding polypeptides having amino acid sequences shown in any of SEQ ID NOs:29-74 which also have above noted amino acid alterations. Further included within the scope of the invention are additional HBcAg variants which are capable of associating to form a capsid or VLP and have the above noted amino acid alterations. Thus, the invention further includes compositions and vaccine compositions, respectively, comprising HBcAg polypeptides which comprise, or alternatively consist of, amino acid sequences which are at least 80%, 85%, 90%, 95%, 97% or 99% identical to any of the wild-type amino acid sequences, and forms of these proteins which have been processed, where appropriate, to remove the N-terminal leader sequence and modified with above noted alterations.

Compositions or vaccine compositions of the invention may comprise mixtures of different HBcAgs. Thus, these vaccine compositions may be composed of HBcAgs which differ in amino acid sequence. For example, vaccine compositions could be prepared comprising a “wild-type” HBcAg and a modified HBcAg in which one or more amino acid residues have been altered (e.g., deleted, inserted or substituted). Further, preferred vaccine compositions of the invention are those which present highly ordered and repetitive antigen arrays.

The inventive composition further comprises at least one antigen or antigenic determinant bound to the virus-like particle. The invention provides for compositions that vary according to the antigen or antigenic determinant selected in consideration of the desired therapeutic effect. Very preferred antigens or antigenic determinants suitable for use in the present invention are disclosed in WO 00/32227, in WO 01/85208 and in WO 02/056905, the disclosures of which are herewith incorporated by reference in their entirety.

The antigen can be any antigen of known or yet unknown provenance. It can be isolated from bacteria, viruses or other pathogens or can be a recombinant antigen obtained from expression of suitable nucleic acid coding therefor. In a preferred embodiment, the antigen is a recombinant antigen. The selection of the antigen is, of course, dependent upon the immunological response desired and the host.

In one embodiment of the immune enhancing composition of the present invention, the immune response is induced against the VLP itself. In another embodiment of the invention a virus-like particle is coupled, fused or otherwise attached to an antigen/immunogen against which an enhanced immune response is desired.

In a further preferred embodiment of the invention, the at least one antigen or antigenic determinant is fused to the virus-like particle. As outlined above, a VLP is typically composed of at least one subunit assembling into a VLP. Thus, in again a further preferred embodiment of the invention, the antigen or antigenic determinant is fused to at least one subunit of the virus-like particle or of a protein capable of being incorporated into a VLP generating a chimeric VLP-subunit-antigen fusion.

Fusion of the antigen or antigenic determinant can be effected by insertion into the VLP subunit sequence, or by fusion to either the N- or C-terminus of the VLP-subunit or protein capable of being incorporated into a VLP. Hereinafter, when referring to fusion proteins of a peptide to a VLP subunit, the fusion to either ends of the subunit sequence or internal insertion of the peptide within the subunit sequence are encompassed.

Fusion may also be effected by inserting antigen or antigenic determinant sequences into a variant of a VLP subunit where part of the subunit sequence has been deleted, that are further referred to as truncation mutants. Truncation mutants may have N- or C-terminal, or internal deletions of part of the sequence of the VLP subunit. For example, the specific VLP HBcAg with, for example, deletion of amino acid residues 79 to 81 is a truncation mutant with an internal deletion. Fusion of antigens or antigenic determinants to either the N- or C-terminus of the truncation mutants VLP-subunits also lead to embodiments of the invention. Likewise, fusion of an epitope into the sequence of the VLP subunit may also be effected by substitution, where for example for the specific VLP HBcAg, amino acids 79-81 are replaced with a foreign epitope. Thus, fusion, as referred to hereinafter, may be effected by insertion of the antigen or antigenic determinant sequence in the sequence of a VLP subunit, by substitution of part of the sequence of the VLP subunit with the antigen or antigenic determinant, or by a combination of deletion, substitution or insertions.

The chimeric antigen or antigenic determinant −VLP subunit will be in general capable of self-assembly into a VLP. VLP displaying epitopes fused to their subunits are also herein referred to as chimeric VLPs. As indicated, the virus-like particle comprises or alternatively is composed of at least one VLP subunit. In a further embodiment of the invention, the virus-like particle comprises or alternatively is composed of a mixture of chimeric VLP subunits and non-chimeric VLP subunits, i.e. VLP subunits not having an antigen fused thereto, leading to so called mosaic particles. This may be advantageous to ensure formation of, and assembly to a VLP. In those embodiments, the proportion of chimeric VLP-subunits may be 1, 2, 5, 10, 20, 30, 40, 50, 60, 70, 80, 90, 95% or higher.

Flanking amino acid residues may be added to either end of the sequence of the peptide or epitope to be fused to either end of the sequence of the subunit of a VLP, or for internal insertion of such peptidic sequence into the sequence of the subunit of a VLP. Glycine and serine residues are particularly favored amino acids to be used in the flanking sequences added to the peptide to be fused. Glycine residues confer additional flexibility, which may diminish the potentially destabilizing effect of fusing a foreign sequence into the sequence of a VLP subunit.

In a specific embodiment of the invention, the VLP is a Hepatitis B core antigen VLP. Fusion proteins of the antigen or antigenic determinant to either the N-terminus of a HBcAg (Neyrinck, S. et al., Nature Med. 5:1157-1163 (1999)) or insertions in the so called major immunodominant region (MIR) have been described (Pumpens, P. and Grens, E., Intervirology 44:98-114 (2001)), WO 01/98333), and are preferred embodiments of the invention. Naturally occurring variants of HBcAg with deletions in the MIR have also been described (Pumpens, P. and Grens, E., Intervirology 44:98-114 (2001), which is expressly incorporated by reference in its entirety), and fusions to the N- or C-terminus, as well as insertions at the position of the MIR corresponding to the site of deletion as compared to a wt HBcAg are further embodiments of the invention. Fusions to the C-terminus have also been described (Pumpens, P. and Grens, E., Intervirology 44:98-114 (2001)). One skilled in the art will easily find guidance on how to construct fusion proteins using classical molecular biology techniques (Sambrook, J. et al., eds., Molecular Cloning, A Laboratory Manual, 2nd. edition, Cold Spring Habor Laboratory Press, Cold Spring Harbor, N.Y. (1989), Ho et al., Gene 77:51 (1989)). Vectors and plasmids encoding HBcAg and HBcAg fusion proteins and useful for the expression of a HBcAg and HBcAg fusion proteins have been described (Pumpens, P. & Grens, E. Intervirology 44: 98-114 (2001), Neyrinck, S. et al., Nature Med. 5:1157-1163 (1999)) and can be used in the practice of the invention. An important factor for the optimization of the efficiency of self-assembly and of the display of the epitope to be inserted in the MIR of HBcAg is the choice of the insertion site, as well as the number of amino acids to be deleted from the HBcAg sequence within the MIR (Pumpens, P. and Grens, E., Intervirology 44:98-114 (2001); EP 0 421 635; U.S. Pat. No. 6,231,864) upon insertion, or in other words, which amino acids form HBcAg are to be substituted with the new epitope. For example, substitution of HBcAg amino acids 76-80, 79-81, 79-80, 75-85 or 80-81 with foreign epitopes has been described (Pumpens, P. and Grens, E., Intervirology 44:98-114 (2001); EP0421635; U.S. Pat. No. 6,231,864). HBcAg contains a long arginine tail (Pumpens, P. and Grens, E., Intervirology 44:98-114 (2001)) which is dispensable for capsid assembly and capable of binding nucleic acids (Pumpens, P. and Grens, E., Intervirology 44:98-114 (2001)). HBcAg either comprising or lacking this arginine tail are both embodiments of the invention.

In a further preferred embodiment of the invention, the VLP is a VLP of a RNA phage. The major coat proteins of RNA phages spontaneously assemble into VLPs upon expression in bacteria, and in particular in E. coli. Specific examples of bacteriophage coat proteins which can be used to prepare compositions of the invention include the coat proteins of RNA bacteriophages such as bacteriophage Qβ (SEQ ID NO:10; PIR Database, Accession No. VCBPQβ referring to Qβ CP and SEQ ID NO: 11; Accession No. AAA16663 referring to Qβ A1 protein) and bacteriophage fr (SEQ ID NO: 13; PIR Accession No. VCBPFR).

In a more preferred embodiment, the at least one antigen or antigenic determinant is fused to a Qβ coat protein. Fusion protein constructs wherein epitopes have been fused to the C-terminus of a truncated form of the A1 protein of Qβ, or inserted within the A1 protein have been described (Kozlovska, T. M., et al., Intervirology, 39:9-15 (1996)). The A1 protein is generated by suppression at the UGA stop codon and has a length of 329 aa, or 328 aa, if the cleavage of the N-terminal methionine is taken into account. Cleavage of the N-terminal methionine before an alanine (the second amino acid encoded by the Qβ CP gene) usually takes place in E. coli, and such is the case for N-termini of the Qβ coat proteins. The part of the A1 gene, 3′ of the UGA amber codon encodes the CP extension, which has a length of 195 amino acids. Insertion of the at least one antigen or antigenic determinant between position 72 and 73 of the CP extension leads to further embodiments of the invention (Kozlovska, T. M., et al., Intervirology 39:9-15 (1996)). Fusion of an antigen or antigenic determinant at the C-terminus of a C-terminally truncated Qβ A1 protein leads to further preferred embodiments of the invention. For example, Kozlovska et al., (Intervirology, 39: 9-15 (1996)) describe Qβ A1 protein fusions where the epitope is fused at the C-terminus of the Qβ CP extension truncated at position 19.

As described by Kozlovska et al. (Intervirology, 39: 9-15 (1996)), assembly of the particles displaying the fused epitopes typically requires the presence of both the A1 protein-antigen fusion and the wt CP to form a mosaic particle. However, embodiments comprising virus-like particles, and hereby in particular the VLPs of the RNA phage Qβ coat protein, which are exclusively composed of VLP subunits having at least one antigen or antigenic determinant fused thereto, are also within the scope of the present invention.

The production of mosaic particles may be effected in a number of ways. Kozlovska et al., Intervirology, 39:9-15 (1996), describe three methods, which all can be used in the practice of the invention. In the first approach, efficient display of the fused epitope on the VLPs is mediated by the expression of the plasmid encoding the Qβ A1 protein fusion having a UGA stop codong between CP and CP extension in a E. coli strain harboring a plasmid encoding a cloned UGA suppressor tRNA which leads to translation of the UGA codon into Trp (pISM3001 plasmid (Smiley B. K., et al., Gene 134:33-40 (1993))). In another approach, the CP gene stop codon is modified into UAA, and a second plasmid expressing the A1 protein-antigen fusion is cotransformed. The second plasmid encodes a different antibiotic resistance and the origin of replication is compatible with the first plasmid (Kozlovska, T. M., et al., Intervirology 39:9-15 (1996)). In a third approach, CP and the A1 protein-antigen fusion are encoded in a bicistronic manner, operatively linked to a promoter such as the Trp promoter, as described in FIG. 1 of Kozlovska et al., Intervirology, 39:9-15 (1996).

In a further embodiment, the antigen or antigenic determinant is inserted between amino acid 2 and 3 (numbering of the cleaved CP, that is wherein the N-terminal methionine is cleaved) of the fr CP, thus leading to an antigen or antigenic determinant −fr CP fusion protein. Vectors and expression systems for construction and expression of fr CP fusion proteins self-assembling to VLP and useful in the practice of the invention have been described (Pushko P. et al., Prot. Eng. 6:883-891 (1993)). In a specific embodiment, the antigen or antigenic determinant sequence is inserted into a deletion variant of the fr CP after amino acid 2, wherein residues 3 and 4 of the fr CP have been deleted (Pushko P. et al., Prot. Eng. 6:883-891 (1993)).

Fusion of epitopes in the N-terminal protuberant β-hairpin of the coat protein of RNA phage MS-2 and subsequent presentation of the fused epitope on the self-assembled VLP of RNA phage MS-2 has also been described (WO 92/13081), and fusion of an antigen or antigenic determinant by insertion or substitution into the coat protein of MS-2 RNA phage is also falling under the scope of the invention.

In another embodiment of the invention, the antigen or antigenic determinant is fused to a capsid protein of papillomavirus. In a more specific embodiment, the antigen or antigenic determinant is fused to the major capsid protein L1 of bovine papillomavirus type 1 (BPV-1). Vectors and expression systems for construction and expression of BPV-1 fusion proteins in a baculovirus/insect cells systems have been described (Chackerian, B. et al., Proc. Natl. Acad. Sci. USA 96:2373-2378 (1999); WO 00/23955). Substitution of amino acids 130-136 of BPV-1 L1 with an antigen or antigenic determinant leads to a BPV-1 L1-antigen fusion protein, which is a preferred embodiment of the invention. Cloning in a baculovirus vector and expression in baculovirus infected Sf9 cells has been described, and can be used in the practice of the invention (Chackerian, B. et al., Proc. Natl. Acad. Sci. USA 96:2373-2378 (1999); WO 00/23955). Purification of the assembled particles displaying the fused antigen or antigenic determinant can be performed in a number of ways, such as for example gel filtration or sucrose gradient ultracentrifugation (Chackerian, B. et al., Proc. Natl. Acad. Sci. USA 96:2373-2378 (1999), WO 00/23955).

In a further embodiment of the invention, the antigen or antigenic determinant is fused to a Ty protein capable of being incorporated into a Ty VLP. In a more specific embodiment, the antigen or antigenic determinant is fused to the p1 or capsid protein encoded by the TYA gene (Roth, J. F., Yeast 16:785-795 (2000)). The yeast retrotransposons Ty1, 2, 3 and 4 have been isolated from Saccharomyces Serevisiae, while the retrotransposon Tf1 has been isolated from Schizosaccharomyces Pombae (Boeke, J. D. and Sandmeyer, S. B., “Yeast Transposable elements,” in The molecular and Cellular Biology of the Yeast Saccharomyces: Genome dynamics, Protein Synthesis, and Energetics, p. 193, Cold Spring Harbor Laboratory Press (1991)). The retrotransposons Ty1 and 2 are related to the copia class of plant and animal elements, while Ty3 belongs to the gypsy family of retrotransposons, which is related to plants and animal retroviruses. In the Ty1 retrotransposon, the p1 protein, also referred to as Gag or capsid protein, has a length of 440 amino acids. P1 is cleaved during maturation of the VLP at position 408, leading to the p2 protein, the essential component of the VLP.

Fusion proteins to p1 and vectors for the expression of said fusion proteins in Yeast have been described (Adams, S. E., et al., Nature 329:68-70 (1987)). So, for example, an antigen or antigenic determinant may be fused to p1 by inserting a sequence coding for the antigen or antigenic determinant into the BamH1 site of the pMA5620 plasmid (Adams, S. E., et al., Nature 329:68-70 (1987)). The cloning of sequences coding for foreign epitopes into the pMA5620 vector leads to expression of fusion proteins comprising amino acids 1-381 of p1 of Ty1-15, fused C-terminally to the N-terminus of the foreign epitope. Likewise, N-terminal fusion of an antigen or antigenic determinant, or internal insertion into the p1 sequence, or substitution of part of the p1 sequence are also meant to fall within the scope of the invention. In particular, insertion of an antigen or antigenic determinant into the Ty sequence between amino acids 30-31, 67-68, 113-114 and 132-133 of the Ty protein p1 (EP0677111) leads to preferred embodiments of the invention.

Further VLPs suitable for fusion of antigens or antigenic determinants are, for example, Retrovirus-like-particles (WO9630523), HIV2 Gag (Kang, Y. C., et al, Biol. Chem. 380:353-364 (1999)), Cowpea Mosaic Virus (Taylor, K. M. et al., Biol. Chem. 380:387-392 (1999)), parvovirus VP2 VLP (Rueda, P. et al., Virology 263:89-99 (1999)), HBsAg (U.S. Pat. No. 4,722,840, EP0020416B1).

Examples of chimeric VLPs suitable for the practice of the invention are also those described in Intervirology 39:1 (1996). Further examples of VLPs contemplated for use in the invention are: HPV-1, HPV-6, HPV-11, HPV-16, HPV-18, HPV-33, HPV-45, CRPV, COPV, HIV GAG, Tobacco Mosaic Virus. Virus-like particles of SV-40, Polyomavirus, Adenovirus, Herpes Simplex Virus, Rotavirus and Norwalk virus have also been made, and chimeric VLPs of those VLPs comprising an antigen or antigenic determinant are also within the scope of the present invention.

As indicated, embodiments comprising antigens fused to the virus-like particle by insertion within the sequence of the virus-like particle building monomer are also within the scope of the present invention. In some cases, antigens can be inserted in a form of the virus-like particle building monomer containing deletions. In these cases, the virus-like particle building monomer may not be able to form virus-like structures in the absence of the inserted antigen.

In the immune enhancing composition of the invention a virus-like particle is coupled, fused or otherwise attached to an antigen/immunogen against which an enhanced immune response is desired.

In some instances, recombinant DNA technology can be utilized to fuse a heterologous protein to a VLP protein (Kratz, P. A., et al., Proc. Natl. Acad. Sci. USA 96:1915 (1999)). For example, the present invention encompasses VLPs recombinantly fused or chemically conjugated (including both covalently and non-covalently conjugations) to an antigen (or portion thereof, preferably at least 10, 20 or 50 amino acids) of the present invention to generate fusion proteins or conjugates. The fusion does not necessarily need to be direct, but can occur through linker sequences. More generally, in the case that epitopes, either fused, conjugated or otherwise attached to the virus-like particle, are used as antigens in accordance with the invention, spacer or linker sequences are typically added at one or both ends of the epitopes. Such linker sequences preferably comprise sequences recognized by the proteasome, proteases of the endosomes or other vesicular compartment of the cell.

One way of coupling is by a peptide bond, in which the conjugate can be a contiguous polypeptide, i.e. a fusion protein. In a fusion protein according to the present invention, different peptides or polypeptides are linked in frame to each other to form a contiguous polypeptide. Thus a first portion of the fusion protein comprises an antigen or immunogen and a second portion of the fusion protein, either N-terminal or C-terminal to the first portion, comprises a VLP. Alternatively, internal insertion into the VLP, with optional linking sequences on both ends of the antigen, can also be used in accordance with the present invention.

When HBcAg is used as the VLP, it is preferred that the antigen is linked to the C-terminal end of the HBcAg particle. The hepatitis B core antigen (HBcAg) exhibiting a C-terminal fusion of the MHC class I restricted peptide p33 derived from lymphocytic choriomeningitis virus (LCVM) glycoprotein was used a model antigen (HBcAg-p33). The 183 amino acids long wild type HBc protein assembles into highly structured particles composed of 180 subunits assuming icosahedral geometry. The flexibility of the HBcAg and other VLPs in accepting relatively large insertions of foreign sequences at different positions while retaining the capacity to form structured capsids is well documented in the literature. This makes the HBc VLPs attractive candidates for the design of non-replicating vaccines.

A flexible linker sequence (e.g. a polyglycine/polyserine-containing sequence such as [Gly₄ Ser]₂ (Huston et al., Meth. Enzymol 203:46-88 (1991)) can be inserted into the fusion protein between the antigen and ligand. Also, the fusion protein can be constructed to contain an “epitope tag”, which allows the fusion protein to bind an antibody (e.g. monoclonal antibody) for example for labeling or purification purposes. An example of an epitope tag is a Glu-Glu-Phe tripeptide which is recognized by the monoclonal antibody YL1/2.

The invention also relates to the chimeric DNA which contains a sequence coding for the VLP and a sequence coding for the antigen/immunogen. The DNA can be expressed, for example, in insect cells transformed with Baculoviruses, in yeast or in bacteria. There are no restrictions regarding the expression system, of which a large selection is available for routine use. Preferably, a system is used which allows expression of the proteins in large amounts. In general, bacterial expression systems are preferred on account of their efficiency. One example of a bacterial expression system suitable for use within the scope of the present invention is the one described by Clarke et al., J. Gen. Virol. 71: 1109-1117 (1990); Borisova et al., J. Virol. 67: 3696-3701 (1993); and Studier et al., Methods Enzymol. 185:60-89 (1990). An example of a suitable yeast expression system is the one described by Emr, Methods Enzymol. 185:231-3 (1990); Baculovirus systems, which have previously been used for preparing capsid proteins, are also suitable. Constitutive or inducible expression systems can be used. By the choice and possible modification of available expression systems it is possible to control the form in which the proteins are obtained.

In a specific embodiment of the invention, the antigen to which an enhanced immune response is desired is coupled, fused or otherwise attached in frame to the Hepatitis B virus capsid (core) protein (HBcAg). However, it will be clear to all individuals in the art that other virus-like particles can be utilized in the fusion protein construct of the invention.

In a further preferred embodiment of the present invention, the at least one antigen or antigenic determinant is bound to the virus-like particle by at least one covalent bond. Preferably, the least one antigen or antigenic determinant is bound to the virus-like particle by at least one covalent bond, said covalent bond being a non-peptide bond leading to an antigen or antigenic determinant array and antigen or antigenic determinant −VLP conjugate, respectively. This antigen or antigenic determinant array and conjugate, respectively, has typically and preferably a repetitive and ordered structure since the at least one antigen or antigenic determinant is bound to the VLP in an oriented manner. The formation of a repetitive and ordered antigen or antigenic determinant −VLP array and conjugate, respectively, is ensured by an oriented and directed as well as defined binding and attachment, respectively, of the at least one antigen or antigenic determinant to the VLP as will become apparent in the following. Furthermore, the typical inherent highly repetitive and organized structure of the VLPs advantageously contributes to the display of the antigen or antigenic determinant in a highly ordered and repetitive fashion leading to a highly organized and repetitive antigen or antigenic determinant −VLP array and conjugate, respectively.

Therefore, the preferred inventive conjugates and arrays, respectively, differ from prior art conjugates in their highly organized structure, dimensions, and in the repetitiveness of the antigen on the surface of the array. The preferred embodiment of this invention, furthermore, allows expression of the particle in an expression host guaranteeing proper folding and assembly of the VLP, to which the antigen is then further coupled

The present invention discloses methods of binding of antigen or antigenic determinant to VLPs. As indicated, in one aspect of the invention, the at least one antigen or antigenic determinant is bound to the VLP by way of chemical cross-linking, typically and preferably by using a heterobifunctional cross-linker. Several hetero-bifunctional cross-linkers are known to the art. In preferred embodiments, the hetero-bifunctional cross-linker contains a functional group which can react with preferred first attachment sites, i.e. with the side-chain amino group of lysine residues of the VLP or at least one VLP subunit, and a further functional group which can react with a preferred second attachment site, i.e. a cysteine residue fused to the antigen or antigenic determinant and optionally also made available for reaction by reduction. The first step of the procedure, typically called the derivatization, is the reaction of the VLP with the cross-linker. The product of this reaction is an activated VLP, also called activated carrier. In the second step, unreacted cross-linker is removed using usual methods such as gel filtration or dialysis. In the third step, the antigen or antigenic determinant is reacted with the activated VLP, and this step is typically called the coupling step. Unreacted antigen or antigenic determinant may be optionally removed in a fourth step, for example by dialysis. Several hetero-bifunctional cross-linkers are known to the art. These include the preferred cross-linkers SMPH (Pierce), Sulfo-MBS, Sulfo-EMCS, Sulfo-GMBS, Sulfo-SIAB, Sulfo-SMPB, Sulfo-SMCC, SVSB, SIA and other cross-linkers available for example from the Pierce Chemical Company (Rockford, Ill., USA), and having one functional group reactive towards amino groups and one functional group reactive towards cysteine residues. The above mentioned cross-linkers all lead to formation of a thioether linkage. Another class of cross-linkers suitable in the practice of the invention is characterized by the introduction of a disulfide linkage between the antigen or antigenic determinant and the VLP upon coupling. Preferred cross-linkers belonging to this class include for example SPDP and Sulfo-LC-SPDP (Pierce). The extent of derivatization of the VLP with cross-linker can be influenced by varying experimental conditions such as the concentration of each of the reaction partners, the excess of one reagent over the other, the pH, the temperature and the ionic strength. The degree of coupling, i.e. the amount of antigens or antigenic determinants per subunits of the VLP can be adjusted by varying the experimental conditions described above to match the requirements of the vaccine.

A particularly favored method of binding of antigens or antigenic determinants to the VLP, is the linking of a lysine residue on the surface of the VLP with a cysteine residue on the antigen or antigenic determinant. In some embodiments, fusion of an amino acid linker containing a cysteine residue, as a second attachment site or as a part thereof, to the antigen or antigenic determinant for coupling to the VLP may be required.

In general, flexible amino acid linkers are favored. Examples of the amino acid linker are selected from the group consisting of: (a) CGG; (b) N-terminal gamma 1-linker; (c) N-terminal gamma 3-linker; (d) Ig hinge regions; (e) N-terminal glycine linkers; (f) (G)ₖC(G)ₙ with n=0-12 and k=0-5; (g) N-terminal glycine-serine linkers; (h) (G)ₖC(G)ₘ(S)ₗ(GGGGS)ₙ with n=0-3, k=0-5, m=0-10, l=0-2; (i) GGC; (k) GGC-NH2; (l) C-terminal gamma 1-linker; (m) C-terminal gamma 3-linker; (n) C-terminal glycine linkers; (o) (G)ₙC(G)ₖ with n=0-12 and k=0-5; (p) C-terminal glycine-serine linkers; (q) (G)ₘ(S)ₗ(GGGGS)ₙ(G)ₒC(G)ₖ with n=0-3, k=0-5, m=0-10, l=0-2, and o=0-8.

Further examples of amino acid linkers are the hinge region of Immunoglobulins, glycine serine linkers (GGGGS)ₙ, and glycine linkers (G)ₙ all further containing a cysteine residue as second attachment site and optionally further glycine residues. Typically preferred examples of said amino acid linkers are N-terminal gamma1: CGDKTHTSPP; C-terminal gamma 1: DKTHTSPPCG; N-terminal gamma 3: CGGPKPSTPPGSSGGAP; C-terminal gamma 3: PKPSTPPGSSGGAPGGCG; N-terminal glycine linker: GCGGGG and C-terminal glycine linker: GGGGCG.

Other amino acid linkers particularly suitable in the practice of the invention, when a hydrophobic antigen or antigenic determinant is bound to a VLP, are CGKKGG, or CGDEGG for N-terminal linkers, or GGKKGC and GGEDGC, for the C-terminal linkers. For the C-terminal linkers, the terminal cysteine is optionally C-terminally amidated.

In preferred embodiments of the present invention, GGCG, GGC or GGC-NH2 (“NH2” stands for amidation) linkers at the C-terminus of the peptide or CGG at its N-terminus are preferred as amino acid linkers. In general, glycine residues will be inserted between bulky amino acids and the cysteine to be used as second attachment site, to avoid potential steric hindrance of the bulkier amino acid in the coupling reaction. In the most preferred embodiment of the invention, the amino acid linker GGC-NH2 is fused to the C-terminus of the antigen or antigenic determinant.

The cysteine residue present on the antigen or antigenic determinant has to be in its reduced state to react with the hetero-bifunctional cross-linker on the activated VLP, that is a free cysteine or a cysteine residue with a free sulfhydryl group has to be available. In the instance where the cysteine residue to function as binding site is in an oxidized form, for example if it is forming a disulfide bridge, reduction of this disulfide bridge with e.g. DTT, TCEP or β-mercaptoethanol is required. Low concentrations of reducing agent are compatible with coupling as described in WO 02/05690, higher concentrations inhibit the coupling reaction, as a skilled artisan would know, in which case the reductand has to be removed or its concentration decreased prior to coupling, e.g. by dialysis, gel filtration or reverse phase HPLC.

Binding of the antigen or antigenic determinant to the VLP by using a hetero-bifunctional cross-linker according to the preferred methods described above, allows coupling of the antigen or antigenic determinant to the VLP in an oriented fashion. Other methods of binding the antigen or antigenic determinant to the VLP include methods wherein the antigen or antigenic determinant is cross-linked to the VLP using the carbodiimide EDC, and NHS. In further methods, the antigen or antigenic determinant is attached to the VLP using a homo-bifunctional cross-linker such as glutaraldehyde, DSG, BM[PEO]₄, BS³, (Pierce Chemical Company, Rockford, Ill., USA) or other known homo-bifunctional cross-linkers with functional groups reactive towards amine groups or carboxyl groups of the VLP.

Other methods of binding the VLP to an antigen or antigenic determinant include methods where the VLP is biotinylated, and the antigen or antigenic determinant expressed as a streptavidin-fusion protein, or methods wherein both the antigen or antigenic determinant and the VLP are biotinylated, for example as described in WO 00/23955. In this case, the antigen or antigenic determinant may be first bound to streptavidin or avidin by adjusting the ratio of antigen or antigenic determinant to streptavidin such that free binding sites are still available for binding of the VLP, which is added in the next step. Alternatively, all components may be mixed in a “one pot” reaction. Other ligand-receptor pairs, where a soluble form of the receptor and of the ligand is available, and are capable of being cross-linked to the VLP or the antigen or antigenic determinant, may be used as binding agents for binding antigen or antigenic determinant to the VLP. Alternatively, either the ligand or the receptor may be fused to the antigen or antigenic determinant, and so mediate binding to the VLP chemically bound or fused either to the receptor, or the ligand respectively. Fusion may also be effected by insertion or substitution.

As already indicated, in a favored embodiment of the present invention, the VLP is the VLP of a RNA phage, and in a more preferred embodiment, the VLP is the VLP of RNA phage Qβ coat protein.

One or several antigen molecules, i.e. one or several antigens or antigenic determinants, can be attached to one subunit of the capsid or VLP of RNA phages coat proteins, preferably through the exposed lysine residues of the VLP of RNA phages, if sterically allowable. A specific feature of the VLP of the coat protein of RNA phages and in particular of the Qβ coat protein VLP is thus the possibility to couple several antigens per subunit. This allows for the generation of a dense antigen array.

In a preferred embodiment of the invention, the binding and attachment, respectively, of the at least one antigen or antigenic determinant to the virus-like particle is by way of interaction and association, respectively, between at least one first attachment site of the virus-like particle and at least one second attachment of the antigen or antigenic determinant.

VLPs or capsids of Qβ coat protein display a defined number of lysine residues on their surface, with a defined topology with three lysine residues pointing towards the interior of the capsid and interacting with the RNA, and four other lysine residues exposed to the exterior of the capsid. These defined properties favor the attachment of antigens to the exterior of the particle, rather than to the interior of the particle where the lysine residues interact with RNA. VLPs of other RNA phage coat proteins also have a defined number of lysine residues on their surface and a defined topology of these lysine residues.

In further preferred embodiments of the present invention, the first attachment site is a lysine residue and/or the second attachment comprises sulfhydryl group or a cysteine residue. In a very preferred embodiment of the present invention, the first attachment site is a lysine residue and the second attachment is a cysteine residue.

In very preferred embodiments of the invention, the antigen or antigenic determinant is bound via a cysteine residue, to lysine residues of the VLP of RNA phage coat protein, and in particular to the VLP of Qβ coat protein.

Another advantage of the VLPs derived from RNA phages is their high expression yield in bacteria that allows production of large quantities of material at affordable cost.

As indicated, the inventive conjugates and arrays, respectively, differ from prior art conjugates in their highly organized structure, dimensions, and in the repetitiveness of the antigen on the surface of the array. Moreover, the use of the VLPs as carriers allow the formation of robust antigen arrays and conjugates, respectively, with variable antigen density. In particular, the use of VLPs of RNA phages, and hereby in particular the use of the VLP of RNA phage Qβ coat protein allows to achieve very high epitope density. In particular, a density of more than 1.5 epitopes per subunit could be reached by coupling the human Aβ1-6 peptide to the VLP of Qβ coat protein. The preparation of compositions of VLPs of RNA phage coat proteins with a high epitope density can be effected using the teaching of this application. In prefered embodiment of the invention, when an antigen or antigenic determinant is coupled to the VLP of Qβ coat protein, an average number of antigen or antigenic determinant per subunit of 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4 2.5, 2.6, 2.7, 2.8, 2.9, or higher is preferred.

The second attachment site, as defined herein, may be either naturally or non-naturally present with the antigen or the antigenic determinant. In the case of the absence of a suitable natural occurring second attachment site on the antigen or antigenic determinant, then a non-natural second attachment has to be engineered to the antigen.

As described above, four lysine residues are exposed on the surface of the VLP of Qβ coat protein. Typically these residues are derivatized upon reaction with a cross-linker molecule. In the instance where not all of the exposed lysine residues can be coupled to an antigen, the lysine residues which have reacted with the cross-linker are left with a cross-linker molecule attached to the ε-amino group after the derivatization step. This leads to disappearance of one or several positive charges, which may be detrimental to the solubility and stability of the VLP. By replacing some of the lysine residues with arginines, as in the disclosed Qβ coat protein mutants described below, we prevent the excessive disappearance of positive charges since the arginine residues do not react with the cross-linker. Moreover, replacement of lysine residues by arginines may lead to more defined antigen arrays, as fewer sites are available for reaction to the antigen.

Accordingly, exposed lysine residues were replaced by arginines in the following Qβ coat protein mutants and mutant Qβ VLPs disclosed in this application: Qβ-240 (Lys13-Arg; SEQ ID NO:23), Qβ-250 (Lys 2-Arg, Lys13-Arg; SEQ ID NO: 25) and Qβ-259 (Lys 2-Arg, Lys16-Arg; SEQ ID NO:27). The constructs were cloned, the proteins expressed, the VLPs purified and used for coupling to peptide and protein antigens. Qβ-251; (SEQ ID NO: 26) was also constructed, and guidance on how to express, purify and couple the VLP of Qβ-251 coat protein can be found throughout the application.

In a further embodiment, we disclose a Qβ mutant coat protein with one additional lysine residue, suitable for obtaining even higher density arrays of antigens. This mutant Qβ coat protein, Qβ-243 (Asn 10-Lys; SEQ ID NO: 24), was cloned, the protein expressed, and the capsid or VLP isolated and purified, showing that introduction of the additional lysine residue is compatible with self-assembly of the subunits to a capsid or VLP. Thus, antigen or antigenic determinant arrays and conjugates, respectively, may be prepared using VLP of Qβ coat protein mutants. A particularly favored method of attachment of antigens to VLPs, and in particular to VLPs of RNA phage coat proteins is the linking of a lysine residue present on the surface of the VLP of RNA phage coat proteins with a cysteine residue added to the antigen. In order for a cysteine residue to be effective as second attachment site, a sulfhydryl group must be available for coupling. Thus, a cysteine residue has to be in its reduced state, that is, a free cysteine or a cysteine residue with a free sulfhydryl group has to be available. In the instant where the cysteine residue to function as second attachment site is in an oxidized form, for example if it is forming a disulfide bridge, reduction of this disulfide bridge with e.g. DTT, TCEP or β-mercaptoethanol is required. The concentration of reductand, and the molar excess of reductand over antigen has to be adjusted for each antigen. A titration range, starting from concentrations as low as 10 μM or lower, up to 10 to 20 mM or higher reductand if required is tested, and coupling of the antigen to the carrier assessed. Although low concentrations of reductand are compatible with the coupling reaction as described in WO 02/056905, higher concentrations inhibit the coupling reaction, as a skilled artisan would know, in which case the reductand has to be removed or its concentration decreased, e.g. by dialysis, gel filtration or reverse phase HPLC. Advantageously, the pH of the dialysis or equilibration buffer is lower than 7, preferably 6. The compatibility of the low pH buffer with antigen activity or stability has to be tested.

Epitope density on the VLP of RNA phage coat proteins can be modulated by the choice of cross-linker and other reaction conditions. For example, the cross-linkers Sulfo-GMBS and SMPH typically allow reaching high epitope density. Derivatization is positively influenced by high concentration of reactands, and manipulation of the reaction conditions can be used to control the number of antigens coupled to VLPs of RNA phage coat proteins, and in particular to VLPs of Qβ coat protein.

Prior to the design of a non-natural second attachment site the position at which it should be fused, inserted or generally engineered has to be chosen. The selection of the position of the second attachment site may, by way of example, be based on a crystal structure of the antigen. Such a crystal structure of the antigen may provide information on the availability of the C- or N-termini of the molecule (determined for example from their accessibility to solvent), or on the exposure to solvent of residues suitable for use as second attachment sites, such as cysteine residues. Exposed disulfide bridges, as is the case for Fab fragments, may also be a source of a second attachment site, since they can be generally converted to single cysteine residues through mild reduction, with e.g. 2-mercaptoethylamine, TCEP, β-mercaptoethanol or DTT. Mild reduction conditions not affecting the immunogenicity of the antigen will be chosen. In general, in the case where immunization with a self-antigen is aiming at inhibiting the interaction of this self-antigen with its natural ligands, the second attachment site will be added such that it allows generation of antibodies against the site of interaction with the natural ligands. Thus, the location of the second attachment site will be selected such that steric hindrance from the second attachment site or any amino acid linker containing the same is avoided. In further embodiments, an antibody response directed at a site distinct from the interaction site of the self-antigen with its natural ligand is desired. In such embodiments, the second attachment site may be selected such that it prevents generation of antibodies against the interaction site of the self-antigen with its natural ligands.

Other criteria in selecting the position of the second attachment site include the oligomerization state of the antigen, the site of oligomerization, the presence of a cofactor, and the availability of experimental evidence disclosing sites in the antigen structure and sequence where modification of the antigen is compatible with the function of the self-antigen, or with the generation of antibodies recognizing the self-antigen.

In very preferred embodiments, the antigen or antigenic determinant comprises a single second attachment site or a single reactive attachment site capable of association with the first attachment sites on the core particle and the VLPs or VLP subunits, respectively. This further ensures a defined and uniform binding and association, respectively, of the at least one, but typically more than one, preferably more than 10, 20, 40, 80, 120 antigens to the core particle and VLP, respectively. The provision of a single second attachment site or a single reactive attachment site on the antigen, thus, ensures a single and uniform type of binding and association, respectively leading to a very highly ordered and repetitive array. For example, if the binding and association, respectively, is effected by way of a lysine- (as the first attachment site) and cysteine- (as a second attachment site) interaction, it is ensured, in accordance with this preferred embodiment of the invention, that only one cysteine residue per antigen, independent whether this cysteine residue is naturally or non-naturally present on the antigen, is capable of binding and associating, respectively, with the VLP and the first attachment site of the core particle, respectively.

In some embodiments, engineering of a second attachment site onto the antigen require the fusion of an amino acid linker containing an amino acid suitable as second attachment site according to the disclosures of this invention. Therefore, in a preferred embodiment of the present invention, an amino acid linker is bound to the antigen or the antigenic determinant by way of at least one covalent bond. Preferably, the amino acid linker comprises, or alternatively consists of, the second attachment site. In a further preferred embodiment, the amino acid linker comprises a sulfhydryl group or a cysteine residue. In another preferred embodiment, the amino acid linker is cysteine. Some criteria of selection of the amino acid linker as well as further preferred embodiments of the amino acid linker according to the invention have already been mentioned above.

In another specific embodiment of the invention, the attachment site is selected to be a lysine or cysteine residue that is fused in frame to the HBcAg. In a preferred embodiment, the antigen is fused to the C-terminus of HBcAg via a linker.

When an antigen or antigenic determinant is linked to the VLP through a lysine residue, it may be advantageous to either substitute or delete one or more of the naturally resident lysine residues, as well as other lysine residues present in HBcAg variants. The elimination of these lysine residues results in the removal of binding sites for antigens or antigenic determinants which could disrupt the ordered array and should improve the quality and uniformity of the final vaccine composition.

In many instances, when the naturally resident lysine residues are eliminated, another lysine will be introduced into the HBcAg as an attachment site for an antigen or antigenic determinant. Methods for inserting such a lysine residue are known in the art. Lysine residues may also be added without removing existing lysine residues.

The C-terminus of the HBcAg has been shown to direct nuclear localization of this protein. (Eckhardt et al., J. Virol. 65:575-582 (1991)). Further, this region of the protein is also believed to confer upon the HBcAg the ability to bind nucleic acids.

As indicated, HBcAgs suitable for use in the practice of the present invention also include N-terminal truncation mutants. Suitable truncation mutants include modified HBcAgs where 1, 2, 5, 7, 9, 10, 12, 14, 15, or 17 amino acids have been removed from the N-terminus. However, variants of virus-like particles containing internal deletions within the sequence of the subunit composing the virus-like particle are also suitable in accordance with the present invention, provided their compatibility with the ordered or particulate structure of the virus-like particle. For example, internal deletions within the sequence of the HBcAg are suitable (Preikschat, P., et al., J. Gen. Virol. 80:1777-1788 (1999)).

Further HBcAgs suitable for use in the practice of the present invention include N- and C-terminal truncation mutants. Suitable truncation mutants include HBcAgs where 1, 2, 5, 7, 9, 10, 12, 14, 15, and 17 amino acids have been removed from the N-terminus and 1, 5, 10, 15, 20, 25, 30, 34, 35, 36, 37, 38, 39 40, 41, 42 or 48 amino acids have been removed from the C-terminus.

Vaccine compositions of the invention can comprise mixtures of different HBcAgs. Thus, these vaccine compositions can be composed of HBcAgs which differ in amino acid sequence. For example, vaccine compositions could be prepared comprising a “wild-type” HBcAg and a modified HBcAg in which one or more amino acid residues have been altered (e.g., deleted, inserted or substituted). In most applications, however, only one type of a HBcAg will be used.

The present invention is applicable to a wide variety of antigens. In a preferred embodiment, the antigen is a protein, polypeptide or peptide. In another embodiment the antigen is DNA. The antigen can also be a lipid, a carbohydrate, or an organic molecule, in particular a small organic molecule such as nicotine.

Antigens of the invention can be selected from the group consisting of the following: (a) polypeptides suited to induce an immune response against cancer cells; (b) polypeptides suited to induce an immune response against infectious diseases; (c) polypeptides suited to induce an immune response against allergens; (d) polypeptides suited to induce an immune response in farm animals or pets; and (e) fragments (e.g., a domain) of any of the polypeptides set out in (a)-(d).

Preferred antigens include those from a pathogen (e.g. virus, bacterium, parasite, fungus) and tumors (especially tumor-associated antigens or “tumor markers”). Other preferred antigens are autoantigens.

In the specific embodiments described in the Examples, the antigen is the peptide p33 derived from lymphocytic choriomeningitis virus (LCMV). The p33 peptide represents one of the best studied CTL epitopes (Pircher et al., “Tolerance induction in double specific T-cell receptor transgenic mice varies with antigen,” Nature 342:559 (1989); Tissot et al., “Characterizing the functionality of recombinant T-cell receptors in vitro: a pMHC tetramer based approach,” J. Immunol Methods 236:147 (2000); Bachmann et al., “Four types of Ca2+-signals after stimulation of naive T cells with T cell agonists, partial agonists and antagonists,” Eur. J. Immunol. 27:3414 (1997); Bachmann et al., “Functional maturation of an anti-viral cytotoxic T cell response,” J. Virol. 71:5764 (1997); Bachmann et al., “Peptide induced TCR-down regulation on naive T cell predicts agonist/partial agonist properties and strictly correlates with T cell activation,” Eur. J. Immunol. 27:2195 (1997); Bachmann et al., “Distinct roles for LFA-1 and CD28 during activation of naive T cells: adhesion versus costimulation,” Immunity 7:549 (1997)). p33-specific T cells have been shown to induce lethal diabetic disease in transgenic mice (Ohashi et al., “Ablation of ‘tolerance’ and induction of diabetes by virus infection in viral antigen transgenic mice,” Cell 65:305 (1991)) as well as to be able to prevent growth of tumor cells expressing p33 (Kündig et al., “Fibroblasts act as efficient antigen-presenting cells in lymphoid organs,” Science 268:1343 (1995); Speiser et al., “CTL tumor therapy specific for an endogenous antigen does not cause autoimmune disease,” J. Exp. Med. 186:645 (1997)). This specific epitope, therefore, is particularly well suited to study autoimmunity, tumor immunology as well as viral diseases.

In one specific embodiment of the invention, the antigen or antigenic determinant is one that is useful for the prevention of infectious disease. Such treatment will be useful to treat a wide variety of infectious diseases affecting a wide range of hosts, e.g., human, cow, sheep, pig, dog, cat, other mammalian species and non-mammalian species as well. Treatable infectious diseases are well known to those skilled in the art, and examples include infections of viral etiology such as HIV, influenza, Herpes, viral hepatitis, Epstein Bar, polio, viral encephalitis, measles, chicken pox, Papilloma virus etc.; or infections of bacterial etiology such as pneumonia, tuberculosis, syphilis, etc.; or infections of parasitic etiology such as malaria, trypanosomiasis, leishmaniasis, trichomoniasis, amoebiasis, etc. Thus, antigens or antigenic determinants selected for the compositions of the invention will be well known to those in the medical art; examples of antigens or antigenic determinants include the following: the HIV antigens gp140 and gp160; the influenza antigens hemagglutinin, M2 protein and neuraminidase, Hepatitis B surface antigen or core and circumsporozoite protein of malaria or fragments thereof.

As discussed above, antigens include infectious microbes such as viruses, bacteria and fungi and fragments thereof, derived from natural sources or synthetically. Infectious viruses of both human and non-human vertebrates include retroviruses, RNA viruses and DNA viruses. The group of retroviruses includes both simple retroviruses and complex retroviruses. The simple retroviruses include the subgroups of B-type retroviruses, C-type retroviruses and D-type retroviruses. An example of a B-type retrovirus is mouse mammary tumor virus (MMTV). The C-type retroviruses include subgroups C-type group A (including Rous sarcoma virus (RSV), avian leukemia virus (ALV), and avian myeloblastosis virus (AMV)) and C-type group B (including murine leukemia virus (MLV), feline leukemia virus (FeLV), murine sarcoma virus (MSV), gibbon ape leukemia virus (GALV), spleen necrosis virus (SNV), reticuloendotheliosis virus (RV) and simian sarcoma virus (SSV)). The D-type retroviruses include Mason-Pfizer monkey virus (MPMV) and simian retrovirus type 1 (SRV-1). The complex retroviruses include the subgroups of lentiviruses, T-cell leukemia viruses and the foamy viruses. Lentiviruses include HIV-1, but also include HIV-2, SIV, Visna virus, feline immunodeficiency virus (FIV), and equine infectious anemia virus (EIAV). The T-cell leukemia viruses include HTLV-1, HTLV-II, simian T-cell leukemia virus (STLV), and bovine leukemia virus (BLV). The foamy viruses include human foamy virus (HFV), simian foamy virus (SFV) and bovine foamy virus (BFV).

Examples of RNA viruses that are antigens in vertebrate animals include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (ECHO) viruses, hepatitis A, C, D, E and G viruses, Simian enteroviruses, Murine encephalomyelitis (ME) viruses, Poliovirus muris, Bovine enteroviruses, Porcine enteroviruses, the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picornavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, Flanders-Hart Park virus), the genus Lyssavirus (Rabies virus), fish Rhabdoviruses, and filoviruses (Marburg virus and Ebola virus); the family Arenaviridae, including Lymphocytic choriomeningitis virus (LCM), Tacaribe virus complex, and Lassa virus; the family Coronoaviridae, including Infectious Bronchitis Virus (IBV), Mouse Hepatitis virus, Human enteric corona virus, and Feline infectious peritonitis (Feline coronavirus).

Illustrative DNA viruses that are antigens in vertebrate animals include, but are not limited to: the family Poxviridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesviruses (Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A, B, C, D and E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, Aleutian mink disease virus, etc.). Finally, DNA viruses may include viruses which do not fit into the above families such as Kuru and Creutzfeldt-Jacob disease viruses and chronic infectious neuropathic agents (CHINA virus).

Each of the foregoing lists is illustrative, and is not intended to be limiting.

In a specific embodiment of the invention, the antigen comprises one or more cytotoxic T cell epitopes, Th cell epitopes, or a combination of the two epitopes.

In addition to enhancing an antigen specific immune response in humans, the methods of the preferred embodiments are particularly well suited for treatment of other mammals or other animals, e.g., birds such as hens, chickens, turkeys, ducks, geese, quail and pheasant. Birds are prime targets for many types of infections.

An example of a common infection in chickens is chicken infectious anemia virus (CIAV). CIAV was first isolated in Japan in 1979 during an investigation of a Marek's disease vaccination break (Yuasa et al., Avian Dis. 23:366-385 (1979)). Since that time, CIAV has been detected in commercial poultry in all major poultry producing countries (van Bulow et al., pp. 690-699 in “Diseases of Poultry”, 9th edition, Iowa State University Press 1991).

Vaccination of birds, like other vertebrate animals can be performed at any age. Normally, vaccinations are performed at up to 12 weeks of age for a live microorganism and between 14-18 weeks for an inactivated microorganism or other type of vaccine. For in ovo vaccination, vaccination can be performed in the last quarter of embryo development. The vaccine can be administered subcutaneously, by spray, orally, intraocularly, intratracheally, nasally, in ovo or by other methods described herein.

Cattle and livestock are also susceptible to infection. Disease which affect these animals can produce severe economic losses, especially amongst cattle. The methods of the invention can be used to protect against infection in livestock, such as cows, horses, pigs, sheep and goats.

Cows can be infected by bovine viruses. Bovine viral diarrhea virus (BVDV) is a small enveloped positive-stranded RNA virus and is classified, along with hog cholera virus (HOCV) and sheep border disease virus (BDV), in the pestivirus genus. Although Pestiviruses were previously classified in the Togaviridae family, some studies have suggested their reclassification within the Flaviviridae family along with the flavivirus and hepatitis C virus (HCV) groups.

Equine herpesviruses (EHV) comprise a group of antigenically distinct biological agents which cause a variety of infections in horses ranging from subclinical to fatal disease. These include Equine herpesvirus-1 (EHV-1), a ubiquitous pathogen in horses. EHV-1 is associated with epidemics of abortion, respiratory tract disease, and central nervous system disorders. Other EHV's include EHV-2, or equine cytomegalovirus, EHV-3, equine coital exanthema virus, and EHV-4, previously classified as EHV-1 subtype 2.

Sheep and goats can be infected by a variety of dangerous microorganisms including visna-maedi.

Primates such as monkeys, apes and macaques can be infected by simian immunodeficiency virus. Inactivated cell-virus and cell-free whole simian immunodeficiency vaccines have been reported to afford protection in macaques (Stott et al., Lancet 36:1538-1541 (1990); Desrosiers et al., PNAS USA 86:6353-6357 (1989); Murphey-Corb et al., Science 246:1293-1297 (1989); and Carlson et al., AIDS Res. Human Retroviruses 6:1239-1246 (1990)). A recombinant HIV gp120 vaccine has been reported to afford protection in chimpanzees (Berman et al., Nature 345:622-625 (1990)).

Cats, both domestic and wild, are susceptible to infection with a variety of microorganisms. For instance, feline infectious peritonitis is a disease which occurs in both domestic and wild cats, such as lions, leopards, cheetahs, and jaguars. When it is desirable to prevent infection with this and other types of pathogenic organisms in cats, the methods of the invention can be used to vaccinate cats to prevent them against infection.

Domestic cats may become infected with several retroviruses, including but not limited to feline leukemia virus (FeLV), feline sarcoma virus (FeSV), endogenous type C oncomavirus (RD-114), and feline syncytia-forming virus (FeSFV). The discovery of feline T-lymphotropic lentivirus (also referred to as feline immunodeficiency) was first reported in Pedersen et al., Science 235:790-793 (1987). Feline infectious peritonitis (FIP) is a sporadic disease occurring unpredictably in domestic and wild Felidae. While FIP is primarily a disease of domestic cats, it has been diagnosed in lions, mountain lions, leopards, cheetahs, and the jaguar. Smaller wild cats that have been afflicted with FIP include the lynx and caracal, sand cat and pallas cat.

Viral and bacterial diseases in fin-fish, shellfish or other aquatic life forms pose a serious problem for the aquaculture industry. Owing to the high density of animals in the hatchery tanks or enclosed marine farming areas, infectious diseases may eradicate a large proportion of the stock in, for example, a fin-fish, shellfish, or other aquatic life forms facility. Prevention of disease is a more desired remedy to these threats to fish than intervention once the disease is in progress. Vaccination of fish is the only preventative method which may offer long-term protection through immunity. Nucleic acid based vaccinations of fish are described, for example, in U.S. Pat. No. 5,780,448.

The fish immune system has many features similar to the mammalian immune system, such as the presence of B cells, T cells, lymphokines, complement, and immunoglobulins. Fish have lymphocyte subclasses with roles that appear similar in many respects to those of the B and T cells of mammals. Vaccines can be administered orally or by immersion or injection.

Aquaculture species include but are not limited to fin-fish, shellfish, and other aquatic animals. Fin-fish include all vertebrate fish, which may be bony or cartilaginous fish, such as, for example, salmonids, carp, catfish, yellowtail, seabream and seabass. Salmonids are a family of fin-fish which include trout (including rainbow trout), salmon and Arctic char. Examples of shellfish include, but are not limited to, clams, lobster, shrimp, crab and oysters. Other cultured aquatic animals include, but are not limited to, eels, squid and octopi.

Polypeptides of viral aquaculture pathogens include but are not limited to glycoprotein or nucleoprotein of viral hemorrhagic septicemia virus (VHSV); G or N proteins of infectious hematopoietic necrosis virus (IHNV); VP1, VP2, VP3 or N structural proteins of infectious pancreatic necrosis virus (IPNV); G protein of spring viremia of carp (SVC); and a membrane-associated protein, tegumin or capsid protein or glycoprotein of channel catfish virus (CCV).

Polypeptides of bacterial pathogens include but are not limited to an iron-regulated outer membrane protein, (IROMP), an outer membrane protein (OMP), and an A-protein of Aeromonis salmonicida which causes furunculosis, p57 protein of Renibacterium salmoninarum which causes bacterial kidney disease (BKD), major surface associated antigen (msa), a surface expressed cytotoxin (mpr), a surface expressed hemolysin (ish), and a flagellar antigen of Yersiniosis; an extracellular protein (ECP), an iron-regulated outer membrane protein (IROMP), and a structural protein of Pasteurellosis; an OMP and a flagellar protein of Vibrosis anguillarum and V. ordalii; a flagellar protein, an OMP protein, aroA, and purA of Edwardsiellosis ictaluri and E. tarda; and surface antigen of Ichthyophthirius; and a structural and regulatory protein of Cytophaga columnari; and a structural and regulatory protein of Rickettsia.

Polypeptides of a parasitic pathogen include but are not limited to the surface antigens of Ichthyophthirius.

In another aspect of the invention, there is provided vaccine compositions suitable for use in methods for preventing and/or attenuating diseases or conditions which are caused or exacerbated by “self” gene products (e.g., tumor necrosis factors). Thus, vaccine compositions of the invention include compositions which lead to the production of antibodies that prevent and/or attenuate diseases or conditions caused or exacerbated by “self” gene products. Examples of such diseases or conditions include graft versus host disease, IgE-mediated allergic reactions, anaphylaxis, adult respiratory distress syndrome, Crohn's disease, allergic asthma, acute lymphoblastic leukemia (ALL), non-Hodgkin's lymphoma (NHL), Graves' disease, systemic lupus erythematosus (SLE), inflammatory autoimmune diseases, myasthenia gravis, immunoproliferative disease lymphadenopathy (IPL), angioimmunoproliferative lymphadenopathy (AIL), immunoblastive lymphadenopathy (IBL), rheumatoid arthritis, diabetes, multiple sclerosis, Alzheimer disease and osteoporosis.

In related specific embodiments, compositions of the invention are an immunotherapeutic that can be used for the treatment and/or prevention of allergies, cancer or drug addiction.

The selection of antigens or antigenic determinants for the preparation of compositions and for use in methods of treatment for allergies would be known to those skilled in the medical arts treating such disorders. Representative examples of such antigens or antigenic determinants include the following: bee venom phospholipase A₂, Bet v I (birch pollen allergen), 5 Dol m V (white-faced hornet venom allergen), and Der p I (House dust mite allergen), as well as fragments of each which can be used to elicit immunological responses.

The selection of antigens or antigenic determinants for compositions and methods of treatment for cancer would be known to those skilled in the medical arts treating such disorders (see Renkvist et al., Cancer Immunol. Immunother. 50:3-15 (2001) which is incorporated by reference), and such antigens or antigenic determinants are included within the scope of the present invention. Representative examples of such types of antigens or antigenic determinants include the following: Her2 (breast cancer); GD2 (neuroblastoma); EGF-R (malignant glioblastoma); CEA (medullary thyroid cancer); CD52 (leukemia); human melanoma protein gp100; human melanoma protein gp100 epitopes such as amino acids 154-162 (sequence: KTWGQYWQV), 209-217 (ITDQVPFSV), 280-288 (YLEPGPVTA), 457-466 (LLDGTATLRL) and 476-485 (VLYRYGSFSV); human melanoma protein melan-A/MART-1; human melanoma protein melan-A/MART-1 epitopes such as amino acids 27-35 (AAGIGILTV) and 32-40 (ILTVILGVL); tyrosinase; tyrosinase epitopes such as amino acids 1-9 (MLLAVLYCL) and 368-376 (YMDGTMSQV); NA17-A nt protein; NA17-A nt protein epitopes such as amino acids 38-64 (VLPDVFIRC); MAGE-3 protein; MAGE-3 protein epitopes such as amino acids 271-279 (FLWGPRALV); other human tumors antigens, e.g. CEA epitopes such as amino acids 571-579 (YLSGANLNL); p53 protein; p53 protein epitopes such as amino acids 65-73 (RMPEAAPPV), 149-157 (STPPPGTRV) and 264-272 (LLGRNSFEV); Her2/neu epitopes such as amino acids 369-377 (KIFGSLAFL) and 654-662 (IISAVVGIL); HPV16 E7 protein; HPV16 E7 protein epitopes such as amino acids 86-93 (TLGIVCPI); as well as fragments of each which can be used to elicit immunological responses.

The selection of antigens or antigenic determinants for compositions and methods of treatment for drug addiction, in particular recreational drug addiction, would be known to those skilled in the medical arts treating such disorders. Representative examples of such antigens or antigenic determinants include, for example, opioids and morphine derivatives such as codeine, fentanyl, heroin, morphium and opium; stimulants such as amphetamine, cocaine, MDMA (methylenedioxymethamphetamine), methamphetamine, methylphenidate and nicotine; hallucinogens such as LSD, mescaline and psilocybin; as well as cannabinoids such as hashish and marijuana.

The selection of antigens or antigenic determinants for compositions and methods of treatment for other diseases or conditions associated with self antigens would be also known to those skilled in the medical arts treating such disorders. Representative examples of such antigens or antigenic determinants are, for example, lymphotoxins (e.g. Lymphotoxin α (LT α), Lymphotoxin β (LT β)), and lymphotoxin receptors, Receptor activator of nuclear factor kappaB ligand (RANKL), vascular endothelial growth factor (VEGF) and vascular endothelial growth factor receptor (VEGF-R), Interleukin 17 and amyloid beta peptide (Aβ₁₋₄₂), TNFα, MIF, MCP-1, SDF-1, Rank-L, M-CSF, Angiotensin II, Endoglin, Eotaxin, BLC, CCL21, IL-13, IL-17, IL-5, Bradykinin, Resistin, LHRH, GHRH, GIH, CRH, TRH and Gastrin, as well as fragments of each which can be used to elicit immunological responses.

In a particular embodiment of the invention, the antigen or antigenic determinant is selected from the group consisting of: (a) a recombinant polypeptide of HIV; (b) a recombinant polypeptide of Influenza virus (e.g., an Influenza virus M2 polypeptide or a fragment thereof); (c) a recombinant polypeptide of Hepatitis C virus; (d) a recombinant polypeptide of Hepatitis B virus; (e) a recombinant polypeptide of Toxoplasma; (f) a recombinant polypeptide of Plasmodium falciparum; (g) a recombinant polypeptide of Plasmodium vivax; (h) a recombinant polypeptide of Plasmodium ovale; (i) a recombinant polypeptide of Plasmodium malariae; (j) a recombinant polypeptide of breast cancer cells; (k) a recombinant polypeptide of kidney cancer cells; (l) a recombinant polypeptide of prostate cancer cells; (m) a recombinant polypeptide of skin cancer cells; (n) a recombinant polypeptide of brain cancer cells; (o) a recombinant polypeptide of leukemia cells; (p) a recombinant profiling; (q) a recombinant polypeptide of bee sting allergy; (r) a recombinant polypeptide of nut allergy; (s) a recombinant polypeptide of pollen; (t) a recombinant polypeptide of house-dust; (u) a recombinant polypeptide of cat or cat hair allergy; (v) a recombinant protein of food allergies; (w) a recombinant protein of asthma; (x) a recombinant protein of Chlamydia; and (y) a fragment of any of the polypeptides set out in (a)-(x).

In another embodiment of the present invention, the antigen, being coupled, fused or otherwise attached to the virus-like particle, is a T cell epitope, either a cytotoxic or a Th cell epitope. In a further preferred embodiment, the antigen is a combination of at least two, preferably different, epitopes, wherein the at least two epitopes are linked directly or by way of a linking sequence. These epitopes are preferably selected from the group consisting of cytotoxic and Th cell epitopes.

It should also be understood that a mosaic virus-like particle, e.g. a virus-like particle composed of subunits attached to different antigens and epitopes, respectively, is within the scope of the present invention. Such a composition of the present invention can be, for example, obtained by transforming E. coli with two compatible plasmids encoding the subunits composing the virus-like particle fused to different antigens and epitopes, respectively. In this instance, the mosaic virus-like particle is assembled either directly in the cell or after cell lysis. Moreover, such an inventive composition can also be obtained by attaching a mixture of different antigens and epitopes, respectively, to the isolated virus-like particle.

The antigen of the present invention, and in particular the indicated epitope or epitopes, can be synthesized or recombinantly expressed and coupled to the virus-like particle, or fused to the virus-like particle using recombinant DNA techniques. Exemplary procedures describing the attachment of antigens to virus-like particles are disclosed in WO 00/32227.

Another element in the composition of the invention is a substance that activates antigen presenting cells in an amount sufficient to enhance the immune response of an animal to an antigen.

The invention relates to the surprising and unexpected finding that stimulation of antigen presenting cell (APC) activation dramatically enhances the specific T cell response obtained after vaccination with virus like particles coupled, fused or otherwise attached to antigens. For example, while vaccination with recombinant VLPs containing a cytotoxic T cell (CTL) epitope of lymphocytic choriomeningitis virus induced low levels cytolytic activity and did not induce efficient anti-viral protection, VLPs fused to the viral CTL epitope injected together with anti-CD40 antibodies or CpGs induced strong CTL activity and full anti-viral protection (Examples 3, 4, 6 and 7).

Also unexpectedly, stimulation of innate immunity was more efficient at enhancing CTL responses induced by VLPs fused or coupled to an antigen than CTL responses induced by free peptide (Examples 5, 15 and 16). The technology allows the creation of highly efficient vaccines against infectious diseases and for the creation of vaccines for the treatment of cancers.

In general, any substance that activates antigen presenting cells can be used within the scope of the present invention, provided that the addition of the substance enhances an immune response of an animal, e.g. human, to a desired antigen. In addition, the substance can stimulate any activity associated with antigen presenting cells known by those of skill in the art. For example, the substance can stimulate upregulation of costimulatory molecules on or cytokine production in antigen presenting cells, and/or induce nuclear translocation of NFκB in antigen presenting cells and/or activate toll-like receptors in antigen presenting cells to enhance the immune response against an antigen.

In a specific embodiment, the substance comprises, or alternatively consists of, an immunostimulatory nucleic acid, in particular an unmethylated CpG-containing oligonucleotide (CpGs) or compounds that activate CD40, such as anti-CD40 antibodies.

The anti-CD40 antibodies of the invention can be produced by any suitable method known in the art for the synthesis of antibodies, in particular, by chemical synthesis or preferably, by recombinant expression techniques. (See, e.g. U.S. Pat. Nos. 6,056,959; 6,051,228; and 5,801,227.)

Polyclonal antibodies to an antigen-of-interest can be produced by various procedures well known in the art. For example, a CD40 polypeptide can be administered to various host animals including, but not limited to, rabbits, mice, rats, etc. to induce the production of sera containing polyclonal antibodies specific for the antigen. Various adjuvants may be used to increase the immunological response depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal antibodies can be prepared using a wide variety of techniques known in the art including the use of hybridoma, recombinant and phage display technologies, or a combination thereof. For example, monoclonal antibodies can be produced using hybridoma techniques including those known in the art and taught, for example, in Harlow et al., “Antibodies: A Laboratory Manual,” (Cold Spring Harbor Laboratory Press, 2nd ed. 1988); Hammerling et al., in: “Monoclonal Antibodies and T-Cell Hybridomas” 563-681 (Elsevier, N.Y., 1981) (said references incorporated by reference in their entireties). The term “monoclonal antibody” is not limited to antibodies produced through hybridoma technology. The term “monoclonal antibody” refers to an antibody that is derived from a single clone, including any eukaryotic, prokaryotic, or phage clone, and not the method by which it is produced.

Alternatively, antibodies of the present invention can be produced through the application of recombinant DNA and phage display technology or through synthetic chemistry using methods known in the art. For example, the antibodies of the present invention can be prepared using various phage display methods known in the art. In phage display methods, functional antibody domains are displayed on the surface of a phage particle which carries polynucleotide sequences encoding them. Phage with a desired binding property are selected from a repertoire or combinatorial antibody library (e.g. human or murine) by selecting directly with antigen, typically antigen bound or captured to a solid surface or bead. Phage used in these methods are typically filamentous phage including fd and M13 with Fab, Fv or disulfide stabilized Fv antibody domains recombinantly fused to preferably the phage gene III or alternatively gene VIII protein. Examples of phage display methods that can be used to make the antibodies of the present invention include those disclosed in Brinkman U. et al., J. Immunol. Methods 182:41-50 (1995); Ames, R. S. et al., J. Immunol. Methods 184:177-186 (1995); Kettleborough, C. A. et al., Eur. J. Immunol. 24:952-958 (1994); Persic, L. et al., Gene 187:9-18 (1997); Burton, D. R. et al., Advances in Immunology 57:191-280 (1994); PCT/GB91/01134; WO 90/02809; WO 91/10737; WO 92/01047; WO 92/18619; WO 93/11236; WO 95/15982; WO 95/20401; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743 (said references incorporated by reference in their entireties).

As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast and bacteria. For example, techniques to recombinantly produce Fab, Fab′ and F(ab′)2 fragments can also be employed using methods known in the art such as those disclosed in WO 92/22324; Mullinax, R. L. et al., BioTechniques 12:864-869 (1992); and Sawai, H. et al. AJRI 34:26-34 (1995); and Better, M. et al., Science 240:1041-1043 (1988) (said references incorporated by reference in their entireties).

Examples of techniques which can be used to produce single-chain Fvs and antibodies include those described in U.S. Pat. Nos. 4,946,778 and 5,258,498; Huston et al., Methods in Enzymology 203:46-88 (1991); Shu, L. et al., PNAS 90:7995-7999 (1993); and Skerra, A. et al., Science 240:1038-1040 (1988).

For some uses, including in vivo use of antibodies in humans, it may be preferable to use chimeric, humanized, or human antibodies. Methods for producing chimeric antibodies are known in the art. See e.g., Morrison, Science 229:1202 (1985); Oi et al., BioTechniques 4:214 (1986); Gillies, S. D. et al., J. Immunol. Methods 125:191-202 (1989); and U.S. Pat. No. 5,807,715. Antibodies can be humanized using a variety of techniques including CDR-grafting (EP 0 239 400; WO 91/09967; U.S. Pat. Nos. 5,530,101; and 5,585,089), veneering or resurfacing (EP 0 592 106; EP 0 519 596; Padlan E. A., Molecular Immunology 28(4/5):489-498 (1991); Studnicka G. M. et al., Protein Engineering 7:805-814 (1994); Roguska M. A. et al., PNAS 91:969-973 (1994)), and chain shuffling (U.S. Pat. No. 5,565,332). Human antibodies can be made by a variety of methods known in the art including phage display methods described above. See also, U.S. Pat. Nos. 4,444,887, 4,716,111, 5,545,806, and 5,814,318; and WO 98/46645, WO 98/50433, WO 98/24893, WO 98/16654, WO 96/34096, WO 96/33735 and WO 91/10741 (said references incorporated by reference in their entireties).

In a specific aspect of the invention, immunostimulatory nucleic acids, in particular unmethylated CpG-containing oligonucleotides are used to induce activation of immune cells and preferably professional APCs. As used herein, professional APC has its ordinary meaning in the art and includes, for instance, monocytes/macrophages and in particular dendritic cells such as immature dendritic cells and precursor and progenitor dendritic cells, as well as mature dendritic cells which are capable of taking up and presenting antigen. Such a population of APC or dendritic cells is referred to as a primed population of APCs or dendritic cells.

The innate immune system has the capacity to recognize invariant molecular pattern shared by microbial pathogens. Recent studies have revealed that this recognition is a crucial step in inducing effective immune responses. The main mechanism by which microbial products augment immune responses is to stimulate APC, especially dendritic cells to produce proinflammatory cytokines and to express high levels costimulatory molecules for T cells. These activated dendritic cells subsequently initiate primary T cell responses and dictate the type of T cell-mediated effector function.

Two classes of nucleic acids, namely 1) bacterial DNA that contains immunostimulatory sequences, in particular unmethylated CpG dinucleotides within specific flanking bases (referred to as CpG motifs) and 2) double-stranded RNA synthesized by various types of viruses represent important members of the microbial components that enhance immune responses. Synthetic double stranded (ds) RNA such as polyinosinic-polycytidylic acid (poly I:C) are capable of inducing dendritic cells to produce proinflammatory cytokines and to express high levels of costimulatory molecules.

A series of studies by Tokunaga and Yamamoto et al. has shown that bacterial DNA or synthetic oligodeoxynucleotides induce human PBMC and mouse spleen cells to produce type I interferon (IFN) (reviewed in Yamamoto et al., Springer Semin Immunopathol. 22:11-19). Poly (I:C) was originally synthesized as a potent inducer of type I IFN but also induces other cytokines such as IL-12.

Preferred ribonucleic acid encompass polyinosinic-polycytidylic acid double-stranded RNA (poly I:C). Ribonucleic acids and modifications thereof as well as methods for their production have been described by Levy, H. B (Methods Enzymol. 78:242-251 (1981)), DeClercq, E (Methods Enzymol. 78:227-236 (1981)) and Torrence, P. F. (Methods Enzymol 78:326-331 (1981)) and references therein. Ribonucleic acids can be isolated from organisms. Ribonucleic acids also encompass further synthetic ribonucleic acids, in particular synthetic poly (I:C) oligonucleotides that have been rendered nuclease resistant by modification of the phosphodiester backbone, in particular by phosphorothioate modifications. In a further embodiment the ribose backbone of poly (I:C) is replaced by a deoxyribose. Those skilled in the art know procedures how to synthesize synthetic oligonucleotides.

In another preferred embodiment of the invention molecules that active toll-like receptors (TLR) are enclosed. Ten human toll-like receptors are known uptodate. They are activated by a variety of ligands. TLR2 is activated by peptidoglycans, lipoproteins, lipoteichonic acid and Zymosan; TLR3 is activated by double-stranded RNA such as poly (I:C); TLR4 is activated by lipopolysaccharide, lipoteichoic acids and taxol; TLR5 is activated by bacterial flagella, especially the flagellin protein; TLR6 is activated by peptidoglycans, TLR7 is activated by imiquimoid and imidazoquinoline compounds, such as R418 and TLR9 is activated by bacterial DNA, in particular CpG DNA. Ligands for TLR1, TLR8 and TLR10 are not known so far. However, recent reports indicate that same receptors can react with different ligands and that further receptors are present. The above list of ligands is not exhaustive and further ligands are within the knowledge of the person skilled in the art.

In general, the unmethylated CpG-containing oligonucleotide comprises the sequence:

5′ X₁X₂CGX₃X₄ 3′

wherein X₁, X₂, X₃ and X₄ are any nucleotide. In addition, the oligonucleotide can comprise about 6 to about 100,000 nucleotides, preferably about 6 to about 2000 nucleotides, more preferably about 20 to about 2000 nucleotides, and even more preferably comprises about 20 to about 300 nucleotides.

In a preferred embodiment, the CpG oligonucleotide contains one or more phosphorothioate modifications of the phosphate backbone. For example, a CpG-containing oligonucleotide having one or more phosphate backbone modifications or having all of the phosphate backbone modified and wherein one, some or all of the nucleotide phosphate backbone modifications are phosphorothioate modifications is included within the scope of the present invention. Further methods to modify the oligonucleotide backbone are in the knowledge of those skilled in the art.

The CpG-containing oligonucleotide can also be recombinant, genomic, synthetic, cDNA, plasmid-derived and single or double stranded. For use in the instant invention, the nucleic acids can be synthesized de novo using any of a number of procedures well known in the art. For example, the b-cyanoethyl phosphoramidite method (Beaucage, S. L., and Caruthers, M. H., Tet. Let. 22:1859 (1981); nucleoside H-phosphonate method (Garegg et al., Tet. Let. 27:4051-4054 (1986); Froehler et al., Nucl. Acid. Res. 14:5399-5407 (1986); Garegg et al., Tet. Let. 27:4055-4058 (1986), Gaffney et al., Tet. Let. 29:2619-2622 (1988)). These chemistries can be performed by a variety of automated oligonucleotide synthesizers available in the market. Alternatively, CpGs can be produced on a large scale in plasmids, (see Sambrook, T., et al., “Molecular Cloning: A Laboratory Manual,” Cold Spring Harbor laboratory Press, New York, 1989) which after being administered to a subject are degraded into oligonucleotides. Oligonucleotides can be prepared from existing nucleic acid sequences (e.g., genomic or cDNA) using known techniques, such as those employing restriction enzymes, exonucleases or endonucleases.

In yet another specific embodiment, the antigen presenting cells are dendritic cells. Dendritic cells form the link between the innate and the acquired immune system by presenting antigens as well as through their expression of pattern recognition receptors which detect microbial molecules in their local environment. Dendritic cells efficiently internalize, process, and present soluble and particulate antigen to which it is exposed. If the DC is activated during or after internalization by, for example, CpGs, upregulation of the expression of major histocompatibility complex (MHC) and costimulatory molecules rapidly occurs and the production of cytokines including IL-12 or interferon α is induced followed by migration toward lymphatic organs where they are believed to be involved in the activation of T cells.

Dendritic cells useful according to the invention can be isolated from any source as long as the cell is capable of being activated by substances such as anti-CD40 antibodies and immunostimulatory nucleic acids, in particular CpGs to produce an active antigen expressing dendritic cell. Sources can easily be determined by those of skill in the art without requiring undue experimentation, by for instance, isolating a primary source of dendritic cells and testing activation by anti-CD40 antibodies and/or immunostimulatory nucleic acids, in particular CpGs in vitro.

One specific use for the anti-CD40 antibodies and/or immunostimulatory nucleic acids, in particular CpG oligomers of the invention is to activate dendritic cells for the purpose of enhancing a specific immune response against antigens. The immune response can be enhanced using ex vivo or in vivo techniques. The ex vivo procedure can be used on autologous or heterologous cells, but is preferably used on autologous cells. In preferred embodiments, the dendritic cells are isolated from peripheral blood or bone marrow, but can be isolated from any source of dendritic cells. When the ex vivo procedure is performed to specifically produce dendritic cells active against a specific cancer or other type of antigen, the dendritic cells can be exposed to the antigen in addition to the anti-CD40 antibodies and/or immunostimulatary nucleic acids, in particular CpGs. In other cases the dendritic cell can have already been exposed to antigen but may not be displaying epitopes of the antigen on the surface efficiently. Alternatively the dendritic cell may be exposed to the antigen, by either direct contact or exposure in the body and then the dendritic cell is returned to the body followed by administration of anti-CD40 antibodies and/or immunostimulatory nucleic acids, in particular CpGs directly to the subject, either systemically or locally.

When returned to the subject, the activated dendritic cell expressing the antigen activates T cells in vivo which are specific for the antigen. Ex vivo manipulation of dendritic cells for the purposes of cancer immunotherapy have been described in several references in the art, including Engleman, E. G., Cytotechnology 25:1 (1997); Van Schooten, W., et al., Molecular Medicine Today, June, 255 (1997); Steinman, R. M., Experimental Hematology 24:849 (1996); and Gluckman, J. C., Cytokines, Cellular and Molecular Therapy 3:187 (1997).

The dendritic cells can also be contacted with anti-CD40 antibodies and/or immunostimulatory nucleic acids, in particular CpGs using in vivo methods. In order to accomplish this, anti-CD40 antibodies and/or immunostimulatory nucleic acids, in particular CpGs are administered directly to a subject in need of immunotherapy. The anti-CD40 antibodies and/or immunostimulatory nucleic acids, in particular CpGs can be administered in combination with the VLP coupled, fused or otherwise attached to an antigen or can be administered alone either before or after administration of the VLP coupled, fused or otherwise attached to an antigen. In some embodiments, it is preferred that the anti-CD40 antibodies and/or immunostimulatory nucleic acids, in particular CpGs be administered in the local region of the tumor, which can be accomplished in any way known in the art, e.g., direct injection into the tumor.

In yet another embodiment, the APCs activated by the immunostimulatory nucleic acids, in particular CpGs are NK or B cells. NK cells and B cells produce cytokines including interferons upon stimulation with certain types of CpGs which leads to enhanced T cell responses, in particular in humans.

The invention also provides vaccine compositions which can be used for preventing and/or attenuating diseases or conditions. Vaccine compositions of the invention comprise, or alternatively consist of, an immunologically effective amount of the inventive immune enhancing composition together with a pharmaceutically acceptable diluent, carrier or excipient. The vaccine can also optionally comprise an adjuvant.

The invention further provides vaccination methods for preventing and/or attenuating diseases or conditions in animals. Also provided are methods of enhancing anti-viral protection in an animal.

In one embodiment, the invention provides vaccines for the prevention of infectious diseases in a wide range of animal species, particularly mammalian species such as human, monkey, cow, dog, cat, horse, pig, etc. Vaccines can be designed to treat infections of viral etiology such as HIV, influenza, Herpes, viral hepatitis, Epstein Bar, polio, viral encephalitis, measles, chicken pox, etc.; or infections of bacterial etiology such as pneumonia, tuberculosis, syphilis, etc.; or infections of parasitic etiology such as malaria, trypanosomiasis, leishmaniasis, trichomoniasis, amoebiasis, etc.

In another embodiment, the invention provides vaccines for the prevention of cancer in a wide range of species, particularly mammalian species such as human, monkey, cow, dog, cat, horse, pig, etc. Vaccines can be designed to treat all types of cancer including, but not limited to, lymphomas, carcinomas, sarcomas and melanomas.

In another embodiment, the invention provides vaccines suited to boost existing T cell responses. In yet another embodiment, the invention provides vaccines that prime T cell responses that may be boosted by homologous or heterologous T cell responses.

As would be understood by one of ordinary skill in the art, when compositions of the invention are administered to an animal, they can be in a composition which contains salts, buffers, adjuvants or other substances which are desirable for improving the efficacy of the composition. Examples of materials suitable for use in preparing pharmaceutical compositions are provided in numerous sources including REMINGTON'S PHARMACEUTICAL SCIENCES (Osol, A, ed., Mack Publishing Co., (1990)).

Various adjuvants can be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art. Further adjuvants that can be administered with the compositions of the invention include, but are not limited to, Monophosphoryl lipid immunomodulator, AdjuVax 100a, QS-21, QS-18, CRL1005, Aluminum salts, MF-59, and Virosomal adjuvant technology. The adjuvants can also comprise a mixture of these substances.

Compositions of the invention are said to be “pharmacologically acceptable” if their administration can be tolerated by a recipient individual. Further, the compositions of the invention will be administered in a “therapeutically effective amount” (i.e., an amount that produces a desired physiological effect).

The compositions of the present invention can be administered by various methods known in the art. The particular mode selected will depend of course, upon the particular composition selected, the severity of the condition being treated and the dosage required for therapeutic efficacy. The methods of the invention, generally speaking, can be practiced using any mode of administration that is medically acceptable, meaning any mode that produces effective levels of the active compounds without causing clinically unacceptable adverse effects. Such modes of administration include oral, rectal, parenteral, intracistemal, intravaginal, intraperitoneal, topical (as by powders, ointments, drops or transdermal patch), bucal, or as an oral or nasal spray. The term “parenteral” as used herein refers to modes of administration which include intravenous, intramuscular, intraperitoneal, intrasternal, subcutaneous and intraarticular injection and infusion. The composition of the invention can also be injected directly in a lymph node.

Components of compositions for administration include sterile aqueous (e.g., physiological saline) or non-aqueous solutions and suspensions. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption.

Combinations can be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

Dosage levels depend on the mode of administration, the nature of the subject, and the quality of the carrier/adjuvant formulation. Typical amounts are in the range of about 0.1 μg to about 20 mg per subject. Preferred amounts are at least about 1 μg to about 100 μg per subject. Multiple administration to immunize the subject is preferred, and protocols are those standard in the art adapted to the subject in question.

The compositions can conveniently be presented in unit dosage form and can be prepared by any of the methods well-known in the art of pharmacy. Methods include the step of bringing the compositions of the invention into association with a carrier which constitutes one or more accessory ingredients. In general, the compositions are prepared by uniformly and intimately bringing the compositions of the invention into association with a liquid carrier, a finely divided solid carrier, or both, and then, if necessary, shaping the product.

Compositions suitable for oral administration can be presented as discrete units, such as capsules, tablets, lozenges, each containing a predetermined amount of the compositions of the invention. Other compositions include suspensions in aqueous liquids or non-aqueous liquids such as a syrup, elixir or an emulsion.

Other delivery systems can include time-release, delayed release or sustained release delivery systems. Such systems can avoid repeated administrations of the compositions of the invention described above, increasing convenience to the subject and the physician. Many types of release delivery systems are available and known to those of ordinary skill in the art.

Other embodiments of the invention include processes for the production of the compositions of the invention and methods of medical treatment for cancer and allergies using said compositions.

The following examples are illustrative only and are not intended to limit the scope of the invention as defined by the appended claims. It will be apparent to those skilled in the art that various modifications and variations can be made in the methods of the present invention without departing from the spirit and scope of the invention. Thus, it is intended that the present invention cover the modifications and variations of this invention provided they come within the scope of the appended claims and their equivalents.

All patents and publications referred to herein are expressly incorporated by reference in their entirety.

TABLE II
Sequences of immunostimulatory nucleic acids
used in the Examples.
Small letters indicate deoxynucleotides connected
via phosphorothioate bonds.

| | |
|---|---|
| CyCpGpt | tccatgacgttcctgaataat |
| B-CpGpt | tccatgacgttcctgacgtt |
| NKCpGpt | ggggtcaacgttgaggggg |
| CyCpG-rev-pt | attattcaggaacgtcatgga |
| G10pt | gggggggggggacgatcgtcgggggggggg |
| CyOpApt | tccatgacgttcctgaataataaatgcatgtcaaaga |
| | cagcat |
| CyCyCypt | tccatgacgttcctgaataattccatgacgttcctga |
| | ataattccatgacgttcctgaataat |
| CyCpG(20)pt | tccatgacgttcctgaataatcgcgcgcgcgcgcgcg |
| | cgcgcgcgcgcgcgcgcgcgcgcg |
| 2006pt | tcgtcgttttgtcgttttgtcgt |
| 5126PS | ggttcttttggtccttgtct |

### Example 1

### Generation of p33-VLPs

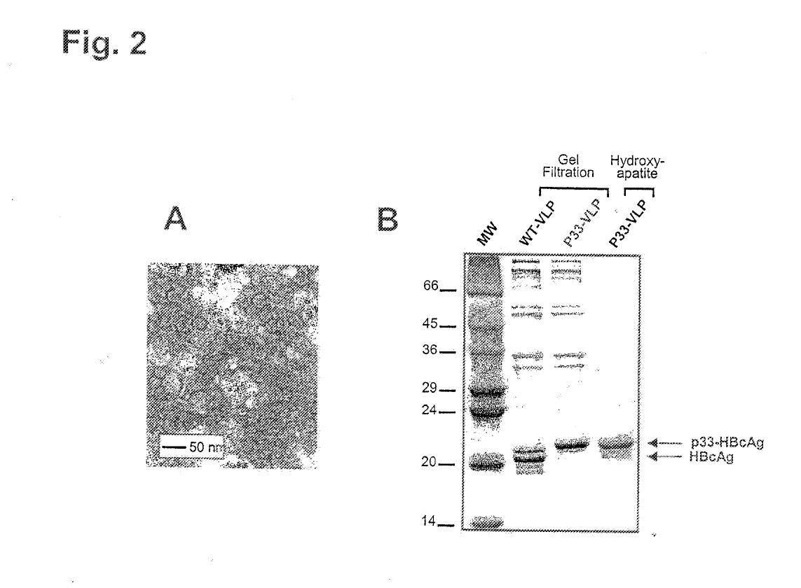

The DNA sequence of HBcAg containing peptide p33 from LCMV is given in FIG. 1. The p33-VLPs were generated as follows: Hepatitis B clone pEco63 containing the complete viral genome of Hepatitis B virus was purchased from ATCC. The gene encoding HBcAg was introduced into the EcoRI/HindIII restriction sites of expression vector pkk223.3 (Pharmacia) under the control of a strong tac promoter. The p33 peptide (KAVYNFATM) derived from lymphocytic choriomeningitis virus (LCMV) was fused to the C-terminus of HBcAg (1-183) via a three leucine-linker by standard PCR methods. A clone of E. coli K802 selected for good expression was transfected with the plasmid, and cells were grown and resuspended in 5 ml lysis buffer (10 mM Na₂HPO₄, 30 mM NaCl, 10 mM EDTA, 0.25% Tween-20, pH 7.0). 200 μl of lysozyme solution (20 mg/ml) was added. After sonication, 4 μl Benzonase and 10 mM MgCl₂ was added and the suspension was incubation for 30 minutes at RT, centrifuged for 15 minutes at 15,000 rpm at 4° C. and the supernatant was retained. Next, 20% (w/v) (0.2 g/ml lysate) ammonium sulfate was added to the supernatant. After incubation for 30 minutes on ice and centrifugation for 15 minutes at 20,000 rpm at 4° C. the supernatant was discarded and the pellet resuspended in 2-3 ml PBS. 20 ml of the PBS-solution was loaded onto a Sephacryl S-400 gel filtration column (Amersham Pharmacia Biotechnology AG), fractions were loaded onto a SDS-Page gel and fractions with purified HBc capsids were pooled. Pooled fractions were loaded onto a Hydroxyappatite column. Flow through (which contains purified HBc capsids) was collected. Electron microscopy was performed according to standard protocols. A representative example is shown in FIG. 2.

### Example 2

### P33-VLPs are Efficiently Processed by DCs and Macrophages

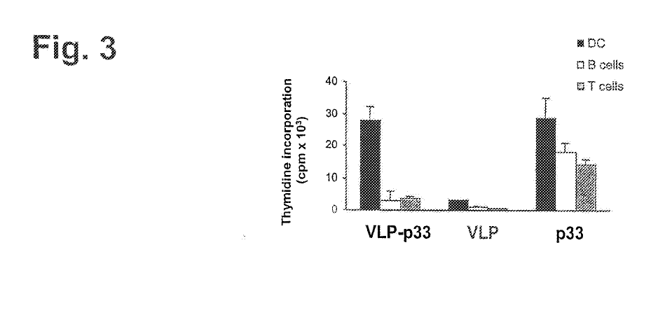

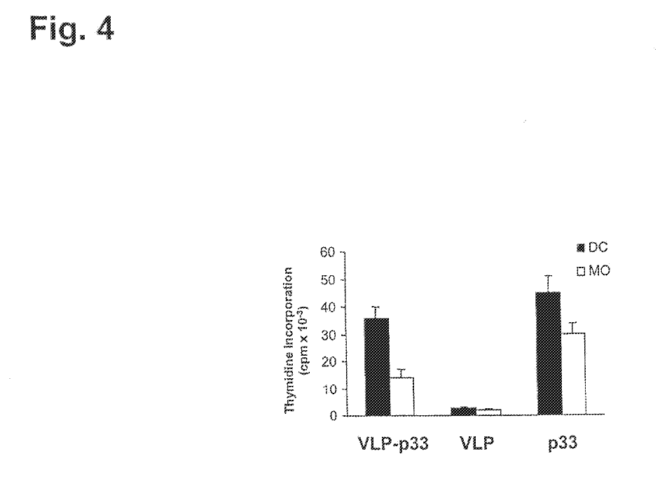

DCs were isolated from lymphoid organs as described (Ruedl, C., et al., Eur. J. Immunol. 26:1801 (1996)). Briefly, organs were collected and digested twice for 30 min at 37° C. in IMDM supplemented with 5% FCS and 100 μg/ml Collagenase D (Boehringer Mannheim, Mannheim, Germany). Released cells were recovered and resuspended in an Optiprep-gradient (Nycomed, Norway) and centrifuged at 600×g for 15 min. Low-density cells in the interfase were collected and stained with an anti-CD11c antibody. DCs were purified by sorting with a FACSStar^{plus} (Becton Dickinson, Mountain view, CA) on the basis of CD11c expression and excluding propidium iodide positive cells. Purified DCs, B and T cells (FIG. 3) obtained from spleens and thioglycollate-stimulated peritoneal macrophages (FIG. 4) were pulsed for 1 h with various concentrations of p33-VLP, VLP (1-0.01 μg/ml) or the peptide p33 (10-0.100 ng/ml). After three washings, presenter cells were co-cultured together with antigen-specific transgenic CD8⁺ T cells. After two days, T cell proliferation was measured by ³[H]thymidine uptake in a 16-h pulse (1 μCi/well).

### Example 3

### P33-VLPs Injected with Anti-CD40 Antibodies Induce Enhanced CTL Activity

Mice were primed with 100 μg of p33-VLPs alone, injected subcutaneoulsy, or together with 100 μg of anti-CD40 antibodies, injected intravenously. Spleens were removed 10 days later and restimulated in vitro for 5 days with p33 pulsed splenocytes. Lytic activity of CTLs was tested in a ⁵¹Cr release assay essentially as described (Bachmann, M. F., “Evaluation of lymphocytic choriomeningitis virus-specific cytotoxic T cell responses,” in Immunology Methods Manual, Lefkowitz, I., ed., Academic Press Ltd, New York, N.Y. (1997) p. 1921) using peptide p33 (derived from the LCMV glycoprotein, aa33-42) labeled EL-4 cells as target cells. Briefly, EL-4 target cells were pulsed with peptide p33 (KAVYNFATM, aa33-42 derived from the LCMV glycoprotein) at a concentration of 10⁻⁷ M for 90 min at 37° C. in the presence of [⁵¹Cr]sodium chromate in IMDM supplemented with 10% FCS. Restimulated splenocytes were serially diluted and mixed with peptide-pulsed target cells. ⁵¹Cr release was determined after 5 h in a γ-counter.

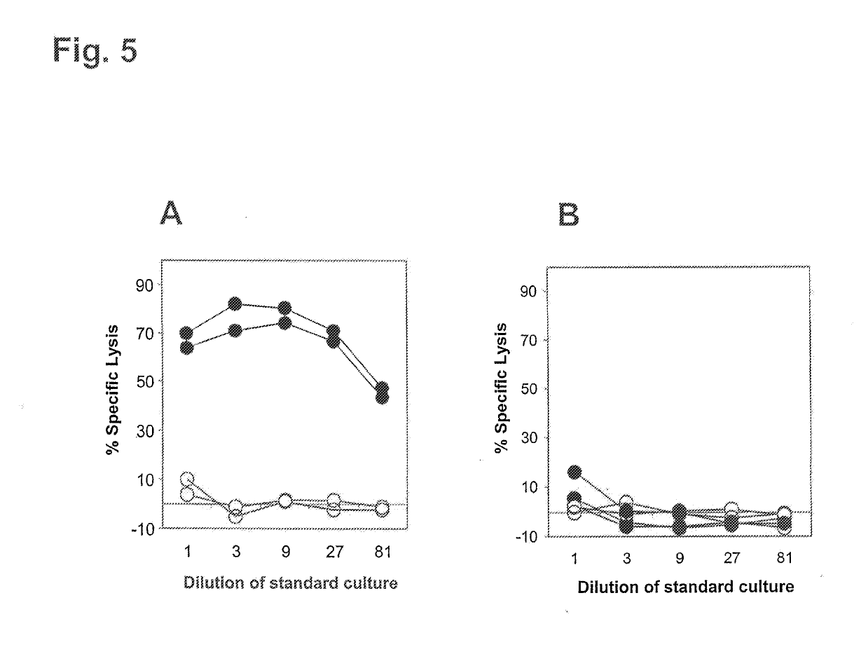

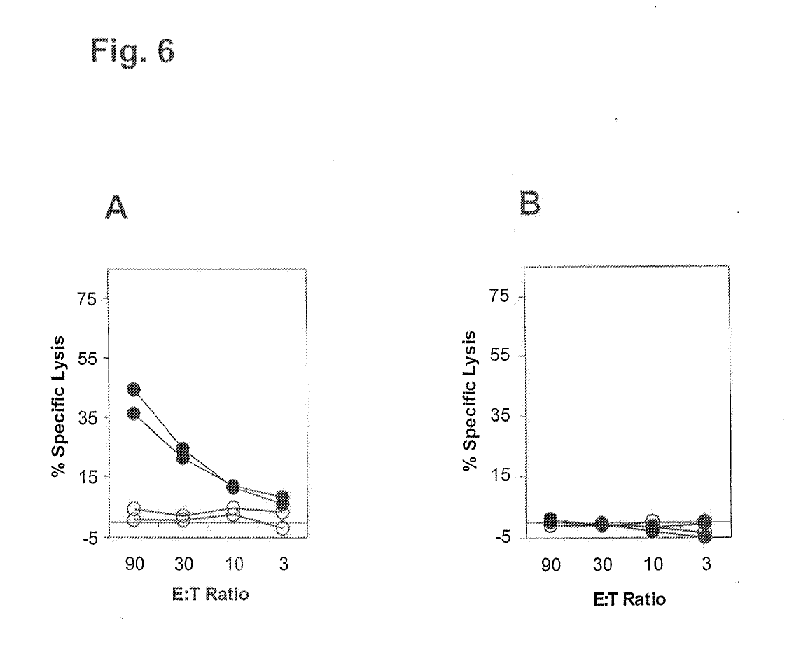

The results are shown in FIG. 5. Alternatively, splenocytes were removed after 9 days and tested directly in a ⁵¹Cr-release assay as described above (FIG. 6).

### Example 4

### P33-VLPs Injected with CpGs Induce Enhanced CTL Activity

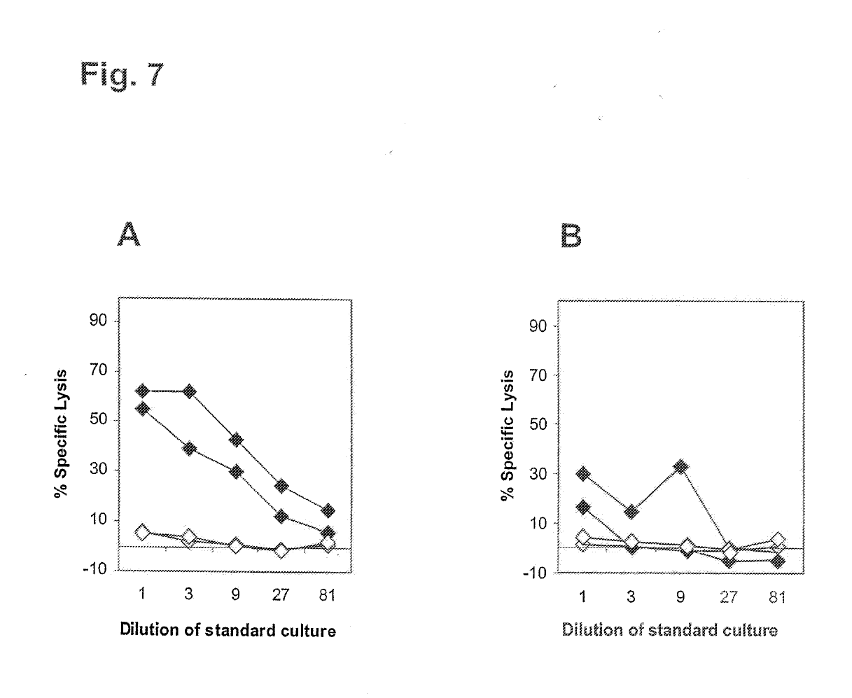

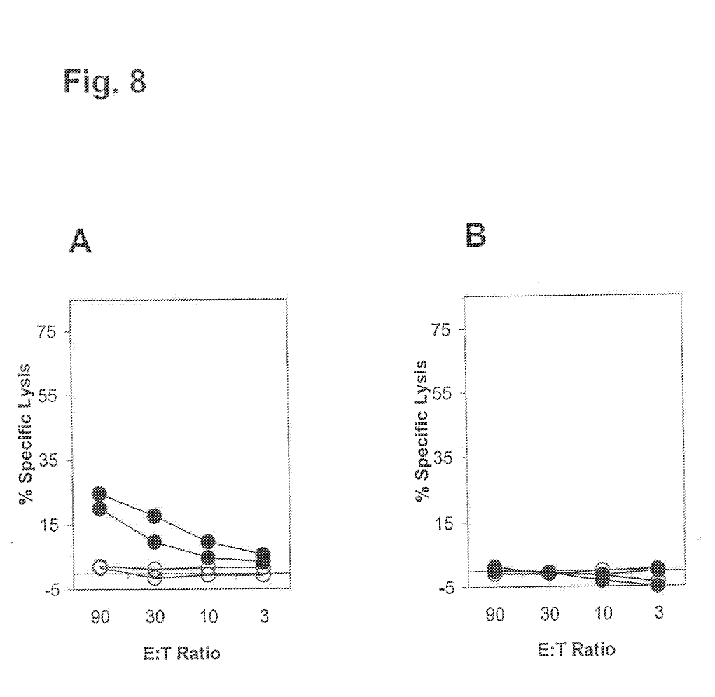

Mice were primed subcutaneously with 100 μg of p33-VLPs alone or together 20 nmol CpGs. Spleens were removed 10 days later and restimulated in vitro for 5 days in the presence of interleukin 2 with p33-pulsed splenocytes. Lytic activity of CTLs was tested in a ⁵¹Cr release assay as described above. The results are shown in FIG. 7. Alternatively, splenocytes were removed after 9 days and tested directly in a ⁵¹Cr-release assay as described above (FIG. 8).

### Example 5

### Anti-CD40 Antibodies are More Efficient at Enhancing CTL Responses Induced with p33-VLPs than CTL Responses Induced with Free p33

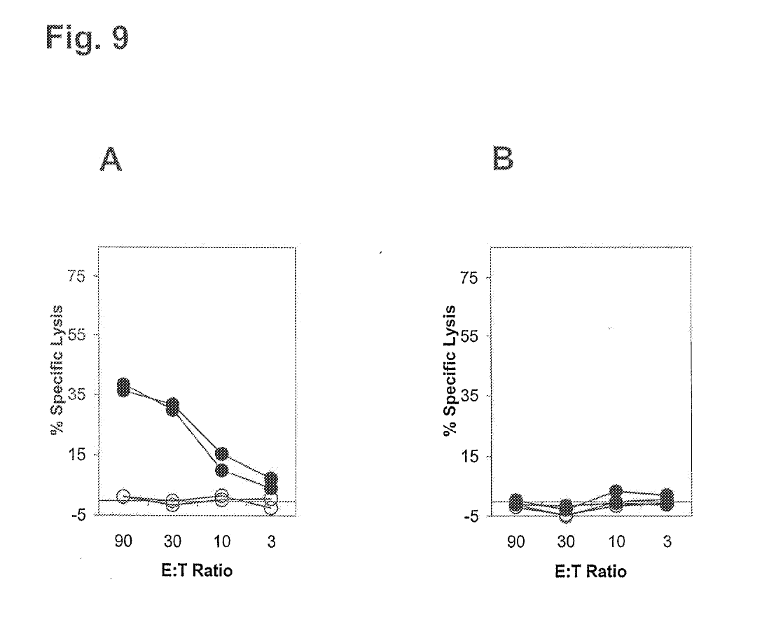

Mice were primed intravenously with 100 μg of p33-VLPs or the same amount of free peptide p33 together 100 μg of anti-CD40 antibodies. Spleens were removed 9 days later and tested in a 51Cr-release assay as described above. Results are shown in FIG. 9.

### Example 6

### P33-VLPs Injected with Anti-CD40 Antibodies Induce Enhanced Anti-Viral Protection

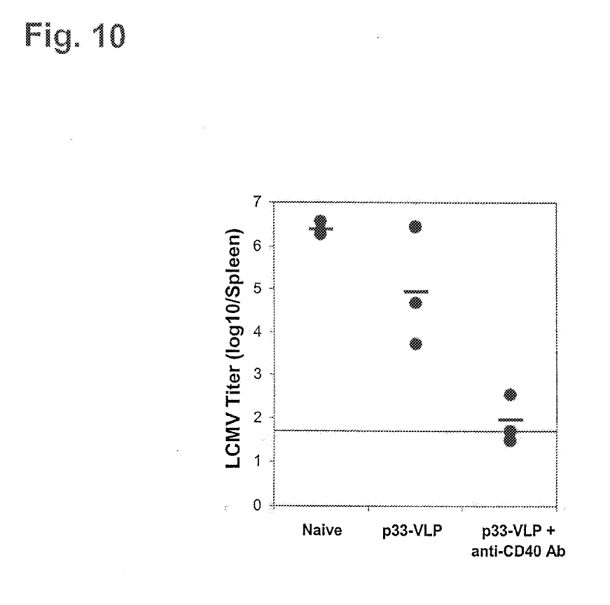

Mice were primed with 100 μg of p33-VLPs alone, injected subcutaneously, or together with 100 μg of anti-CD40 antibodies, injected intravenously. Twelve days later, mice were challenged with LCMV (200 pfu, intravenously) and viral titers were assessed in the spleen 4 days later as described (Bachmann, M. F., “Evaluation of lymphocytic choriomeningitis virus-specific cytotoxic T cell responses,” in Immunology Methods Manual, Lefkowitz, I., ed., Academic Press Ltd, New York, N.Y. (1997) p. 1921). The results are shown in FIG. 10.

### Example 7

### P33-VLPs Injected with CpG Induce Enhanced Anti-Viral Protection

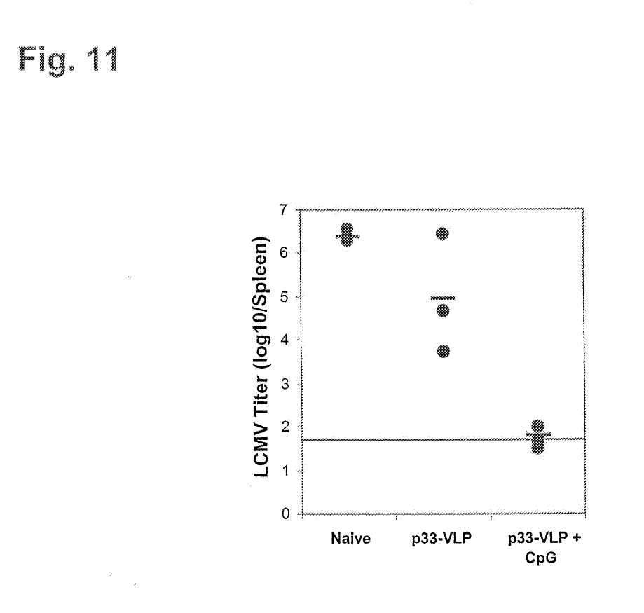

Mice were primed subcutaneously with 100 μg of p33-VLPs alone or together with 20 nmol CpGs. Twelve days later, mice were challenged with LCMV (200 pfu, intravenously) and viral titers were assessed in the spleen 4 days later as described (Bachmann, M. F., “Evaluation of lymphocytic choriomeningitis virus-specific cytotoxic T cell responses,” in Immunology Methods Manual, Lefkowitz, I., ed., Academic Press Ltd, New York, N.Y. (1997) p. 1921). The results are shown in FIG. 11.

### Example 8

### Anti-CD40 Antibodies and CpGs Induce Maturation of Dendritic Cells

Dendritic cells were isolated as described above and stimulated overnight with CpGs 2 nmol or anti-CD40 antibodies 10 μg as described above. Expression of costimulatory molecules (B7.1 and B7.2) was assessed by flow cytometry (Table 1).

### Example 9

### P33-VLPs Injected with Anti-CD40 Antibodies or with CpGs Induce Enhanced Anti-Viral Protection

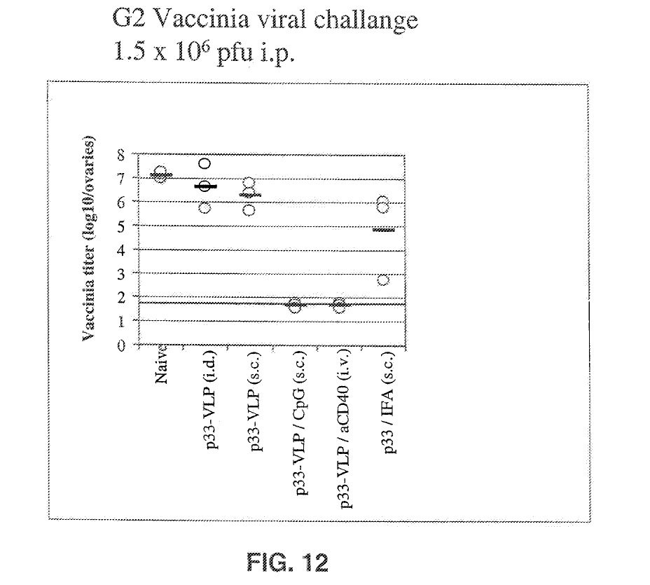

Mice were primed either subcutaneously or intradermally with 100 μg of p33-VLPs alone, or subcutaneously together with 20 nmol CpGs, or intravenously together with 100 μg of anti-CD40 antibodies. As a control, free peptide p33 (100 μg) was injected subcutaneously in IFA. Twelve days later, mice were challenged intraperitoneally with recombinant vaccinia virus expressing LCMV glycoprotein (1.5×10⁶ pfu), and viral titers were assessed in the ovaries 5 days later, as described in Bachmann, M. F., “Evaluation of lymphocytic choriomeningitis virus-specific cytotoxic T cell responses,” in Immunology Methods Manual, Lefkowitz, I., ed., Academic Press Ltd, New York, N.Y. (1997) p. 1921. The results are shown in FIG. 12.

### Example 11

### P33-VLPs can Boost Preexisting CTL Responses

Groups of mice are primed subcutaneously with 100 μg of p33 peptide in IFA or intravenously with 1.5×10⁶ pfu of recombinant vaccina virus expressing LCMV-GP. Twelve days later, half of the mice in each group are boosted subcutaneously with p33-VLPs (100 μg) mixed with CpG (20 nmol). Frequencies of p33-specific CD8⁺ T cells are assessed in the blood before and 5 days after boost by tetramer staining.

### Example 12

### CTL Responses Induced by p33-VLPs can be Boosted by Recombinant Viral Vectors

Mice were primed subcutaneously with p33-VLPs (100 μg) mixed with G10pt (20 nmol). Seven days later, mice were bled and subsequently boosted with recombinant vaccinia virus expressing LCMV-GP. Frequencies of p33-specific CD8⁺ T cells are assessed in the blood 5 days later by tetramer staining. Before boosting 1.4% of CD8⁺ T cells were p33-specific, while after boosting 4.9% were p33-specific CD8⁺ T cells.

### Example 12

### In-Vivo Virus Protection Assays

### Vaccinia Protection Assay

Groups of three female C57Bl/6 mice were immunized s.c. with 100 μg VLP-p33 alone, mixed with 20 nmol immunostimulatory nucleic acid or packaged with immunostimulatory nucleic acid. To assess antiviral immunity in peripheral tissues, mice were infected 7-9 days later, i.p., with 1.5×10⁶ pfu recombinant vaccinia virus expressing the LCMV-glycoprotein (inclusive of the p33 peptide). Five days later the ovaries were collected and viral titers determined Therefore, ovaries were ground with a homogenizer in Minimum Essential Medium (Gibco) containing 5% fetal bovine serum and supplemented with glutamine, Earls's salts and antibiotics (penicillin/streptomycin/amphotericin). The suspension was titrated in tenfold dilution steps onto BSC40 cells. After overnight incubation at 37° C., the adherent cell layer was stained with a solution consisting of 50% ethanol, 2% crystal violet and 150 mM NaCl for visualization of viral plaques.

Non-immunized naïve mice were used as control.

### LCMV Protection Assay

Groups of three female C57Bl/6 mice were immunized s.c. with 100 μg VLP-33 alone or mixed with adjuvant/20 nmol CpG oligonucleotide. To examine systemic antiviral immunity mice were infected i.p. 11-13 days later with 200 pfu LCMV-WE. Four days later spleens were isolated and viral titers determined. The spleens were ground with a homogenizer in Minimum Essential Medium (Gibco) containing 2% fetal bovine serum and supplemented with glutamine, earls's salts and antibiotics (penicillin/streptomycin/amphotericin). The suspension was titrated in tenfold dilution steps onto MC57 cells. After incubation for one hour the cells were overlayed with DMEM containing 5% Fetal bovine serum, 1% methyl cellulose, and antibiotics (penicillin/streptomycin/amphotericin). Following incubation for 2 days at 37° C. the cells were assessed for LCMV infection by the intracellular staining procedure (which stains the viral nucleoprotein): Cells were fixed with 4% Formaldehyde for 30 min followed by a 20 min lysing step with 1% Triton X-100. Incubation for 1 hour with 10% fetal bovine serum blocked unspecific binding. Cells were stained with a rat anti-LCMV-antibody (VL-4) for 1 hour. A peroxidase-conjugated goat anti-rat-IgG (Jackson ImmunoResearch Laboratories, Inc) was used as secondary antibody followed by a colour reaction with ODP substrate according to standard procedures.

### Example 13

### Staining of LCMV-p33 Specific CD8⁺ Lymphocytes

Groups of three female C57Bl/6 mice were immunized s.c. with 100 μg VLP-p33 alone or mixed with 20 nmol immunostimulatory nucleic acid. In alternative experiments, immunostimulatory nucleic acid was replaced by different adjuvants. 7-11 days later blood was taken and assessed by flow cytometry for the induction of p33 specific T-cells.

The blood was collected into FACS buffer (PBS, 2% FBS, 5 mM EDTA) and lymphocytes were isolated by density gradient centrifugation for 20 min at 1200 g and at 22° C. in Lympholyte-M solution (Cedarlane Laboratories Ltd., Hornby, Canada). After washing the lymphocytes were resuspended in FACS buffer and stained for 10 min at 4° C. with PE-labelled p33-H-2^{b} tetramer complexes and subsequently, for 30 min at 37° C., with anti-mouse CD8α-FITC antibody (Pharmingen, clone 53-6.7). Cells were analysed on a FACSCalibur using CellQuest software (BD Biosciences, Mountain View, Calif.).

### Example 14

### Immunostimulatory Nucleic Acids are Even Stronger Adjuvants for Induction of Viral Protection

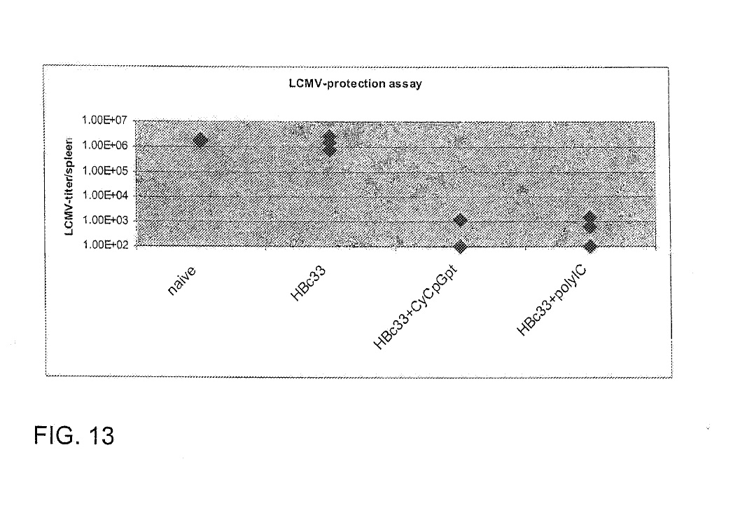

Mice were vaccinated with a HBcAg-fusion protein with the peptide p33 (HBc33) either alone or mixed with CyCpGpt or with poly (I:C). Viral titers after vaccinia injection were measured as described in Example 13. Oligonucleotide CyCpGpt lead to complete protection against viral challenge with LCMV, while poly (I:C) induced partial protection (FIG. 13).

### Example 15

### Different Immunostimulatory Nucleic Acids in the Presence of Antigen Fused to HBcAg-VLP Result in a Potent Antigen-Specific CTL Response and Virus Protection

The fusion protein of HBcAg with the peptide p33 (HBc33) was produced as described in EXAMPLE 1.

100 μg of HBc33 were mixed with 20 nmol of different immunostimulatory nucleic acids and injected into mice and vaccina titers in the ovaries after recombinant vaccinia challenge were detected as described in EXAMPLE 1. Double stranded CyCpGpt oligo was produced by annealing 0.5 mM of DNA oligo CyCpGpt and CyCpG-rev-pt in 15 mM Tris pH7.5 by a 10 min heating step at 80° C. and subsequent cooling to RT. Oligonucleotide hybridization was checked on a 20% TBE polyacrylamide gel (Novex).

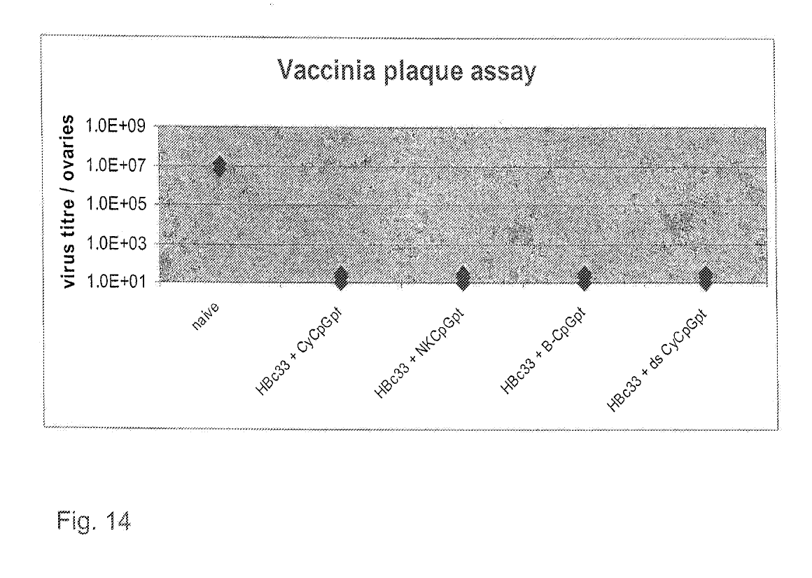

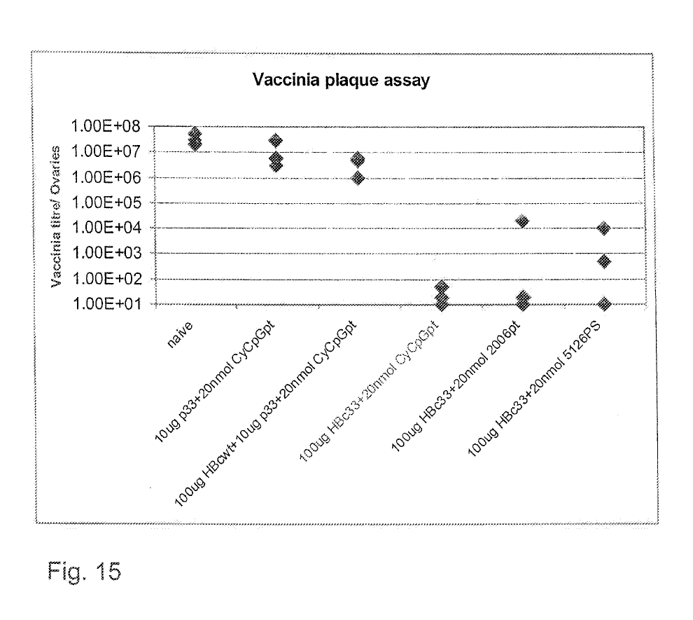

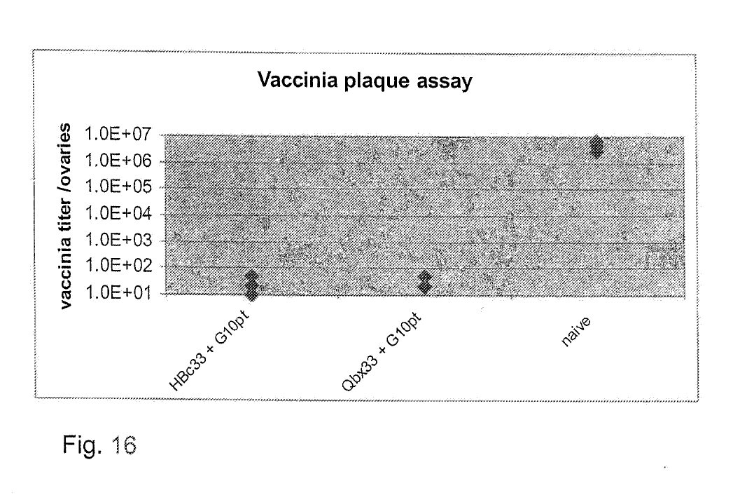

p33 fused to HBcAg in the presence of Cy-CpGpt, NK-CpGpt, B-CpGpt, dsCyCpGpt, 2006pt, 5126PS and G10pt did induce CTL responses capable of inhibition viral infection (FIG. 14, FIG. 15, FIG. 16). Both controls, peptide p33 mixed with CyCpGpt or HBcAg-wild type VLPs (HBcwt) mixed with peptide and CyCpGpt, did not induce protection. The fact that double stranded Cy-CpGpt also well as the immunostimulatory nucleic acid 5128pt that lacks unmethylated CpG dinucleotides, induced protection further confirms that a wide variety of immunostimulatory nucleic acids induce a strong CTL response against antigens bound to VLPs. The example also cleary confirms that coupling the antigen to VLPs is necessary to induce a strong CTL response. Furthermore, in a preferred embodiment of this invention, the unmethylated CpG-containing oligonucleotide is contains a palindromic sequence. A very preferred embodiment of such a palindromic CpG comprises or alternatively consists of the sequence G10pt.

### Example 16

### Antigen Coupled to the RNA Phage Q3 in the Presence of Immunostimulatory Nucleic Acid Results in a Potent Antigen-Specific CTL Response and Virus Protection

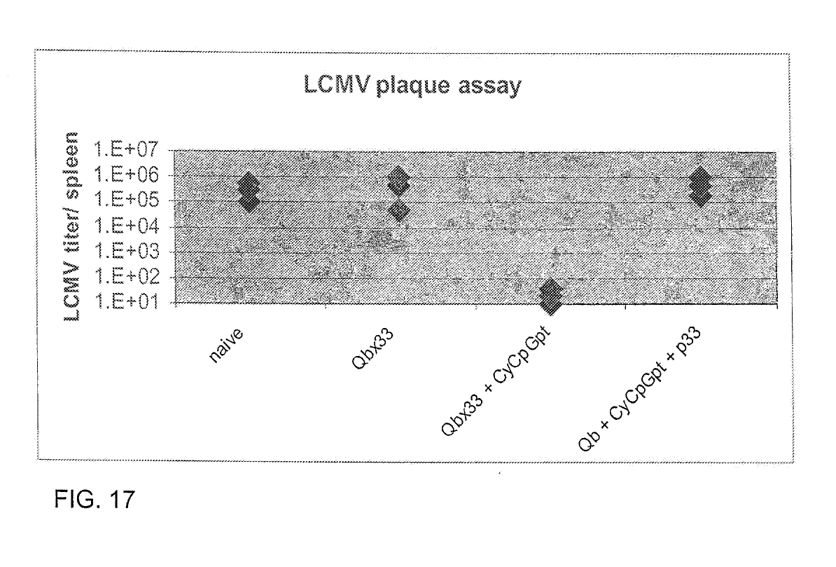

Recombinantly produced Qβ VLPs were used after coupling to p33 peptides containing an N-terminal CGG or and C-terminal GGC extension (CGG-KAVYNFATM and KAVYNFATM-GGC). Recombinantly produced Qβ VLPs were derivatized with a 10 molar excess of SMPH (Pierce) for 0.5 h at 25° C., followed by dialysis against 20 mM HEPES, 150 mM NaCl, pH 7.2 at 4° C. to remove unreacted SMPH. Peptides were added in a 5 fold molar excess and allowed to react for 2 h in a thermomixer at 25° C. in the presence of 30% acetonitrile. SDS-PAGE analysis demonstrated multiple coupling bands consisting of one, two or three peptides coupled to the Qβ monomer. The Qβ VLP coupled to peptides p33 was termed Qbx33. 100 μg of Qbx33 were mixed with 20 nmol CyCpGpt and injected into mice and LCMV titers in the spleen after LCMV challenge were detected as described in EXAMPLE 13. Controls included Qbx33 alone, or Qβ wild-type VLPs (Qb) mixed with peptide p33 and CyCpGpt. Qbx33 neither alone, nor mixed with p33 peptide and CyCpGpt did induce any protection against LCMV challenge. However, Qβ with coupled p33 in the presence of CyCpGpt did induce a CTL response capable of completely inhibition viral infection (FIG. 17).

### Example 17

### Different Immunostimulatory Nucleic Acids in the Presence of Antigen Coupled to the RNA Phage Qβ Result in a Potent Antigen-Specific CTL Response and Virus Protection

The peptide p33 with an N-terminal CGG sequence was coupled to RNA phage Qβ (Qbx33) using the crosslinker SMPH as described in EXAMPLE 16.

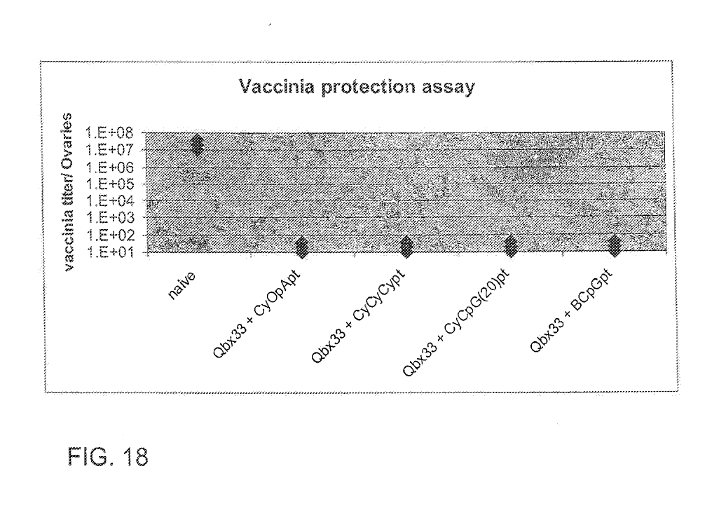

100 μg of Qbx33 were mixed with 20 nmol of different immunostimulatory nucleic acids and injected into mice and vaccina titers in the ovaries after recombinant vaccinia challenge were detected as described in EXAMPLE 13. Qβ with coupled p33 in the presence of CyOpApt, CyCyCypt, CyCpG(20)pt, BCpGpt and G10pt did induce CTL responses capable of completely inhibition viral infection (FIG. 16, FIG. 17, FIG. 18).

### Example 18

### Antigen Coupled to the RNA Phage AP205 in the Presence of Immunostimulatory Nucleic Acid Results in a Potent Antigen-Specific CTL Response and Virus Protection

AP205 VLPs were dialysed against 20 mM Hepes, 150 mM NaCl, pH 7.4 and were reacted at a concentration of 1.4 mg/ml with a 5-fold excess of the crosslinker SMPH diluted from a 50 mM stock in DMSO for 30 minutes at 15° C. The obtained so-called derivatized AP205 VLP was dialyzed 2×2 hours against at least a 1000-fold volume of 20 mM Hepes, 150 mM NaCl, pH 7.4 buffer. The derivatized AP205 was reacted at a concentration of 1 mg/ml with either a 2.5-fold, or with a 5-fold excess of peptide, diluted from a 20 mM stock in DMSO, for 2 hours at 15° C. SDS-PAGE analysis confirmed the presence of additional bands comprising AP205 VLPs covalently coupled to one or more peptides p33. The coupled AP205 VLPs were termed AP205×33.

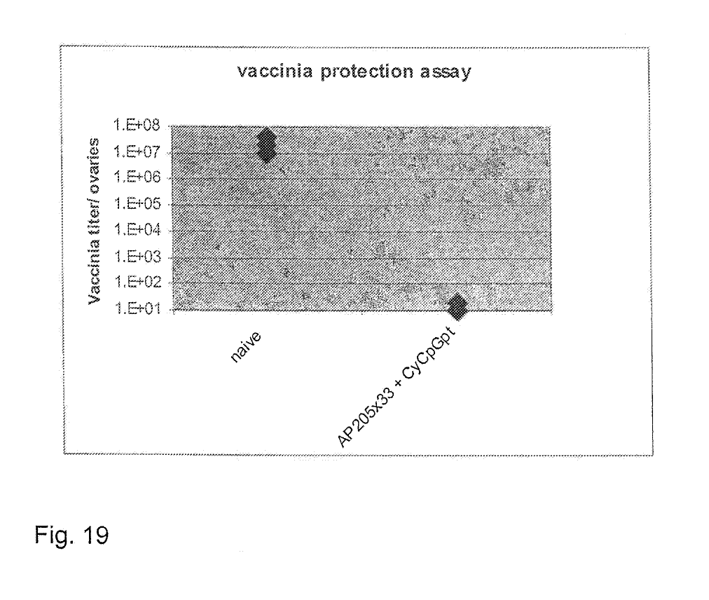

100 μg of AP205×33 were mixed with 20 nmol CyCpGpt and injected into mice and LCMV titers in the spleen after LCMV challenge were detected as described in EXAMPLE 13. AP205×33 mixed CyCpGpt did induce complete protection against vaccinia challenge (FIG. 19).

TABLE 1
In vitro maturation of DCs stimulation with anti-CD40 or
CpGs

| treatment | B7.2* | B7.1* |
|---|---|---|
| Medium | 302 | 79 |
| Anti-CD40 | 530 | 241 |
| CpG | 520 | 238 |

*Mean fluorescence intensity

1. A composition for enhancing an immune response against an antigen in an animal comprising:
(a) a virus-like particle bound to at least one antigen capable of inducing an immune response against said antigen in said animal,
wherein said virus-like particle comprises at least one first attachment site comprising an amino group of a lysine residue;
wherein said antigen comprises at least one second attachment site comprising a sulfhydryl group of a cysteine residue;
wherein said virus-like particle is a virus-like particle of RNA bacteriophage Qβ comprising coat proteins having the amino acid sequence of SEQ ID NO:10, and wherein said coat proteins of said RNA bacteriophage Qβ comprise said lysine residue; and
wherein said first attachment site is associated through at least one covalent non-peptide bond to said second attachment site to form an ordered and repetitive antigen array; and
(b) at least one substance that activates antigen presenting cells in an amount sufficient to enhance the immune response of said animal to said antigen, wherein said substance (b) activates toll-like receptors in antigen presenting cells.

2. (canceled)

3. The composition of claim 1, wherein said virus-like particle (a) is a recombinant virus-like particle.

4. (canceled)

5. (canceled)

6. The composition of claim 1, wherein said antigen (a) is a recombinant antigen.

7. The composition of claim 1, wherein said antigen (a) is bound to said virus-like particle by way of a linking sequence.

8-21. (canceled)

22. The composition of claim 1, wherein said antigen is a tumor antigen, and wherein said tumor antigen is selected from the group consisting of:
(a) Her2;
(b) GD2;
(c) EGF-R;
(d) CEA;
(e) CD52;
(f) CD21;
(g) human melanoma protein gp100;
(h) human melanoma protein melan-A/MART-1;
(i) tyrosinase;
(j) NA17-A nt protein;
(k) MAGE-3 protein;
(l) p53 protein;
(m) HPV16 E7 protein; and
(n) antigenic fragments of any of tumor antigens (a) to (m).

23-29. (canceled)

30. The composition of claim 1, wherein said toll-like receptor activating substance is selected from the group consisting of, or alternatively consists essentially of:
(a) immunostimulatory nucleic acids;
(b) peptidoglycans;
(c) lipopolysaccharides;
(d) lipoteichonic acids;
(e) imidazoquinoline compounds;
(f) flagellines;
(g) lipoproteins;
(h) immunostimulatory organic molecules;
(i) unmethylated CpG-containing oligonucleotides; and
(j) any mixtures of at least one substance of (a), (b), (c), (d), (e), (f), (g), (h) and/or (i).

31. The composition of claim 30, wherein said immunostimulatory nucleic acid is selected from the group consisting of, or alternatively consists essentially of:
(a) ribonucleic acids;
(b) deoxyribonucleic acids;
(c) chimeric nucleic acids; and
(d) any mixtures of at least one nucleic acid of (a), (b) and/or (c).

32. The composition of claim 31, wherein said ribonucleic acid is poly-(I:C) or a derivative thereof.

33. (canceled)

34. The composition of claim 1, wherein said immunostimulatory substance is an unmethylated CpG-containing oligonucleotide.

35. (canceled)

36. The composition of claim 34, wherein said unmethylated CpG-containing oligonucleotide comprises the sequence:
5′ X₁X₂CGX₃X₄ 3′
wherein X₁, X₂, X₃, and X₄ are any nucleotide, and wherein at least one of said nucleotides X₁, X₂, X₃ and X₄ has a phosphate backbone modification.

37-40. (canceled)

41. The composition of claim 34, wherein said unmethylated CpG-containing oligonucleotide comprises a sequence selected from the group consisting of:
(a)
TCCATGACGTTCCTGAATAAT;
(b)
TCCATGACGTTCCTGACGTT;
(c)
GGGGTCAACGTTGAGGGGG;
(d)
ATTATTCAGGAACGTCATGGA;
(e)
GGGGGGGGGGGACGATCGTCGGGGGGGGGG;
(f)
TCCATGACGTTCCTGAATAATAAATGCATGTCAAA GACAGCAT;
(g)
TCCATGACGTTCCTGAATAATTCCATGACGTT
CCTGAATAATTCCATGACGTTCCTGAATAAT;
(h)
TCCATGACGT TCCTGAATAA TCGCGCGCGC GCGCGCGCGC
GCGCGCGCGC GCGCGCGCGC G;
and
(i)
TCGTCGTTTTGTCGTTTTGTCGT.

42. (canceled)

43. The composition of claim 1, wherein said unmethylated CpG-containing oligonucleotide is palindromic.

44-77. (canceled)

78. A method of enhancing an immune response against an antigen in an animal comprising introducing into said animal the composition of claim 1.

79-194. (canceled)

195. The composition of claim 1, wherein said unmethylated CpG-containing oligonucleotide consists of the sequence GGGGGGGGGG GACGATCGTC GGGGGGGGGG.

196. The composition of claim 1, wherein said first attachment site is a side-chain amino group of lysine residues of said VLP or of at least one VLP subunit.

197. The composition of claim 1, wherein said antigen is a microbial antigen.

198. The composition of claim 1, wherein said antigen is selected from infectious virus, infectious bacteria, parasites, or infectious fungi.

199. The composition of claim 1, wherein said antigen is a recombinant antigen.

200. The composition of claim 1, wherein said antigen is selected from the group consisting of:
(a) a recombinant polypeptide of HIV;
(b) a recombinant polypeptide of Influenza virus
(c) a recombinant polypeptide of Hepatitis C virus;
(d) a recombinant polypeptide of Hepatitis B virus;
(e) a recombinant polypeptide of Toxoplasma;
(f) a recombinant polypeptide of Plasmodium falciparum;
(g) a recombinant polypeptide of Plasmodium vivax;
(h) a recombinant polypeptide of Plasmodium ovale;
(i) a recombinant polypeptide of Plasmodium malariae;
(j) a recombinant polypeptide of breast cancer cells;
(k) a recombinant polypeptide of kidney cancer cells;
(l) a recombinant polypeptide of prostate cancer cells;
(m) a recombinant polypeptide of skin cancer cells;
n) a recombinant polypeptide of brain cancer cells; and
(o) a recombinant polypeptide of leukemia cells.

201. The composition of claim 1, wherein said antigen is a recombinant polypeptide of Hepatitis B virus.
